SEQUENCE LISTING (4 sequences)

SEQ ID NO: 1 - DNA, 36 bp, Artificial Sequence
  Description of Artificial Sequence Synthetic oligonucleotide
aatatttcgcttattcatgtagatagaattatttaa

SEQ ID NO: 2 - DNA, 22 bp, Artificial Sequence
  Description of Artificial Sequence Synthetic primer
cctttccacctttgctgtagga

SEQ ID NO: 3 - DNA, 18 bp, Artificial Sequence
  Description of Artificial Sequence Synthetic primer
gtccaggccggaacaaca

SEQ ID NO: 4 - DNA, 180365 bp, African swine fever virus
atttttttgcgcgccggaataattattttttttgcgcggaaatatttttgcagtggcgattgatttttttttcagttgtctgcccgtggcggaataattattttttcgccggaaagcattttgtagtttcacaagatgcagaatttggcgggccggaataattattttttcgccggaaagtattttgtgtaccgtagtttcactagatttcgcggtgcataccggtgggcgataggtattaaaataatttgtactgctgtaggcgttaaagattaaaattattattactgctgtaggcgttaaacattaaacttattactactgctataggcgttaaacattaaaattattactactgttgtaggcgttaaacattaaaattattactactgctgtaggcgtcaaagattaaaattattactactgctgtaggcgttaaacattaaacttattactactgctataggcgttaaacattaaaattattactactgttgtaggcgtcaaagattaaaattattactactgctgtaggcgttaaacattaaaattattactactgctgtaggcgttaaacattaaaattattactactgttgtaggcgttaaacattaaaattattactactgttgtaggcgtcaaagattaaaattattactactgctgtaggcgttaaacattaaaattattactactgctgtaggcgttaaacattaaaattattactactgttgtaggcgttaaacattaaaattattactactgctgtaggcgtcaaagattaaaattattactactgctgtaggcgttaaacattaaacttattactactgctataggcgttaaacattaaaattattactactgttgtaggcgtcaaagattaaaattattactactgctgtaggcgttaaacattaaaattattactactgctgtaggcgttaaacattattactgctgtaggcgtagctaaattttaatgttttttcgtaaaaaatcgccagtcacaaaaaaaagcagccatgtatatctgtgatgtttggtgttttatctacatgctattggcattcctgatattcatgttaaagtgctatactataaccatcttatgtgggctctttatacatcttattggcaggaggcccactatatatatcctgttaagtagtttagctgtatactatttaatgtttttcgcttgatcaagaaatatacaaaaatctaagtgcaccattttatgggcatcaatcaatcacagcattctatgggcgtgaatcaatcacaccattttatgggcgtcaatcaatcacaccattttatgggcatcaatcaatcacagcattctatgggcgtgaatcaacacattattggggacagctacctgatatcgtgtatggtggtggcaccgtgttgtttgaaggcgtgtatggtattagcgccatgttgtttgaaggcagcgcgttagtaacaacgttttttttcgtatgacaacagatatgtataagatagcaaattaatgctattgtcatcaatgagaaacataagtagatatagacctgcctcttggtagtccaatgatccatgttgcacctaaaaatcccaaaaaaaaacattaagttttggagggtaagattggtttttcaccattggtaaagattattattctaaatgtttaccccatagatgtgaaacaatgattcttcatatattaacatattttttgacttatacttttcttcatctagtaaggcgttaattttttccggatctgtcgtttttattgataaaagagaagagtctggactgtaatttttaaataataagatatttattaatatccaattattcgtttgactcgctatttccatgctctcttcaaacgcatcagctcctaaatctatacaaaggaataagttaccttcacaaaaattcattaccgaggtaatcattgcccgattaatgtcagctcccaacatacaacaataatatatagttgtataattacaatcatacatacaggccaactgcatcatttcatcaatgtctatattcgtcttctctttgttataaatttcatgaaggtcaaagacgttgttataagcaaccccacatattaaccgccaatctttaaaatgactatatcgttgataaaaatattggatggcttcagtaagcttatatagtatcgccatactataccaatacctagttagcatttcgttgaatgaaatattatccaatgtaaagttgattgataatgtatctagttcaccaaaaattcttaatttcagttgagcattatttaggaataggggattatcagataataattcatggcatagaataatattactgctagttttaacatactgtacattataaaatatttctaaaattttattttcactcaaagctttcctcgcacctaacttttggcataggtcctgggtgcactccatattgacagtaaccaacccaaagctgatgtctgcaccccattcggtaaacagctctattaaaccatgattgttttcctgtacagccttcattaatgcaacatttaatgttaaaccatgtttaaaacttgctgtttttattaatatttgtttatctatacaagtatgatgaatcgtaattggggcttcatgccaccacaaaccacaacgctctaaaatacaataatcatctttgaacacaggctgtgtagctagtacttttttagtaagtgcttgtaaagtagatggcatcttctatctgcaaaataattatttccgaaaaaaaaatcaaattaaaatactaaattctatttttttttaataaagcctgtaaattatataataaatctcgcccaccgtattattttcggacacaactttttatacctcattatatttttagatctatagttttttaacaaggcattaattttttctggatctgtcgtttttaaagataaaagagagacgtttgaattataaaaatctttaaatgataatatttctactaatatatcatgattcttttgttttgctaattctaagctctcttcgaaagcattagctcctaaatctatacaaaagaacaagttattagtataaaagttttttaccgaagtaaccattgcccgattgatgttagcccctaatacaaaacaatagaaaatggttaaaaaattgctatctctcatacaggccagatacatcatttcatcaatattcatatcaaccttttttatatgatatatttcatgaagatcagacacgttattaaaagaaagcccacatattagccgccaatctttaaaatgcctataacgttgataaaaatattggatagcttcagttaggtgatatcgtaccgccatactataccataacctagctagcatttcgttcaatgttatttcattcaatataaagttgatcgatatcttctctagaaaacaacaaattattatttttaattcctctatattctggaaaaggggattattagataacaatttatggcatagaataatattactactagttttaatacgatgtattttataaaatatttgtacaatatccatttcattcaaaatttttgcgcctaactcccggcagaaattccaagtatgctccgtattgacagtgactaagctagagttgatgtctgcaccccattcggtaaacaactctattagatcatagttgttttcctgcacagctttcattaatgcgagatttaactctaaaccatctttaaaaattgctgattttatcatcaattgattatcctcattagtagaaaacataattggagctccatgccaccacaaaccacaatatttcaaaataaagtagtgatttttggatatgtgctgtgtggccagtatttttttagcaagagcctgcagagaaagtggagtagacatttttttttgcaaaatggtttaagttttaatgttttcaagaatacagattggataaattaggttgacattagtaacagaaagtattcaatattatgtagacctataaaaaatgagaccctccaaaaaataataaacaacacaaaaaaaatatgtttaatattaaaatgacaatttctacattgcttattgctcttattatactagttattattattttagtcgtatttttatattataaaaaacaacaaccaccaaaaaaggtctgtaaagtagataaagattgtggtagtggagagcattgtgttcgtggaacatgcagcacattgagctgcttagacgctgtaaaaatggacaaacgaaatattaagatagattctaagatttcctcatgtgaattcactcccaatttttaccgttttacggatactgctgccgatgagcagcaagaatttggaaaaacacggcatcctataaaaataactccatctccaagtgaatcccatagcccccaagaggtgtgtgaaaaatattgttcatggggaaccgatgactgtacaggttgggaatatgttggtgatgaaaaggagggaacatgttatgtatataataatccacatcacccggttcttaaatatggtaaggatcacatcatagccttacctagaaatcataaacatgcataaataaatacattaggctcatcgtatcctttttaaaatccataaatattcgtttgatatatgctgaaatttttataaaaaaaaataactatttcttataaatcatctagaaatagtcctcgttttgatcggtttatatcttataatattgtgcatcgatgcacaactgctttttttggtccttctggaacatcattatattttctttcattaatataccattcagatgtaaatgttgaataatttttatggcaacaatctaccactgaattatatttagtaacatctaatacatcgcttgttttatcaggctcagctttataatcttgataatttttgttatcagcttctaaagctccatcattatttttcaaagaagtgtccataatcatgtttggtaaaaacactttaagttttaatatgatatttaaaatggttgttatataaatttaccgcttacaggtaatctttattcagtatcataaactatacttttgatggttcagtattctgtaaatcggtattttgtgattcagtattctgtaaatcggtattttgtgaatcagtacatttattatcattaatatttttaggctgattttccaatgttttattgttgcaatgagcctgctcctcctttaacggggaagtgtctgttggagtcatctgtttaggaagagtatcatccatatctattatgaagaaaatatataaatattgatatacaatcaaaaatatttttgatcacgtctttgttatctatcgatattgttgataacgtcttgaataacctacatcatttttttacataaaaaaatagatataatttttattatatctcaattatcttaagataattatcaaagtaactattttaaagataattatcaatacaacaaatatcataagctaacatatttttcgaataatagttttttagtaaagtattaatcttttcaggattagtttcttttgataataagataggattcgctttataaatttttaaagataatatattcacaatgatagaataaccgtatatatctgctaatgtcttactgtgttcaataacattagcccctaaatccatacaaaagaacatattttcaatacaaaagttttttaccgagattaacattgctcgattagcgttggctcccaatgcaaaacagtagtaaatggtcaaaaaattattgtcgcgcatacaggccagctccatcattttattaatactcatatgaattttcgttgtgttacatatttcatgaaggtcaaacacattgttgaaagaaagtgcacaaattaatcgccattcatcaaaatgcctgtattcttgacagaaatattgaatagcttctttaagattatattttaccgctatgccataccaatatttggttagcatctcactaaatgagatctcatttaacatagaatttgttggtaaatccttcaactcccaataaataatcatccttaaatccaccatgtttacattttgtaaaaaagggttattagaaaataattcatgacacaaaatgacattactacttgttattttacactttgtttcaaagaaaaatcgtaaaatttcacttgtctcaagctcttctttagcccccaattttcggcataggtttcgagtatgctcgttattaataaaaagtaacccataattaatatttgcgccccattcagtaaacaacatgattagatcttcattgttttccttaactgtcaataccaatgcagtattaagctttataccctctttaaagcataatgtccttatcattatttgattatcatcatctatataaattgagataggagcttcatgccaccataaaccacaacgctctaaaatataataatcatctttagatacgtgttgcgtggccaatgcccttttagcaagtgcttgtaaagtcgatggctgcatgtttattttgttaaaaaaaaatcaaattatcgggtaaacataaggatcaacccgtagttaatatttgcagtgacatttttaacaatgaattatgataaaaaaataattcatgactatctatcatggaacccatctttaacattaaagaaaaaccagatcatcatttttttttgttgtattaggacttcttcaaattattacccacattttatctaaaaaaataaaactacataatatcttgtttcttcatcaaattatcataccatttaaagtgtgtaggttgggaacattccataatatgggtattaaagtgtttatatattttttcataggaacatttatttggcagatgtgttgtccaacaatcatatctaataaaatcattttcacctatggggggatgattcttaaaagccttattcttacagatgccattttgacagtcccagcaaaagttacaatattttccatgagtacaccaatgttcaagctctcttttgggaggaatgctgccaattttatgttttttatcttctaactctccatacagcatcagttgggaaagcagaaaggagattaccaggaagaccagcaaatacataacagtctgtaaactacgtttgcgaatgtaattcacagctaaaatacatcccgcaagataaaatccataggttaacaatttctgccattttcgtatatcagcctcatgcctttcatggtttatgtgttgtggacattctgttcggtacgttttatgaggctttatagaagttatgtagtagatacagaaatcattgcgacgataaatactgcagttagctatatagtcattttcaagaatgggagaatggtttttaaaagccttgttcctacagataccatcttgacagtcccaacagaatctacaatggtttgcataagtgcaccagtatccaagctccttttcaggtggggtacttgttcgttccaggggctctatctcatatgtataaagaagagttggaatagatagtagggtgaatatttgcagaccaagcatggctacttgtgaacaagtggctgctcgtcagcaaatagctgctcgtcagcaaatggctgctcgtcagcaaatgctgcttgtgggtaagccaataaatgggccatacccttgaaaggagaattcagtttgataaaaaaaataacgagttttctaataacccggtcaagcatttattaaatgagtggcatcagatgtctgcatcgtgcattctacttggaaaatgggcccatctctaatatatttatactgacggtgaatcatacagtgttccatgggatagctatgctcctgtacagaagacatatctttaagaactttattcttacaaagaccatcttgacaagtccagcaaaaccgacaatttttcacatattgacaccagtatcttagctcctcttccaggagattgtcggtcgaaaaaccctgtagactagttagaccagctagcagcaagccgaggtaactaaagaacctcattgtagtgttatattacgaaaaaatatgttaaaatttggaaaaaaaagccctttttatagatctggaaaaaaattttcacaaatctaattaaaatcctcacagatcttccttttcataaattttccttcccaatcggtgtggacgattgtgaggtactggattagaacaatccataacatggtaatgcccatttccttcactatatgtgcactggttataccagcgagaaacctcacaagatgtcaaataactgttctcaataatcaatggcatgctctcattcaccttgttcttgcaaattccatgtgcacattcccagcaaaacttgcagctttccatgtaagtacaccagtatccaagttcttcttgtggaggattatccgttgaacgaagatgccctcctgcctgagtaggtagtcctaagacctgattggccagcaggccaagaattcccaagaagatcaccaacattgctacggctggctgaacagctgaccgaatagctagccaattagcaaaccaagtgactcgcccctctactcttaatatgaaaatttaagattcgattcagcatttttcctgtgttttacagagaaaagatatttttagtctatgaatgcacatggttccgcacattaaaaaaataaattatttatttagccagcgggaaactatacatggatgcacatactcattttcaagaattgttgtatttcccaacttattcacacaaaggccattttgacagtcccagcaaaactggcaacattccatgtaagtgcaccaatattcaagttcattctctgaaggattttccgttggatgaagttgtccaacgagctggcttgagacctggttggccagaaggccaagaattcccaagaagattactaacattgctacggctggctgaacagctgctagatagctagctaattagcaaaccaagtgacgccctctctattctttatatgatgatttaagattgagtccaacatttttccaacgttttacaggaaaaaggtatttttaactatggaattaatcttaacccactatttagaaaaaaataaaagacacatttatttaattttagctgaggcaaatttgctgacaaactctagagttcatcttttttccattccttcctgaagtatttgtaaggattccaagaccatggctgagaacaattcatggtatggtagatacctttatctaaatcatacatacatttatccagccaacgggacaccagacatgctttcacatactcattcttataaatggccgacccatctattttgtttatacaagtgccatcttggcagtcccagcaaaattgacaactttccatgtaggcgcaccagtattcgagttcttcctctggaggatcctctgttggtcgaagttgtccaacgagttgacttgaaacctggctggccagaaggccaagaattcccaaaaagatcaccaacattgctacggctggctgaacagctgactgaatagctagccaattagcaatccactgtacttttcataagatcatttaagattcggtcgacattttttcaatagtttgctaggaaaaaatttttaatcttatagattcacaccacttcattctcatgcttaggaaaaaataaaccaaatcttacaatgtatctggatctaatgagaaactagaattcatcttttttcaactcctttctggaatgtttatcctttttccactccttccttgcaattttataaggattccagggctttgggtcagaacagttcatgctatgcatcatattctatttaacaatctcctaaataaatgaacatctttttcattttaacatgatcaccaatagtcattttgcgaaaaggcatacatatgatcaatatcagacctacaatgaatgtttccataatatccctttatcgcaataattctatttttgcattccgatatctcatcatctgtgctattatatgtttccataactgtttcatcatcaaacataaatcctattaaataagcaaagcgctttaatcccggatagattttcaccatttttctaagagccgtgtatagcttgtaataaatggccagaaatatgcaataaagcgtagaaagagagtaatttttggcataaaaggttttgaaggtttgatgaatggctaaatcgcagataatataagatacgatattaaagcgcacctgttcacgcagatttgttgaaaacttcgtagaaagattcaacaaataaaaggttattaatagttgctcatcattctccttatacgacatcgtcagacgctctaagattttactactggacacatctgccacatgttgaacatttaaagcctgttcttcttctgtgttacgacaaaagagccgcgcatattcaggtgaagctccccaggataacaacgtccgtgtcacggctaaattttttttgacgatgactttcatcagaaataagtctttatttttgcattgatcactatgcgaatttgtatagttgacgccgttgcattgagtacattgatataatgttttgcaattccagcgtagccctaaatggtataaaaggactgtattttcgacgcaagcatgctgattaacgatgtttttgagacaacacgttgttaaggacaccatattgtctccaatttgttggataaaagtctttactaaaaaaataggtttttagttttaacaatcgagattttattatttggatatagcatcatcaaaaagatttatgttaaaatttaatttgtcttacaaaaagtacagattcaataattggtttaactatttagtttaactatttagttaaattacttggttagcaatgtttgagctttcttccattcttattcgaggggggggggggggggggtcctaatagttttgatacttttgttgtggattgttgatgaaaactgtacagatgctaaagcaatggcatataatattaattgtactgtttaatgtgagtttataagtatgtatcaaaatttatttttttttataaaagtacagaaattaatgttagcttaaactttagtttagctatttgagttaatgatgtttaacctatcttccatgcttatcagggggggggggtcctaattgttttacgttataaatttgatcaaaatttaatttcttataaaaaaggtacacatcaacattgctcatttaagtttcataatgtttgattcattacttcctacaattactggggggggggggtctttaatagcattaatattattatggtttgctgactatttggaatttatagaagcaaaaatagatacttttactaatagcagtgcattataagtgatcaaaatttaatatctcataaaaaaggtacacatcaacatcattcatttgtttcatgatgtttgattcattactttctacaatcactgggggggggggggggtctctcatagctttagtattgctatggtttactaattattatgtagaatttataggagcagatatatatggtggtaattgtactctataagtttataaaaatttcagtatattttttttgttaatataacagaaatgatttaaattacaaaaaaaagagaatcatcatgctattatacattgttattatagtagcatatgtaagttataaattggttccaaaacaatattggccaatactcatgtttatggcttacatggtatatacccatgaaaaattagatataaatgaaagatccggattttggaaatatattatagcaaaactattcagatgccacggatgtgaaatatgtaagtgatattaaaaacataatacgaagtgtaataattgactattttttgttgaatggaaactgtttttgagtaatatgttgaacataatggtttttatagatgttaaatcatttgattgtagtaattgtactaaatgatgtgagatataaacctagttcaatcaaaatttaatttttttataaaaagctacagagtgttttattaaacgtagcttatttaaaaagttacacaatgttaaaatctctacttactttaattctttgtggggttttattaactttatccatattatggcttattacttaccatgtagaacttatagaagcaatagatgatttctacgactgaaatatagaatagttcattttctatttgtaaaataatgatttatattctctcctaaaaatacgatcgatcctttatatgctttgaaaacgaatattaacaacttgatttttttttctataaataaactataaatgaaaatagtaaaactcatagagtcttataagtgaacatccatcatgttactcaaacgttggactattaaaagatactccgtgtgcattattgcttttaatcagtatgattactttatacgaagccgctattaaaacgcttatcacacaccgaaaacagattttaaaacaccccgatagccgtgaaattttactagctttggggttgtactgggataaaactcatattcttgttaaatgtcgtgaatgtgggaaaatgagtcttaccggaaaacacagtacaaaatgtattaacattaattgtctacttattcttgccataaaaaaaaagaataagcgtatggttgataccttgataagaatgggtgcggatgtaacatatatacatcttttaaagaataagataaaactgtcatacaaccagctgtctatgcttaaaagcaactcgcagatttcgttgaaggagcttcatgctatatgctaccttttatatggtcggcttcccaaaaaaattaaacaagggatgcaactgtgtaaaacaatggcgggactatgtggtgaacttttatgtgcatttttagctccgtaaatgtaatatatgtatataaaacaaatagataataccaaaatatattctatgtacataacatccggaaaattatttttttttctcatacccttaaatataaaaatattgggtttcttcactaaactttagaggtaaaaatttttctttgttttgcatcatcatgtatgggtttaggttatcccagggattgtttatttgaatatttcctaaataggaacacaacgccatgatcatatatctttcattctggtaagctttttgatacatcttcaaagatgccgtacctccgagtgtgtaacagcaaacaaacgtccgtacttttccatgggtcgcagcccattccattccgtagctcagcatcctttgctgtatttttttattcgctttataaaaaaagtatttcatccattccacgttctcataaaaacaggcacttaaaaagagcaccaggtttagtgtagtcttattatagaatgtaggaatgtatgttttagttatttttttcaacgcctgtttcataccatgttttaccgccataaaaatacaaaaccaataccaactttttctataaaaggttttgctgtacacatgtaaacgagcaaaatatatttcaaactctaaattctttttataaaaaaactcgagacagtcatttatgttacgacatcttctaaaaacctcaaaaacagtaattaattcactgtcgctgtagaaatgttcataagctaactgtttaatgtctttaggggtcaattctttttttgggagcagtggtttgagattcggcaaaggtcgtctaaagtagtgagcgaacttttcattcgctccccaacacaaaagccgataagctagcatgtagttatcacattttaccgcgtaaatgagtaaatagtttatattgatacatgtaccatgttgctgtctgtttgggcatatgttgccgcattctgaacacttatgaatgagatcatagttcttacaacataaccccaaatgggttaatatgtctttgtcatgttttaaaaactcgatatgattctttaatgttttgagcgcaatgttaaataaactcagcattttaataaaatgaggtgttttagtattatgtttagtataaaatttagcggtatgtttgcatgatgctaaataaacttaacgttcctactaaaccaaaaaaaaatcaaattgactgactcatagagaatttgacgatgttggtaggtaattttttaacatggtatatatttttttaggatccgttatattaggtaataaaagaggacgtgccgttaaagtattttgcttaagatcctttaaatccttacaaaaatatagattgttcgtatgataatgccactgtgttacagtgatggcttgatcaatatcagctcccaagacaaaacagtagtatatcgttaaaaagttgtaatcttgcatacaagccaactgcatcattttatcgatgtccatatgaacgatcttttgctcgtatatttcatgaaggtcaaatacattgttgaagtaaatggcgcacatgagtcgccacatactaaggtgcccatatgtttgatagaaaaagaagatagctcttttaagcttatattttactgctatggcataccagtatttaacgaatatgttcatgggtacattatccaagatataaaatatgaaaagctttaactctcgatgaatctcctctcccatttcctgcacatttagagcttccaacataggatttttatcaaatatttcatgacataaaataatgttattactcgttttatgacgcattaaaccggcgaaaatttccttattatttaaaccatctttagctcctaactttcgacacagctcctgagtttgttccgtcctagcacaggtcagcccataataaatgtttgctccccattcggtgaacagccttattacatcatagttattttcttttatggccatgattaacgccacatcaagatgaagaagttcccccttaaagggggttgagcttaaaataacgtaattacagtagtgacataagctaatgggcttgttttgccaccataagccacaatattttaaaatataatgatactcctcaggcacgctctgtttggccacagcctttttggccagggtttgcaaggagagcatgataacttcttgaaaaaaaaactcaaattaagttcctacttttttaaaatattagtatggacagatctaccatcatatgaaggaattctttcatcgttaaacactgaagagataatactttcatcgtatagagaatatcatgtcaatccatatattgaatgttatatatcattaaacccatcattaatatagtgtttatgtgctatggacaggttttttgaatgataatcttttaacatacgttttataacttcgggatcagtttcttttaaagataaagaatcattcatgctataacaatttaatgataacatgctggcaatgaacgagttgtctttttggtgcgctagagtctttccctcctcaaaggcattggcgcctaagtctatacaaaagaatatgtttccgatattatagaactgaatagaatgaaacatggcctgattgatatcagcccctaaggcgacgcaacagtaataaatcgttaaatagttataattcttgcgacaggcccaccttagcatttcatccatgtccatacgaaccttctcctttgcgtacagctcgtgaaggtcgaacacattattgtaaaaaagggcgcacataagccgccaccgatgtagatgagcatatctctgataaaaatagcaaatcgcctccttaaggttaaattctattgccatcgcgtaccaatatttagtaaacatctcgcttaatatatcggtttctaccattaatccctccagttgttcataaatcattccctttacttcaaaacgatttatggtatctaaaatgggattattagaaaatacctcatggcagaaaatgatgttactgctagttagatcacgtttcaatgtgtaaaaaaatcgtaaaatttcctggtcatttaactgttctttggcacctagctgcctacacaggtctcgggtgtgctccgtgttgacagaaagcaaaccgtagttgatgtttgcgccccactcggtgaacaattctattagatcgtgattgttttcctccacagctttcaccaaggccgcgttaagatttgtgccgttcttaaaatacggcgtccatattttcttttgatgatacatgatagggccattatgccaccatagaccgcagcacttcaaaaaatgaggatggcatttggccggatactggctggccagcacctttttggtgagagtctgcagagagaggaccatatttctttttttgaaaaaatcaaattaaaaaaaaacatgcttgtttggcatacatgtaactatgttataaccatgttataacattgttataatgatggtataacatagttataacgatgtatcattgatgtcataattcaaataggccaacatactttttgatttatagttttttaatagacgatatattttgttaggatctgcttcttttaacgttaatagcgaggagtctggactataaatgtctaatgataaacgatgagatatcaaatagtaattctgttgctctgccaaggcttttgcctcttcaaaggcatcggcccccagatctatacaaaagaacaggttatccatattatagaatcgtatggaggcaatcatggccaaattaatattagctcctaagataaaacaataatatatagttaaaaaattgttatcttttgtgcaggctatccgcatcatttcatccatgtccatacggattttttctttttcgtacaaattatgtaggtcaaacagcttattatagcaaagagcacatgttaaccaccacgtattcagatacttaaaatgttggtaaacataagaaatggcctccctaagattatcctgcaatgccacgataaaacagtatatagttaacatatcaccatccgaaatattacttaatacgttggtatcttctgctaaattttttagcttccaatgtatacacgactttatttcccttataatgacataggctgaaaagggattgtcattaaaaaatttaagacataagataatattattactagtagtgttaggatgtattaatttaaagaatatgtgcataatcttctttttatccacttggtacttggctcctaattcccagcaaaattctcgaataggtggcgtattcgcgcaaattaacccatagttgatgtctgcgccccaatccgtaaacatttttattaattgatagttgttttcctttgtagccaacattagtgccgtattaaggtccaagccatctgcaaagtttggcagctttatcagcatatgtttgcaatcaagtgaaattggagccttataccaccatagcccgcagcgttctaagataacatggtaatcaatagatacatgctgactggctaatacctttttggcgaaggattgcaaggaaggaaccatcctgtttcttttttttaaaaatcaattatctttgttcataatcaagaaaaatccccatatttattgagtgataattttttaacatgtaatttattttttcagggtccataacgttcgacaacagagaaatgactggattgtaatgctttaatgataaggcatgggctatcagataattttccttttgttctgccaaagctttgccctcctcaaaggcgttggcgcccaggtctatacaaaagaacaggtttccaagattatagttttgtatggaaaccagcatggcttgattgatgttggctcccatgataaaacagtagtaaatggccgaatagctataatcttggatgcaggctatgtgcatcatttcatcgatatccatgcggaccctttctatttcatacagctcgtgaaggtcgaacacgttgttgtaaaaaagggcgcacatgagccgccaccgatgtaggcgcgggtatttctggtaaaagtagcgaatagcatctttgaggtcatagtccaccgctatagcataccagtatttggttaaaacagtgctaaagctatcatcatggtctagcatgaaggttatctccatgagccctcttaactcccacatgatttcccccctcagatccagattatctataatccttaaattggggttattggaaaacacctcgtggcaaaagataatattgctactggttttatcgcgcgtcgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaagcaggttgaccttataaatagtttattacgtcgaatatgaagcataatggggccattatgccaccacaggccacaacacttcaggatatgataatgatccaccgacacgcactgcccggccagtactttcttcgtgagggattgcagggaaggcaacatgcctttccatcctttgatggaaatcaaattatctattaataaccatcagtgtttatattaggtatttggatattatcccaggctggatacgtagtatcgctattcacatgtacttccaactctagccggcgcctgcaggtcatttatttttaatattgattcttttttgtatttaatcatttagagaaggtcatcataggagccagatgttctctctccagaacttatgtcgaaaaacattacctgactgtaaacttcctgaattttttgacgattatatattacaactgctgggattatactgggaaaaccatggaactattcaacgggcaggaaacaactgtgtgcttatacaacaacataccctcattcccgtaaatgaagccctaagaatagcagcatctgaagaaaattatgagatcgtgggccttttattagcgtgggaggggaacctttactatgctattataggggctctagagggtaaccgctacaacttaattcgtaaatatgatgatcaaatcaaggaccatcatgacattctgccattcattgatgatccaatcatatttcacaaatgccatatcatgcggcgatgcttttttgattgtattttatatcaagctgtaaaatatagtaagtttcgtgttcttctttattttaaatatacattagaggatgatttgcccctcgttcatttacttattgaaaaggcatgtgaagatcataattatgaagttattaaatggatatatgaaaacctacatgtctgccatataatagatacctttgactgtgctattgcccataaagatctacgtttatattgtttggggtatacatttatatacaacaggattgtaccctataagtatcatcatttagatattctcatactttcaagcctacaactcctacataaggtggcggccaaaggatacttagattttatcctagaaaccttaaagtatgatcataatatagataatttagatgttattctaacacaagctgcaacatataaccatagaaaaattttaacctattttattcctcaatcaacctacgcacaaatagaacaatgtttgttcgtggcgataaaaacaaaatcttccaagaaaaccttgaacttactactgtctcacctaaacctttccatcaaactcatccaaaaaatcagccaatatgttgccactttcaattcaacaaatataataggcattctgagtatgaagcggaaaaagaagatatatttggatatcatattgacaaaatttgtaaaaaatgctatttttaataaatttgtcgttcgatgtatggagagattttctataaacccggaaagaatcgtcaaaatggctgcgcgtataaataagatgatgttagtgaaaaaaatatctgaacatgtttggaaaaatcatgcggctagacttaaacaccttaaacatgcggtacacacgatgaagcataaagatgggaaaaatagactcatgaactttatctatgaacactgctattaccatatgcaaggggaagaaatttttagcctcgcaagattttatgcaatccatcatgcaccaaagttgttcgacgttttttataattgttgtatcctagatacgatacgattcaaaagccttcttttagattgttcacatatcataggtaaaaacgctcatgatgctactaatatcaacatcgtgaacaagtatattggcaacctgtttgctatgggagttcttagcaaaaaagaaatcttacaggactatccatccatctattctaaacattatatgccttagtttattttttgcggctgaaacatcattcttacctagaaaatgcttatagtcatcttaaatcataggaaaggaagatcatcatattttttgaaaagtaattttttaacgcatgatctatgatttcagggtccgtgcttttaggcaacgggatggtggtcggactataaatctttagagataaaatgttttttataagctcatacccttcccctaaagctgtagtaccctcttcgaaaacatcagcccccagatccatacaaaagaacatgttttctatattatagtactgtattgaggtaagcatggcctgattgatgttggcgcccaacatatagcagtagtacatggttgaaaggttgtggtctttgatgcaggcaatccgcatcatctcttctatgtccatatggatcttgtccttttcatacgcctcatggaggtcaaacacattattaaaacaaagagcacatgttaaccgccacgtattcaggtgtgtatatttttggtaaaaatactgtatggcctctttcaggctatagcgtacggctatagcgtaccagtatttgagtagtaatgtactgagcaaaaactcattatttagcagatcggttttttctattaactcccttaactcccagaaaatttctatcctcaatttcagattatttactttttgtaatatcggattgttggaaaacacctcatggcataaaataatgttactactagttttatgaaactttagatctatgaaaatttgtaaaatttcctcttcattcaagatctccttggcacctaactcttgacagaggtcccgggtatgctccgtgttgacagataccagcccatagttgatgtccgccccccactctgtaaatagttttataaggctgtagttgttttcccttacagcctttactaacgccgtatttaggtctaagccctctttaatgcctgatgattttatgagccttaggttatgatcaaacgtgatcggagcattataccaccatagatcataatattttaaaagataatgttggttcgtgggcacgcattgtccagccaacacctttttggtcaaagattgcagggaaggcaacatgtctcttcatcttttaaaaaaaaatcaaattaactagccgaataaaattttctttcgagggcttttttaaaaagaactctttaagagcttttaaaagcttttaaaaagctttttaagagattaaaaattatccttgctggcattttgccaagcatgcggcattcctatcatctataatatattatgagaatattcccaaatgatggataagttttttgatttataatcttttaataaactgcttattttttcggggtcctttaagtttaatggcaaggaagcatctgaactgtaaatatccaaagtcaagctatggcttaaaaaattataacctttttgttccgctatggcacgaccctcttcaaaggcattaccacccaaatctatacagaaaaatatattaccgacgttataatattgtactgaagtgagcatagcttggttgatgttgccccccagggcgtaacagtaatatattgttaatagattgttatccttggcacaagccagacatatcatgtcatggacgtctatttggacttttttcttgtggtacatctcatgaagctcatatattttgttataatacaggagacattttaatcgccactcgttaagatccgtatatttctcatctagaaagcaaatggcgttcttacaaacgtattgtactgctttggcataccaatacttcactagtaaatcatttaactcgtccgtttcttttatttctatgagcccccatagtcttttataaattaagccccttaattgtataacaaatttgttttctaacataggattattcataaaaatttcatggcacaaaataatactgccgctggttttattgtgcattatcctggtgaaaatacggaaaatatcgttgtcctctagagtttctttggcgcctagctgtctacacaactctcggatgtgcttcgtattgatagaaagcaaactatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacagtatttcaaaatgcagtgttcatctattgacaaacactggctggctaccgtctttttgacgagggtctgcagagagagcggcaacgacatgtttctttttcaccaaaaaaaaatcaaatgttctcgtctttaaaggttaattcatgttcttaaaatgttcattttcatgatagtgattaatatatggtttaataacgctagaaggcttgtttataagaagtctataagcagtctataagacagtctatgacagtctatgacttagtctataactataatttctggatgggctgtaagatactcttcggctcgtttcatattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcggcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctaatgcaaatctctttttcacgacacagctcgtgtatgtcaaaaatgttattaaaataaaggctacaagctacccgccaatagagctgatttttatgccttttataaaaatagtggatagcctttgtaaaattatgtcgtaatgccagggcaaaccaaaactttgttaataggaggtgcgccgtatcccccgtcaacggaatgtttgaacaggtgtacgtaactgtgtctaaagtggttctagttacggtttccaagagtggattatgacaaaacatgtcataacccagtagaactcctgcacaggattttaggttggccacttcttttaaaatttccagaagacggggttcggatacaggcgtcaagcctcccagttccgcacacagccgctttagatacacggcaggaacacgtataagcccatattcaggatttgcgccccaatccacaaataaacgtataagttcaagattatcgctcttcacggcctttactagcgccgcttcaagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacatgctaagtttaaaacatcttaaatctaagctagctaactttcaagaaaatcctctatccctaagaatatatcttataactagacttatagcagtaaaaatcaactttggttattctttttaatataaaacgtctaattacttgcaaagtattatcaagcccattttcctcagctagaatttttattttttaatgaagtagggggatatgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccctgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcagaccatgcatgtttacgacctaaagattatgtttaatattgccatctccaagagggatctgactatgtactccttaggatatattttcctttttgatagagggaacaccgaagctacgttgctaacgcaacatctcgagaagacagcggccaaagggctcctccactttgtgctagaaacgttaaaatacggcggcaacatagataccgtcctgacccaagccgtaaagtacaaccatagaaaacttttagattattttctgcgtcaactacctcgtaaacatattgaaaagcttttgttgctggctgttcaggaaaaggcttctaaaaaaacattgaacttactgttgtcacatttaaactactccgtgaaacgcatcaaaaaactgctgcgctatgtgatagagtatgagtccaccttggtgataaagattttgttaaaaaaaagagtaaacctgatagatgccatgttggaaaagatggtaagatatttttctgcgacgaaagtgaggacgatcatggatgagctttcgattagtccggaaagagtcattaagatggctatacagaaaatgagaacggatatcgtaatccatacttcttatgtttgggaggatgatctagaacgtcttactcgtcttaaaaatatggtatacaccataaagtacgaacatgggaaaaaaatgttaattaaagtcatgcacggcatatacaaaaacttattatacggcgaaagggaaaaagtcatgtttcatttagccaagctctatgttgctcaaaatgcggccacccaattcagagacatttgtaaggactgttacaaactggatgtggcacggtttaaaccgcggtttaagcaactaatattagactgtttagaaattgttactaaaaaatcttgctatagtatcctggaaatcttagaaaaacatattatttccctatttactatgaaagttatgactgaagaagaaaaaaacctatgtttagaaatattatataaagtaattcattataaaacaatacaatgttaaaattcaatagatatacatcattaatattgattatattttcgaatattatcttctatggtgcaagataatcatctagcgcgtgaaacatgtcctcttcccttcaggaactttgtcgaaaaaagctgcctgactgcatacttccagagttttttgacgactatgtattgcaactgttaggactgcactggcaagatcatggttcccttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagctctagaaagcaaatactatgacctggtttacaaatactatgacctggttaaagactgccatgatatcttgccgctgattcaaaatccggaaacatttgaaaaatgtcatgagttaaacaacccctgttctcttaaatgcttattcaagcatgctgtgatacatgacatgctgccgattcttcaaaaatatacatactttctggatgggtgggagtattgcaaccagatgctgttcgaactggcatgtagtaaaaaaaaatatgagatggttgtgtggatagagggagttctaggcatcggcaaagttacatctcttttcactattgcgattagcaacagagacctgcacctgtattccctgggccacttaatcattcttgagagaatgcagtcctgtggacaagaccccacgtttttactaaatcatttcctgcgagacgtttcaataaaagggcttctcccttttgtactgaaaaccatagaatatggtggaagcaaggagatagccataactctggctaaaaaatatcagcataaacatattttgaaatacttcgaaaccgggaaatgctaaatgcggtatgatgtacccaattgtaatgaatcgtatctttataaatgttgtaaaaacttaactatttgaccacatcatatgattttagaaatttcaagctgctgaacaacagttttatcatacattaaaattccagtaaaatttatattttttttggtaaacaaatgttttctctccaagacatctgtcggaaatatctttttcaacttcctgactcttttgatgaatatacgttacaagtgctagggctatactgggaaaaacacggatctcttcaacgaataagaaaggacgctgtgtttgtacagcgaaatctcatcatttctatcaatgaggctttaagaatcgcagcttcagagggcaacggtagggtagtaaaacttctgttatcatgggaaggaaattttcattatgtgatcataggagctctagagggtgaccattacgacctgattcataagtatggcagtcaaatcgaagactaccacatgattttatcatccatccataatgcaaacacctttgaaaaatgtcatgagttatccaattgtgatatgtggtgtcttatacagaatgctataaaatataatatgctccctattctccaaaaacacagaaatattctgacacatgagggagagaatcaggaattgtttgagatggcatgtgaggaacagaaatatgacatagttttatggataggacaaaccctaatgttaaatgagccggagtttatttttgatatcgccttcgaacggatagatttttctttattaacaatgggttatagccttctttttaataacaagatgagtagtatagacattcatgatgaagaagatcttatttcattactaacagaacacctcgaaaaagcagccactaagggatgtttcttctttatgctagaaactttaaaacatggtggaaatgtaaatatggcagtcttatctaaagctgttgagtataatcatagaaaaattctagactattttatacggcaaaaatgtttatcacgcaaagatattgaaaaactattattagtagccatatccaatagtgcatccaaaaaaactttaaacttactcttgtcttacctaaaccattccgtaaaaaatatcattggaaaaatagtacaatctgttttaaaaaatggtgattttaccatcataatatttttaaaaaaaaagaaaataaacctagtggaacctgctttgataggttttataaattattactatagctattgttttttagaacaatttatccatgagtttgacattcgtccggaaaaaatgattaaaatggccgcgcgaaaaggtaagctaaatatgatcatcgaatttttaaatgaaaaatatgttcataaagatgatcttggagccatatttaaatttctcaaaaacctagtatgtaccatgaaacataaaaaaggaaaagagacattaattgttcttattcataaaatatatcaagttattcaactggagactaaagaaaaatttaaattattaagattttacgtcatgcatgatgcaactatccaatttatatctatgtacaaagactgttttaatttagccggttttaaaccatttcttttagaatgtttggatattgctattaaaaaaaattaccctgatatgatacgaaatatagaaactctgttgaaatgtgagtaaaatttatttttttgatcagagtaagaaaatgttctccctccaggagatctgtcgaaagaacatctactttctacctgactggctcagtgagcatgtgattcagcgactaggtctgtactgggaaaaacatggttctcttcagcgaatcggagacgactatgtacttatacaacaggatctcatcatccccatcaatgaagctctaagaatggcaggggaggaggggaatgatgaggtggtacaactcctattactatgggagggaaacattcattatgccatcataggagctttggagagtgaccattatagcctaatacgtaagctctatgaccaaatcgaagactgtcacgacatccttcccttaattcaagacccaaaaatctttgaaaaatgccatgaattagataaattttgtaacattttatgtctcgtattacacgccgtaaaaaacgatatgctttgcattcttcaagagtataaaatgcatctaagtggagaggatattcaagtggtgtttgaaacagcatgccgttcacaaaaaaacgatattgtgtcatggatgggacaaaatattgcaatatacaactccggagttatttttgatattgcctttgataagatgaatgtgtccttattatctatagggtacacgcttcttttcaatcatcatataaataatacgaacgaaaatattaattctttattgacacaacatcttgaatgggctgccggcatgggccttcttcattttatgctggaaactttaaagtatggcggggatgtaacgataatagttttgtctgaggccgtaaaatatgaccacagaaagattttagattattttctccgtcgaaaaaacttgtaccaagaagatcttgaagaactattattgttggcgatacgtgcagattgttctaaaaagaccttaaacttgttattatcttacttaaactattccataaacaatatccgtaaaaaaatattacaatgtgtaaaagaatatgaaacgaccgttattataaaaattctatggaaaagaaagataaatctgatagagcccattttggcagactttataggatatcatagctatacctatatggtagattttatgcgcgagttttccatccatccggaaaaaatgatcaaaatggctgcgcgagaatcgagggaggacttaatcataaaattttccaaaaaagtttgcaaagagcctaaagatagacttcactatctcaaaagcttagtgtatactatgcgacataaagaaggcaaacaactgttaatttatacaatccataacttatacaaagcttgtcatctagagagtaaagaaatgtttaatttggcacgattttatgcacggcataatgcagtgatccagttcaaatcgatttgtcacgatctctccaagctgaatattaatatcaaaaacttgttgttagaatgtttaggtattgctattaaaaaaaattactttcaacttatcaaaacaatagaaacggatatgcgttatgagtaaaatttttggataagggaagattctaccaaactaactaagacctttcgctagaatgtatcttattgttaatatagattaggtatgacgtgaaaaaatagattaggtaggttgtgaaaaacagattaaacttaaaattatgtgtattatgtaaaattttagaaataaaaatttatttttttttattgagggtacggaaaatgttctccctacaggacctctgtcggaagaacactttcttccttccaaatgattttagcaagcatactctacaacggctggggttgtattggaaagagcatggatccgtccatcgaatagaaaaggacagcataatgatacagaatgaattggttctttctatcaatgatgctttacagcttgcaggagaggagggggacacagatgtggtacagctcttgttactatgggagggaaatctgcattatgccatcataggagctttgaagactgagaattataatctagtatgtgagtaccatagccaaattcaggactggcatattctcctacccttgattcaagatccagaaacattcgaaaaatgtcatgatttaagccttggatgtgaccttatatgccttctccaacatgctgtaaaatgtgatatgctttctattcttgttaaatataaggaggatttactaaatgtaaggattaggcatcgtacccaatccctgtttgttttggcatgcgaaaatcggagatttgagattattgaatggataggtcaaaatctgtcaattcctgaacctgaggccatttttagcattgctattgttacaaaagatgtagaactgttttccttaggatataaaattatttttgattacatgcaaagacaaggaatttttcaattaaccaatgtagttcgcatgcttctgctaaatcgtcatattggtatggcaatagaaaaaggacttttaccttttattctggaaactttaaaatatggtggtagtgtaaaaagagctttatcttatgcagtaatagataataaaagaaaaattatagactatcttgtacgccatgaaaatataccccgaggaactattgaaagacttttgcatctagctgtgaaaaaacaatcttccaggaaaactttgaacttgttgctatcttacataaattacaaggtgaaaaatgttaaaaagctggtagagcatgtagtagatcacaaatccactcttgtgttaaaaattttattggaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaaggcggtttttgatagacatcattcacagcatttaccaaagttactcactaaaacacgaagatattcttaaactggcaacattttatgtcaaacacaatgcaatcacccattttaaagatctctgcaaatatctttggctgaacagaggaacagaaagtaagaaactgtttttagagtgcttggaaattgctgatgagaaggagtttcctgatattaaaagtattgtgagtgaatatattaactacttgtttactgcaggagctattaccaaggaagaaatcatgcaagcttatgctttagagtatgacatgtattaaatttctgaaggtagactaaaatatactatatattaaaaaatccaaaacagccatttttaactaacttcttcttaaaaactctggataaaaatttatttttttttaatttgagtagggaaaatgttctcccttcaggacctctgtcggaagaacaccttcttccttccagataattttagcaagcataccctgtatttgctggggttatactggaagggacatggatctatccaaagaacaatgaatgatggtgtactgatagagcataatcttaatctttccatcaatgaagccttaatccttgcaggagaagagggaaacaatgatgtagtacaactcttattgctatgggaaggaaatcttcattatgccatcataggagctttgaagactgagaaatatggcttaatatgtgagtaccatagccaaattcaggactggcatgttctcctccccttgattcaagatccagaaacattcgaaaaatgtcatgatttaagccttgaatgtgatctttcatgccttctccaacatgctgtaaaatataacatgctttctattcttgttaaatataaagaggatctattaaatgtactatttaggcaacaaattcaaggactatttattttagcatgtgaacatcggaggattgagattcttacgtggatgggtcaaaatctgccaattcctgatcctgagcctatttttagcattgctgttgtcacaaaagatttagaaatgttttccttagggtacaagattgtttttgaatacatggaaaatcaaggactatttcatttaacccaggtagttcgtatggttatgctaaatcatcaccttggcatggtaataaataaaggacttttaccctttgtgctggaaactttaaaacatggtgggaatgtaaatagagccttatcttatgctgtcacacaaaacaaaagaaagattttagaccatgttgttcgccaaaagaatataccccataaaaccattgaaagaatgttgcatctggctgtaaaaaagcatgctcccaggaaaactctgaacttgttactatcttacataaattacaaggtgaaaaatgttaaaaagttgttagaacatgtagtgaaatacaactctactcttgtgataagaatcttgttagaaaaaaagaaaaacctgctggatgctactttgacaagatatgtcaaagattctacatactttcaggtgaaagaatttatgcaagacttctccatcagcccagaaaaattcattaaaatagctgtgcgggaaaagaggaatgtgttgatcaagggtatttctgaagatatttgggaaaatcccgcggaaagaatcaggaatcttaagcagatagtgtgtaccataaaatatgaaagtggaagacaattcctgataaatatcattcacaccatttaccagagttattctttgaaacctgaagaaattcttaaactggcaacatttatgtcaaacacaatgcaaccacccattttaaagatctctgcaaatatctttggctgaacagaggaacagaaagtaagaaactgtttttagagtgcttggaaattgctgatgagaaggagtttcctgatattaaaagtattgtgagtgaatatattaactacttgtttactgcaggagctattaccaaggaagaaatcatgcaagtctatgctttggagtatgccatgtattaaatttctgaataagtaagcaatagataggttttagaatatgctgtattaagttagtttctgaataagtaattaatagatagattttagtttatgtaaaaatgttaacatttgttaataagttttagataattactattttagagttactattttagattttaccattttagctattattatcttaaataatcactattttagataggtccccgtattaaaaaccaaattaaccattatctatgtttttaataatactttttaaaaaccctccataaaaatttatttttttttcataaaagtagagaaaatgttctccctacaggatctctgtcggaagaacctttttcttccacttgagcccttaggcaagcatgtggttcaacggctgggattatactgggaaggccatggttcacttaaacgagtgggtgattgctttatatgtgtagacaagatttggatcctatccatccataaggctatacaaattgcagcctcggaaggaaatgagaacattgtcaagcttttcttactgtggaaggggagtctacaatatgccatcataggagccttagagggcaggcaatatgatctgattcaaaaatattacaaccaaattggggactgccatgagattctaccactgattcaagatccagaaatttacgaaagatgccatgaattaaatgttacatgtacctttcaatgcttatttcaacatgctataagagataacatgctgcccattttccaaaaatatggagaagatctgaatggaaacagaagaatggttcaacttctatatgaaatggcatgccgattacaaaattatgatatcatcaaatggataggatttaacctgcatgtttataacttggaagccatttttagcattgcttttgttagaaaggatttaactttgtattctttaggctacatgcttcttctgggtagaatgagtactgaagatagaaactttatttcaatcataacacgccatcttgaatacgcatcaaaaaagggactttttgactttgtactagaatctttgaaatacggaggtcaagtggatacagtgttgtttcaggctgtaaaatacaaccatagaaaaattttggcccattttattcatgaaattccgcgtgaaacagttgaaaagctgatactccatgctgtagaatcgcgggcctccaggaaaacattcaacctgcttttatcttccataaactactgtgtaaacccttttgtcaaaaaactactgcacaccgtggtgaaacacaagtacatgcttatcataaagcttttgctcgagcggcccaaaaagaagataaacctggtagatgctgctctattcaaacttgtaaaatactctacttatgcagaaatagtaaaattcatgaaagagttttctgtggacccagaaagggtggtcaaaatggcagcacgactcatgagagtggacctgattaaaaagatttctaacgatgcatgggaaaataaactagagagaatcaagcaccttaaacagatggtaaataccatgaaccacagaaatggaaaaaatctattgatgtacaatattcacaatattactggatatacctgcttgaacaccaaagaagcatttaacttaacaagattttatgctgtccacaatgcaacatgtttgtttaaagaaatgtgtaaaagctgttttgtacatgataaaatacagttcagagaattgcttgaagattgtttacatattgctaataggcatgattatatccagattgcagaaaccgcagatgaatgtatcaaatatatagatcttattacacctaagtaaatcatgaaaatatatcaagtaaatccagattaaatcaggctaattgtaaatagttgtagataccatataatgaatgttttattaggatagtagttaatagtttagttaagacagtagttctttctgttaagatagtagttctgttaagatagtagtttagttatgatagtggtttagttaagacaatagttttgttaagacagtagttctgttaagtcaatagttcagttaagtcaatagttttgttaagtcaatagttctgctaatacattagttctgttaagataataaaaatttatttttttttcatcaaggtagagaaaatgttctcccttcaggagctctgccggaagaacatttacattcttccttaccccttgggtaagcatgtacttcaacaactagggctgtactggaagggacatggatctcttcaacgaatcggagatgaccatgtactcttacaacaggacctgatcttttccatcaacgaggccttaagaatggcggcagaagaaggaaacaatgaagtagtaaagctcttgttactgtgggagggaaaccttcattatgccatcataggagctttagagggcgaccgatacgaccttatccataaatattatgaacaaattggggactgccacaagattcttcctttaatccaagacccgcaaatctttgaaaaatgccatgaattgagtaactcctgtaacattcgatgccttttagaacatgcagtaaaacacaacatgctttctattcttcaaaaacataaggatcaaataagattacacatggcattaacccaaatactatttgaattggcgtgccatgaacgtaagaatgacatcattagatggatcggttattccctgcacatataccatctagagactatttttgatgttgcattcgcccataaaaatttatccttatacgttttagggtatgaacttctcatgcacaaagtaaatacagaggctgcaaatatagatttacccaatttgctatcatatcaccttcgaactgcggcggcaggaggtcttcttaattttatgttagaaacaataaagcatggtgggtgtgtggataaaaccgttctatccgcggctatcaggtacaagcataggaaaattgtggctcattttattcatcaggttccccgtaaaaccgttaaaaaactgctactctatgctgtgcaggctcgggcccccaaaaaaacactaaatctacttttatcttccttaaactatgccgtgcacaccatcaccaaacaactcgtacacaatgtcatcaactacagttccacgcttgtcgtaaagcttttactcatgcggcgaaaaaggaagttaaacctagtagatgccgttttagccagacttgtaaaatattccacctatacagacattgtacaattcatgggtgagttttctgtgagcccagaaagggtgatcaaaatggctgcacgggaatccaggacctttctgattgaaatgatctccaaagctgcttggggaaatcacccacagacgttgattcatcatctcaaacacctaaccaataccatgaagcctcaatctggaaaagacctcatcatatataccatccactatatttatctaaactctaatatgctggtagcggaggaggaaaaaaatatttttaaattagcaaaattttatgcgaatcataacgcggtaaacaggtttaaacaaatttgtgaagactattatatattagatgcacgatttaaaacacttattttagaatgttttgaaattgccgtccagaaaaactatcctagaattgcaaatattgtggatgactatattcgattccttttttacaggggaaatataaccgaggaagaaattcgtgaagcctattctttaaaagatgctgaggtttatgtagatttaaaatggttacaacaaggagaaatggtttaaactaaatccggtttaaactaaattcggtttaaactacatttggtttatcattagtcattgaaaccatcgaaaaaaaagtatttgtttatccccataaactcatcttttttttgtctcaaagtttgacactaaaattcagtgttttatagtgtttataattaagtgttttgcatgcattgcagaaattttcatcttttttaattggttcaataccacatgtcatacaatatgttgtttgattatcaagattaactttatgaaaggaaagtaagtgagccgcaaatttaaaagtaaaatatctttcatttaaaatgatcttatgaatgtattttcgataaggaggaataaaagcatttgccaaaataaatcgcataaagggcttggaaaaacccatatcttctaatcttttgtgggtataaaccctatttttgtgttttacaaaaacttcattgttataatagtcgttatagctatcaatcatttttttaagtcctataatgcccaaggttgcacgcataaagccgcagtttctactccaaaagccatgcacctgtaaagggtgcttttcatataaccaattacaaaatttcattccgcaacagtagcatgttatttcagtgggggatgtatagaaaaatccggcattcgaaaatttttcataattttttatgccatggattgcgaagctttgatttcgtgcatctatggagctatagcctacatatttaggttttacttcaaataatcgcaaagagatgtatggatctattgtatttattttaggaaacatttcataattttaaattcttatatataatataaaaaaaattacaaacatttgtaatgatcatcctcaattgaaagctgagctgcaggctttatttttctaattatacgaagaaggtaggttctcataaagccttcaagatgactattgatgtttccaatacattttctcaatgagttcataaacccagacattttgctaatggcttggcaaagtgccaacaagttgtccacaaagtactggtagattgcctactagctatagctagctatagtgagccaacctctctgtatttattttatatatttcattttttaatagatttaatatttttataaaaaaatatttagttttttatacaagaatgtcgacaaaaaaaaagcccacaattaccaagcaagagctttactccttagtagcggcagatacccagttaaataaagcattgattgaaagaatctttacaagccagcaaaaaataatccaaaatgctttaaagcacaatcaagaagttattataccacccggaatcaagttcaccgtcgttacagtgaaagctaaacctgctcgccagggccataaccccgcaacaggagagcctattcaaattaaagccaagcctgaacataaagccgtaaagatacgagcattgaaacctgttcatgatatgttaaattaaataataaagccatcatcatcgtcgtattcttctttatcattattatcttcaatacatttttgccaatcgaaatcgaataaattcagatcctagacatttaaatacttatcatcgtacattttaatataatttaaacatgagttgttgtcaaaaacttttatcgtttttgttaaaatcattgtatgaataatttccttattaagagttgccggaataatacaaagcctatttttaggtacatcattcatgataatagtaaaattagtaaaaattgtttcttgtttttcttttgtttcaaataaacgttgtaaggttaaaggtttttcattcaatggtttctttgaagataaaaagaatgtataatccggtttaaagatatttttggtttcaatcgtgattccatctgcttgagcatatgctaaaccagaccaaatataatggtccactattacaataaaatttagtttaagtagtgctgcaatttctgcgctaaattcactatgatgttttgtaaataatttatgcaattgttccgatgacatttctgtcattttatttaacacctgcaatataaggccaccggtggtcgtgtctggattaggaaaacgtatacatacagcattataatccatgcattctaatgccttttttaatctcattgcctgtgtgctttttcccacaccattgattccctcgatggtaataagtattccacgcatgattaataaatgggtaaaaagagttcagtttttaacatttcttacaaatctatttttatacaacattgtacaacactacattaaccgcatacgatgttatagcttcattaaatatttgcttttatataatctttaccgacctatatttggcagatcactgcagatggccataactaagataaaaattatttcagatgctactgcggcagtattattaaaatcatgtgcggcaatgtatgacgtcttaatagataaaaaatttaagtaaaacaaatttgaataaaaaaaataatagttatgatggcgttgttacacaaagaaaagcttatagagtgcatcgaaaatgaggtgcttagcggtggtacagtattgctcctagtaaagaatattgttgtgtcagaaatttcatacattgacaatagttataaatattttacctttaatgccaatcatgatctgaaaagcaaagaagatcttaaaggagcaacatccaacaacattgctaaaatgatttataattggattataaaaaatcctcaaaataataagatttggagtggtgagccgcgcactcaaatttattttgaaaatgatttatatcatacgaattataatcatgaatgtataaaagatttttgggatgtttcaacctcagtcggtccttgtatctttaatgatcgtagcatttggtgtactaaatgcacatccttttacccatttaccaacattatgtcgcccaatatattccaataaattagatatcttttactactattaaaatagttaataaccttataggataattaggtactttattacgatgaattagtttatgttattttataattaggtactttattacgatgaattagtttatgttattttataattagttactttattacgatgaatctttattaacgattcttttattaatgaattatcataagataaataattatttttttctccatatatcgcataataggtctggtatgggctaaaagtatgtttcaaactatttacaatagaatttctgttaagaaaacatacataatttgaataaaatttttttaaatatcaccgaaacatccaacatggtgttaatagagtttttaacaggtttcttctatttatatggaaagagactgttttccattagcaaagtcatggacatgatatgtctagactattataccattattcctgctcctctggcgatgatgttagcggcaagaataaaaaactatgacctcatgaaacgactgcacgaatgggaaatctctgttgactacgctctacttgtagtagatgatgtgccgagtatcgacttttgcttaagtcttggcgctaaatccccgactagagcacaaaaaagacaactgttgagggacaacacgtttaatcccgtgtataagtatcttatgaactgttccggttttccaacaagaagagaaaaaaacattccttgtgatgttcaatgcgaaagactgcaaaaaaacattataaaagaactggtattcaactgctccgtgctgcttgaaatggtactgcacacagaaagagaatatgcatacgccctacactgtgctgcaaaacataaccaattgcccatcctcatgtattgttggcaacaatccacagacgcggaatccattttgttgaaaacctgctgttctgataagaacatcaattgttttaattattgtattctatatggcggcgcccagaatttgaatgctgcaatggtggaagcggcaaagcatgatgcccggatgctgataaactactgtgtcatgcttggtggaagatccttaaacgaagcaaaagaaacggctgccatgtttggacacattgaatgcgcacaacactgttttgaactgcagtcttatgtcatggacgcattgaatgcggacgacgctgattaaagcgacaatcttacgtcatgaatgactgtcttttgagtatctatacttacattatatttttttatgaaaaaaatataaaggttgtatacaaaggtatacaagaaatttggatcattaaacaataattaatttagacacaggaaaacgatctagatcgatcaaaaagctatttttttgcacacagaatatttagataattaagagattactttccatacttgttcagtttttttacacacaggaagtttggattctgttcaggaagtttttcatagacattatttttacagccagtaataataattttgggctttttcttaaaccaccggtggaaaacatccagcttgtaaagagggaaatgcatgtagagaggtttttggtagtcatggttaagagatttgactaactccatatttcctgtaaagactgcccagtcccaagcagtaaagcctctatgatagtctttttgagtcggatctgctccaaattttatgagagaaagcacatttgaagtacggccccgtattgcggccttcatcacaggagtcatcccattaaaattcggtaaacaaattttggtcccattttttccgaaatagcccaacacctcttccaggattaaatgattttttttctcagctaaataatgtaaagcagagtttccatctttatccctcctatgagggttaattatttctccgggataagattcttgttcaaaaagaaattttaaaaagtctatacgtccgtagatgcatatccacatgaataccgaggatccatttttatcgcatctattgacaatccacggatctgttttaaaaaattcctcaaatagtgtaagatttccatttctgatatgttttttaatccatttaacaaacaagttttctatctccctttctggaaacatgtgttccattttgaatgtcgccccaccccattatataattttactcctttaatttttaatgtccttttttttcggacttctttggataagctgtttattaccatctttaaatgccttatagcggggaggagccaggccgctttcccatatgtgcggtaattcttggtgtttatgcttgcctttggcatatccaggccagtatttttcgatatattcgggatttgtttttacatattctttaaaggtccgataggcttcttgaatacaggtaggctcgcctgtataatttccgtgttcatcttcctttaaaaagccattaaccctgtcctttctccacttaagattgtgctttccaaaaatacggtcaagatcttcggcctgctggggtgggatcataaacccctttttaggtcgaagctttttattttttccatagcttcggccatcgcgttgcgaaacagtggttaggacgcccgacagcctttccatgggcgtcgcgtccaatcctatccatccaccctgatgaatatcaatggcaacaagctctcctttattttgggcaagccaagtttccaagaatgccatgctttcttcccagggataaggcccgccaacaccacgggttgtccaatcttgcaaggactccaggtccgacacctggtaaggctctaaagaagacggttccttgtttttgtactgcaaataggatttaatgacccatttataccatgtgtcgaaccgcagcgtggcgcctccaaagtgaaagccgtcgttgattttaggatatctgcaacatatttcaaccgtacgtttgagttctgcaaaggcggccttccaaggaagtctctcgctgcgggtaaggcggtctattttgccctgcgtaccatagcgtatggcgtgacgtgccaattgcaacaattctgacaccgatccgtgggccccgatccagtttatcggataggcaacctctgaagggtttaaaagatgctcgtaaaagcgtggatcttcagacgccaaggcgtctgcaaaagggataatactagaaaacctgtctaggcatacgttttctgtgtttacttctaaaggtagaaaaatggttgcgtgaggcttttgaacctgcttgttcagcggtctgcatatgctttgaataatgtctctaggactatgacgcggcgctgcaaaaaataccgcgtttagttccggaacctctacgccctcttgaaagagtcgacagtttaataaaataacgggttcctttgaggaacaaaattctgtaaatgttttgaggataacctgtcgcggcagggttgagtgagctatcagggcatagaccccttggtctaccaactccgcgtatagctccctggcctgtttaatatcacgggtgaataccagcattttaggagccggtatattggtttttaaataggctaaggccattataatttgctttactatgatctgtttcgtggtctcctctctgttactcggttggtgagccaatttgggcgcggctaccatttgtaattcaaaatcatttacatagccggcctctatgccttctcgcagatagtagcgaaaggcaacgccgccaaaaagttcacgatttttcatggaaagcggggtgtcgtacctgggcgttgccgttaaaaaaagtcggtgcccttttttaaagttgagcaacacatgggtaaagggccgtgtctcccattcaccgcaaatccggtgacattcatcgctaataataagatcgaaatcatccaccagtagcgtggaggattggtaggtggcaatcacaagaagagaaggggcctcccgtatccgttttgcaataaagacaggattggtggtcatttctatattgtcgtgatttagcacaatgcgggtctggtcagaccccacaagcaaaacgttcttcaaagaaattccatactgatagagtttttccagagtctgccgtagtagggacaggcccggcaccaggtacaaaacttttccttgaagataattggagaggataagataggcgacgcgagttttgccgcagcggcaggccatctgcagaatggcccttccacttcgccgcagctcctgatagcccatattggccgcctccttctgataaagccgatcctcgattgcagtccgtgtctcatctgtagaaaaaaataatacgtcatctgcgaaatgttcttcatcttccacaggagttatcaccaggtgtctcagtttctccttgcttatcagcggatcagagggcaaagatggctcaaccactatcgtggaatcattcatctcataagtgggagaatcacacaaagtatagcttatgtccagacagtttgcaacatcctcagccaattgttttattttttcgggtaaaagacatacgagttctttgtttttgacgcgaaaaaactgtgcgcaatacaacacccctgcttcaattttttgcgcatccttctttgtagatgtttccaatgtgaaacaatacttccattcatccgtaaaacaggttgtataagatccatcatggagcctagcggccaagtttcctgtgtgcccaactttatgtaaggattgggcctccagccagggatgaaccgccacgtaaaatcctgcgcacatgctatatcaaattgcagtttcttaataactgtacacaggatctgaaaaacatgtgattacaaaatttagataagaaatatttaatattaaaaatcacggaatacatgtcactgtgtagagagaaagccaaaaactcctcttgaccgccgtgggaaatcatccagggtagtaggttgtgtttcatgaagttgtatgccgtagtgatcaccgtggactccagatggttattggcgtctttgcaatactttgccatcttggcagaaaagacgataaatccacaaattctaccccagttgataagatccttaaacagctcagtcacaaccccagtaaactgggttttaatttcttggacactcgtaagagaaaaggtaattgtaacctgtttgttcaaacactcatcataataggttaaaattttttttatttgttgttgatatgggctaagctcatgctctgaaatatcattaatgtaatatttaatataacccactagtatttcattaatgatattatgatatattaactcttctccctccatagcggcaccctatatttttttatttaggtttcaatgttatcacaattgcgatacaattgtgatacaattgtgatacaattgtgacacgactgtgttgtatacaacaaatgttaggccacgtatagcaacctatatgttaagaaatatttttatcccaacattggttggaaacgagcagccgcaaagaagtcatttaaaataagccatttaaagatttagaatttatatgtatacaactgtacaatggaagcagttcttaccaaactcgaccaggaggaaaaaaaggctctccaaaattttcatcgttgtgcttgggaagaaactaaaaatattataaacgattttcttgaaatccctgaggaacgatgcacctataaattcaactcatacacaaaaaaaatggagcttttatttacccccgaattccacaccgcctggcatgaagttcctgagtgcagagagttcatattaaactttttgagactcatttcgggacatcgagtggtattaaaaggccctacatttgtttttacaaaagagaccaagaacctgggcattcctagtaccatcaatgttgactttcaggccaacattgaaaatatggatgatctacagaagggaaatctcatcggcaagatgaatatcaaagaaggctaaataaaacaactaacatcaaaaaacattaaaggctatgttgtggacgatgcctttgtctcaatagtttcgaggtcatccaataactcatgtaacgtaaaaaagttggtccatttttttgaaaacattaaaagacgttcgtcttcataaataaaaaagtcattcgaaggaaaaatgatatactcaataccatagtcttgtaatattttttttaggtctctcagggtccagggatttaccaggcttctacgcgaagtgagcatcataaaaatatctaatattttttgcgccatgagccagcgcggattctcattggcccacaaatcaacaataattctcttatcaaccgtgagcattcctacttgattcgaagaaatgattagatgcccggcggtccaccccataagtagataacgcagcgttgtagaaatgtcacatatggaaggcattcctccacaacatgaacccaaattaggatgcgtgtgaaacacaaacatagcaggcttgttggccaccctgctataaatatcagcaggcatcatagcctcgctgccaaaataaatgttctctcctgccctataggggcttggaatgatttccactatctcgggtacaccgtttatcatattaatgcggccgcaccattcacggtcatcgtccaaaaattttttgatggcaccccgaacattgtcccaattaagcaacagagtgttcacaatctcattacgctccgcccagtattccttgaaacttcttttagacttgctgagctgttcccaggattcgaactcggtccaatgttttttttcttttggggaagacttcccttttgaaacattttttgcggctccaccatctacaccatgattttccaaaataatctccttcatcgtttgagttatatgggcattgctaagcaccttagtggtaacctgtttacctatgtgatttagcagaaaaccaagtttgtccatttgtgtctcaaccatttattcttaacaaaacaaaaaaaattaaaaatcatcgtcgtttaaaaagagtttgaaggcaaacgcatcatccttaacacagttctgatactgcgtaggtcttaactcgaaaaagttggttttttctacttcattaagaaagaatttagtcatctgaggaaaagggtttcccactttataaatacttttgcactgcatcatgaagcacaaattatctgtaaagtagcgtatatattgaaacagcatttcttttgaaaaaccgggaactcttcctcttgccttgtcaaaggcatagttaatgaactcatccaccaactccacagcctccttcaaaattttgtgaatgatcttttcctcgggaatgttatacacgtaattcgagataagaaaacacgcaaaactacagtgcatcccctcatcgcgtgaaataaactcattatagcttacaagccccggcataatattctgttccttaagaaactggatcgccacaaagtggttttgaaataaaatgccttctacggcggcgaagcccaccagccgctcacccagagtgttcctgtcggggtccatccactgccgcacccactgcgccattttttttatgatagggtgtttttcaatgccgctaaagatgcgctgttgttccttctcatccgggatcagcgtttttacctgtattgagtaggcttcactatgaacacactcttgggcagcctgcattgtataaaagtataacacttcctttactttaatttcgcgcataaaattggttaaaaggttttcgataacaatttcgtcggcaacaacaaagaaggctaaaatttgtttataaaattcgcgctgtggctttggcatggcttcccaatcatcaatgtccttacacatgtctacctcctgcgccgtccacgtcaaactttctaattttttataccagttccaacattcggggtgctgaataggaaaaatagtgaaacgttgggaattttcaattagtaattcctccatatttgaaataaatattaacatcttcaaatttattggctgccatggagacgttttttattgagacgttggcatctgatgtgtatggaaaggcgttgaatgttgatttagatagactatcgcaggcgcaggttaaatatacccttcaagagcttatttcctactgcagcgctctaaccattttacattatgactattcaacccttgcggcgcgactttcggtgtaccagctgcaccagtcaacggcctcctccttctcaaaggcggtgaggctgcaggccgcacaatcctgctcacgcctgtccccccagtttgtggacgtcgtttacaagtacaaagccatttttgacagctacattgactataacagagattacaagctgtccctcctggggatagaaaccatgaaaaattcctatttgttaaaaaataaagatggggtcatcatggaacgcccgcaggacgcctatatgcgcgtcgccattatgatctatggaatgggaaaagtagttaatataaagatgattctgctgacctatgacctgctttcccggcacgtcatcacacacgcgtcgcccaccatgttcaatgcaggcaccaaaaagccgcagctttccagctgctttctgctaaatgtaaatgataatttagagaatttatacgatatggtcaaaacggccggcatcatttcaggcggcggcggtggaatagggctgtgcttgtcaggaatacgcgcaaaaaatagctttatttctggcagtggtcttagaagcaacggcattcaaaactatattatgctacaaaatgcctcacaatgctacgcgaaccagggaggcctacgccctggggcctatgcggtctacttagaactatggcaccaagacatctttacatttctgcagatgccccgcttgaaaggacaaatggctgaacagaggcttaatgcccccaatctcaagtacggtctatgggtccccgacctatttatggaaatacttgaagaccaaatacataacagaggcgacggcacctggtacctcttttcaccggatcaggcccccaatctacataaggtttttgatttggaacggtcacagcacgaaaacgcacatcgcgaatttaaaaagctttactatcagtatgttgctgaaaaaaggtacaccggtgttacgacggccaaggaaatcattaaagagtggttcaaaacagttatccaggtgggaaacccctatatcggatttaaagatgccataaatcgtaaaagtaatctttcacatgtaggcactatcacgaactccaatctttgtattgaagtcacaatcccctgctgggagggtgataaggctgaacaaggcgtttgtaacctggccgcggtaaatctagccgcctttatacgtgaaaatggctatgactaccgtgggctcatagaagcatcaggcaacgtcacagaaaatttagataatattatagataatggctactaccccacagaagccacgcggagaagcaatatgcgtcaccgacctattggcatcggggtctttggcctagccgacgtgtttgcgtctttaaaaatgaaatttggttcacccgaggccattgccatggatgaggccatccatgcggccctatactacggggccatgcgacgatccatagaacttgcaaaagaaaaaggaagtcatcccagctttccggggtctgcggcctcaaagggtctactgcaacccgacctatgggttcgctgcggtgatttaagttcctcctgggaagaacgcgtggcacagacgacgcagggtgtgttaacgcggaaaagctggtggcaactacggctggcggccatgcagggagttcgaaatggatatctcacggcccttatgcccaccgcaacctcctcaaattctacaggaaaaaacgaatgttttgagccctttacctccaatctatatacacgtagaacgttaagcggggagtttattgttttaaataagtatttaatagacgatttaaaagaaattgatctttggacagaagccattcaacagcagctactaaatgcaggaggtagcattcagcacattttggatataccggccgagatccgtgatcggtataaaacctctagggaaatgaatcaaaaaattttaacaaaacacgcggccgcacgaaacccctttgtgtcccaaagtatgtctttgaactactacttttatgaacctgaactaagccaggtacttacggtgctcgtcctaggctggaaaaaaggtctaaccaccggttcctattactgtcattttagccctggagcgggtacccaaaaaaagattataagaaactctgagaaagcgtgtaatgcggactgcgaggcgtgtcttctgtaggagtctcgcggtaaaagagcagcggggaccatatggcaaaccccaataagaggataatgaataaaaaaagtaaacaggcatccattagttccatattaaatttttttttcttctatataatggaatattttgttgcggtagacaatgaaacccccttgggggtttttacttctatagagcaatgtgaagaaacgatgaaacaataccccggcctccattatgtcgtttttaagtatacttgtccggcggatgcagaaaatacagatgttgtatatttaataccctcgttaaccttgcacacccccatgtttgtagaccactgcccaaatcgtaccaaacaagcacgacacgtattgaaaaaaataaacttagtgttcgaggaagagtctattgaaaattggaaggtttcagtaaatactgtattcccccatgttcacaacagattatctgcgccgaaattttccatcgatgaggccaatgaagccgtagaaaagtttttgatacaagcagggcgactcatgtctctgtaaatgtctcctttatgggcgatgtctctgtaagtgtctcctcctttactgaggaagtctctgttatgggcaagaggtttgaaacaacgcaggggctctgcttaatctgctgtctcacaaagggaatcaaactacctgctttcgtatttttaatgtagtaattacccttgttatggtgaattttaagaccatagcgtagacccagcactttattaatgaattttaaaattgtttggggatccgttttactaggctttttaagcttaaactcaaagctgaccgcgcttaaatcatactgaacaaattcatcaacgagttttgtcatcaattgttcattggtcaatatattagggtcctgaacgcatttaaagccgcacttagttaacagcataatggcgtacatatgggattgaaagctataattaaattgcagatcatgatgctctgcatgttgcatggcccattgatggaagtttaattcctgagtttgtaacatagtgagcgattcgtatactgtttttccgcggcttatttggacacggccagtgtagttctgttttgtcataaaactattgtattgttcaacaaatttgggggtaattttatgaccgtgccatgcataaaattcgagtagtttatatttttcatacgcaaataggtcttgctggtctactgtgatgccttcctttaagttttgtttaatttgtaaagctttattggcatcaatggtttcagccgaggcaatgtttacatagtcctggtgcttaatttccattttaatgcttgtatactgtttgactgtctccagcttttcacccgtcagtataaacaccttagcgccggtgtcggcgacctggttaataaatcgtgttataaagtgattttttgatagatgttgtatccgcattgtttcgagccatagatggtagtatggagttttataatataccggcctacctgtttccttactatacgtgaaggaaagctggtgattgtttatggtctgaaagagggtgtcacgtttttgtaacgtgaacatttcaatgtcttcgatggtttctgggtagtaattttgtttcccctgtaagcagattttataacacttactttttaattcacgcacgcggcccaacatttggcaacatgtttctacgtcacacgacatattgttaaaaaagccgtataaaacatcaaatctcttatcttcgtacgaaacacccgccgaaatcgtgggcgtatagataaggatatcaacgagcccccaataatacgatacattattaaaatgggattcccgttcatgagcagtgcttttagaactataaaacccgatttttttttccggaaactttttttggataaatgattgcaacagccgggcctccattaatgaatttgtagggataacaatttttttgtcttctagcaaatcctttaaaaggttatttaaccaagtttctcgtgaagaggtgaaataatacgtgtcatgctgggcccttttatattgattccagtgaaagaagataggaacatccccgcgaaaacgctgtagaatattatacgttcgatttcctaggtttgcgtccaagcatataacataatttgccgtttcgagcatccacatgaaaatggcaaaagagggagcaaagtatttgtgcaggccgctattgaattgattaaaaatcgattctacttcatccaaaataagtaggtccacaggctcggctgtggaggttagccggaaaagtgattccacctgaacgatgactctttcgtagctgtccaaatctccagttacttcactgtataatgtgaaatttggtagccgggactgtatattttttgagaagatctgtcgaaatgtcacaaaccgtatggtttgttgttttgaaatagaattattgccgtagtatttttgcaaatagttgcgcagttggacggttttacctattttcatttgagcctttacaacaagcgtagggactcgttcatattctcgcatactactttcatcatagatgtgtttttgagtatcaggcagttcttcaaagagaatggactcatggacctctatgctctttgtcatcacttggtccacatatgtttccacaaaattagttgtaccggaaaggctgcccatgagaaggctatgtttattgtcatggcgacagtgttgatacactttgtttcccgtgactcttaaaattagggtattatccttatcatgcatacgcttacatatttcgcagtaacttggacttgtacgtttaaacaatactaaatttttatgaacacggaggaagcaatgatttttacatagtgttcctgcaaattttaatacctcttcaagttcactttgttggatagtatcgcaggaactcggtgttgtttcttttacatttgtgaagatacaaggtaaacacgtcgtttcaaagggggttgctatgagggtatcactctttttcgtggttgtactggtctcaaacacctctgcaagctcttcattaaacattttaacacgcatgctaccttttttatgagaccctatgatgcgaaaattttgaatgcttttgttgacctgagggtcaacaaaaggataaacgtgtttgggaagattttctaacactttggatgtaaaggctttggcctcattattgtttaatactgagtatgtataaagtatgatatgaaaggagtatttaagttctcgctttttatttaacccgatagaatctgttagcaaaatttgttcacgcgttagattgatgttataaggtaaagaatatgtctcgtaaaatacatccatgataacgttaattatcatgtcaaggatgtcatagacattgtcatcgtcatagacactgtcatcgtcattgacattgtcattatcatcagagtatgacttatttaccgggaaatcgatgtcaaattttaagcgctgaggcaaaaatccaaataccacttcgtggaaacacttctgctcaaagggctgagccgcctcccactcccaaaagtcatcacgatttgaaaaaaccctaaaaagattattatattcatctcgcaccacgaagtgattctttaaggtttcgagagaatatttatcctctacggcttctccttgggagttacagcgaagaaacttgaatgtttcttgcattttgataattaaaattaaatcaattatgatgcgtcagtggccgctgacgcagccgtcataaataaagcggcggccgtattataacgactagttggccgctataggacgagccatataaaaatgaattcttttaattagagttaagtattgttgattgtataattcattatggttgagccacgcgaacagttttttcaagacctgctttcagcagtggatcaacaaatggacactgtaaaaaatgacataaaagacatcatgaaagaaaaaacatcttttatggtgtcattcgaaaactttatagaacgttacgataccatggaaaaaaatattcaagaccttcagaataagtacgaagaaatggcggccaaccttatgaccgtcatgacggatacaaaaattcagcttggagccattatcgcccaacttgagattctgatgataaatggcactccacttccggcaaaaaaaacaacgattaaggaggctatgcccctaccttcatcaaacacgaacaatgatcaaacgagtcctcccgcctcaggcaaaacaagtgaaacacctaaaaaaaatcccacgaatgcaatgttcttcacgcgtagcgaatgggcatcctcgaaaacttttcgagaaaagtttttaacaccagaaattcaggccatattggatgagcagtttgcaaacaagaccgggatcgaaagattgcatgccgagggtctttacatgtggagaacccaattctctgacgaacagaagaaaatggtcaaagagatgatgaagaagtaatatttttggtaaaaatatttttatcaaaatttttttaccaaataataaaaaatattttttacttttttttcttcataatatacatagaatgcctacaaaagctggcacaaaaagtaccgcaaataaaaaaacaacgaagggctcctccaaatctggttcctccagaggccacaccggcaaaacccatgcttcttcgtccatgcattccgggatgctctataaagatatggtaaatattgctagatctagaggcattccgatttaccagaatggatcgcgtcttactaaaagtgaattggagaaaaaaattaaacggtcaaaatgaatataattaggaagcttaagcctggaacaattagccttgtgctgggacccatgtttgccggcaaaactacgtttcttattcattgcatttacatgctcgaacgtttggaaaaaaaagtagtcttcataaaatctaccaaaaacacccgagacaaaactattaaaacacactccggtatacagctacgacccaagcaatgtaaaatcatagaaagcacacagttatctgacgtgggatctctcaccgatatccatgcagttgtcgtagatgaagcgcatttttttgacgatttaataaaatgccgcacctgggcagaggaagaaaaaattattattcttgcgggactcaatgcttccttcgagcagaaaatgtttccgcccatcgttcgtatttttccttactgcagctgggttaagtatattggccgcacctgtatgaaatgtaaccaacataatgcatgctttaatgtgcgtaagaacgcagacaagacgcttatccttgcgggaggaagtgaactgtacgtaacatgttgtaacaactgtctaaaaaatacatttattaagcagttgcaacctattaaatattaaaaatcttatacaataatggatcattatcttaaaaaattacaagatatttatacgaagctcgagggccatccctttctttttagcccgtcgaaaaccaatgaaaaagagtttattactctgctaaaccaggccttggcctcaacgcagctttaccgcagcatacaacagctgtttttaacgatgtataagctagatcccattgggtttattaactatattaaaacgagtaaacaagagtatttatgcctgttgattaatcctaaactcgttactaagtttttaaaaataacgagctttaaaatttacattaatttcagactgaaaactttttatataagtcctaataagtataataatttttacactgctccctctgaagaaaagactaaccatctcctaaaagaagaaaaaacttgggcaaagattgttgaagaaggaggagaagaatcctaagtcgcttacatttttttttgccattttatagaatgtacacgcatgttgatgttgtcggaatagctgaagcctcagcggccctctacgtgcaaaaagatagggatcgctacttggacgtgctaacaaccatcgaaaactttatttaccaacacaaatgcatcataacaggggaaagcgcccacctactctttttaaaaaaaaatatttatctttacgaattttactccaacaatgtggcggagcacagcaaggctttggcgaccctgctttacaaacttgatccggaatacctcactcgttacacagtactcatcaccaagattcccaaccattggtatgtgattaacgttgatcagcgagaatttgtgcgcctatatgccattccggcagttaaacaacacttaccgattcccattttacccttctattgcaccagcgcactcacccaccaagaattgttttgtttaggacctgaactgcagttaatacaaatatattccaagctctgtaaccccaactttgtcgaggaatggcctacgttgctcgactacgaaaaaagcatgcggatgttatttttagaacagtttccgcgaagattggaaatagcgggcgggaagaaggaggaggaaaagcatgaaagtatcattaaaaaaataatactagaaatggtctctacccgtcagcgaatcgttgttggaggttacatacaaaaaaacctgtacaaccatgtactcaagaatagaaatcgtttacagcttattacgagcttagatatttatgaagaaaaagatatcatccagcaattttgtgattcaaatggactgaagatcaaaatacgtatcaacaatccgctcttgcctacaaatccggaattacggcgtttgactatttattttaatcataataatgatgatgatcagtcatatctaatagtagatatgtacaacacgggaagctatgagctagtgcctacaaatcagataaacacgcttgatggcagttttttaataggaacacccttcgtgcaagcgcgatttttgttggtagagatctgggtgcttatgcttattgcgcagcaaactaaaaaggacaccaaaaaaataatacaattttttataaatcaatatgaaatgcttatgaatagtccttggcccagtatggaggccctttttccctcaagcagtaaaagatatttaggcaactatgtagaccctaacgcgctcataaagtgggcacaactcaaattaaaaagaataccgcctttttatcccggaaagccggatgaagaatcatgttaagccgattgaaaaatcatgttaagccggttgaaatactcttggtgaaagcacggatgtaatattaacattggccgcccgcatttcgtgttgaaatacgatggaggagcgacggctatctaccatgccgatatcggcctggacatcacagttcatgcacttgtagatgggatgactcgcgttatagatggcaggctcgccacagtttctacagatgtaggagatgcagccatccgagtcgtcgtgcgatttttctatgatggtttgcatggcgccctgcgccgtaagcacccaatgctccatttctcccagacgaagacctccgtgcgatcgtttgccgtccaacggctggcctgtgagggcatccgtgggcccatagcttgcaacggcgtatcggtcatccagcacaaatttttgcaggcgctggtgataggtcggtcctatgaagatggccgcatcaaagtactcgccggtctggccgttgaacattttttggcatccattgaagcgtagaccttcttgcgccagtctttctgaaagaagctgcacgttaataggcaggaatgcggtgccgtctgttaccaccccctgtagggcatttgctagaccaaccgtggtttctatcatttgaccgttggtcattcgggagggatgtgagtgggggtttacaatgagatcgggctgcagtccgtcctctgtgaagggcatgtctgaggtgggcagggccagcgccgcaatgcccttgttcccgctgcgagaactcatcttgtcgcctatattgagatttctttcatagcgcaggcgcatgaggccaaagatctcgtcattgggcccatggggacgcatcacagcatccacgacggccggctcatcgaagccgtacatgacagaccggtcgatgtatttgttgagttcgtctttttcgccccgtattttggccacttttcctataatgatgtcgccctttttgaccaccgttcccacgggcacaaacccatctacaagcttttcgtaattagcacccggcttaagatttttggtgattaaagggtcgggcttcccaaacgactctatatcgctttctaattctactttttcttctcggtagaaggtgccggcaaagccgcccctgtcaataaaggactgcgacacgatcacagagtcctcctgattgtagccgccgtagatcatataggccacaatggtattaagcccgttgggtatgacatagttatgtgctatggtctttacaagcggcatttcattgtaaaactggaagaaacggttcatgtctacacgatatggccagctgaagcagtaccaaccccccgtctgcctaccctgatttgtttcataagtaacacgagcaggttgggtacagtttgcgtagggggacactagggcggcaaggcccaaaatagcttggggcacgtccacgtgtgtgaaacgacgtgttacatcatgtttgtgcttgcgtagctcgatgatagagaaggcaacaagacagttttccgcttcttcgggggtaatgaattcacagatgccctgcgctacgagatcttcaagtgtaagtgttccggctaaaatgtcttttgccatttgagacgtaaatcgcgtattttgaatgaaagggattttatgtttttcccagtctttatcgcctttttttctggcctctgcggccttgtaacaggcttgattgtatttttcaatattattatctacaatgagcagggggcgggtcagcctaccgacgtccaaccagaattctacttcgtctaccatgctatcccagtagatggtggtatggggatgcacaatcttgccctcacggcgaaacattctataccgctgagcaagctcaaaggcgttggtgcagcagccaatccattctccgttgataaatacgcgcgctaggccctttcgtacaatgtccttgttggaaacatcggctaactgttgaatggccggatctgatagaaggcgttgttttaacgaaagtacttctccagcggtacagacattggcggtaatggctaactgtttagacatacctactttttcaccagtatcggctgactgggctacgcagatgtatccaggataagatgcgtgcacgcgacgcatcatgtcagccctttctgtttgtttggatgcgttggtggtgttatgagtatttaccgtacgcaacgctgaaatcgtatttaataaattttttctttccaaactttgagtagatactctgtttacaatggggcgctgtcgcaccatgatggttttatttcctgaaatgatagactgttccatactgcgattgagatcggaggcggtattttttgataaagcggcagaaaatgcctcgataatgtttcgctgggttagctcctcaaaagctgtttgtttaagaagttctttgaacccattgatgatgggtgctatcacggatgtattaaaaatagccttaaaggccttagcaagcgagacccctgagccgtgcacccgcttggtgcggtagctatcacggtccgtgggtggaaacacattcataatgacaagaagtattttatgaataagcaggcctaaaaagcgcagttttcgtacacgtgtatctgcggtttggcccatgtgtggcaacaatattttgtctaaaatagtaagttgtctttcattcaagtattgtaccgcattttcatcgcttttgtaagcagatgggtttgagacaaatttggaaaccttctcagataaaaactggataattttttctcggttcagctcgtgttggaccggttgaaatatggggtctaaaacatgaatggatttttccagaatttctatcatgaaggtattcacaggggagttggattccagatcgaataccacttgctcaatgatgctgtcatcgcctgtcattccaaacatgcgaaagatgagataccaaggtatgcgaagttttgagaacttggtgctattgatttcaatggtaatggcaccagtagtcatgtagcgtataataatttgagagctattttcgaaggcacctcctggttgagagataaactcgccgcgaatgatttcattattcccttgttgcatggtatggtaatggatgtgaagcgtgttaaagcggatgttttctaagaggtctacgacccattccccgcctcgggctataaagtagccgccgggttcattagggtcttctcctatttctttttttgcggtttttgataggtgatgagtgtggcagcggttgctgccccgcatgatgggaaatgtggacacctgaaatggaggaatacttgctcgttttacctcctgccgaccattgctgtagtgcgccgttaaaataacctcggcggctagattaacagggcccgaataggaaaggccacacaggcgtgccttattaggtagtaaatttatcttgtttccctgtgaatagtttcgatgttgcgggcgttcaatgttcacatctgtaaagttaaattggatctgaaccgattcccgaagcttatctatttcagtatggtcgcgttggtctttataagtaatatccacgttaaacatttgttttacaatttgcgggattccattgtccataagatcgtcgaagcttttgatgttataccctatcaatcctgtagagtttactgcagcggagataaagctcagcatatcagcctctgtaagctcctcattatccacggtttcaatggggccgtaggttatttgcggccgcaagggttccataattatgaagtactacattaatattcagttattctttaaaataaatctttatttataaatcttatttataatataagaatgccttattcaagagatatcacaaagtttatcacggcaacggaaccagaggtgggtctccccctgttggcgctgcaacactccaagtccgtcataggggttattcttcttgtaataagtttgttatttattttcattggcattattatactatcggtgagtagtggtcataccacagcagcctctatatttatcgtattgagtcttatcctaggtggcggtggtttttttcttatttataaagataattcttaacccacataaaatttgaaaaaatataaagtaagaaaatgtccaattactattattactatggcggggggagatatgattggttaaaaacagtagaacccactaattttttaaaaatcgggttgccttaccaggcacacccattacacctccaacatcaggcaactactactcccccatctatcctagaaaaatttaaacgagcagatattcttcttaatgaggtgaaggccgaaatggacccactcatgttacaaccagaaaccgaaaaaaaattataccagatattgggtagtattgatatgttcaaaggtctgcgaaaaaaagtagaatttacgtacaatgctcaaattgttacgaatgcttggcttaaaatgtatgagctgctaaataccatgaattttaataatacatctcaggcattttgcaattgtgagcttccaggagggtttataagtgcaattaatcattttaattatacaatgatgcattaccctacttttaactgggtagcctcctccctttaccccagttcggaaacagatgccctggaagaccactatggtctttatcagtgcaatccggataactggctgatgcaatctcctttactaaaaaaaaatgtggattataatgacggggacgtaaccattgctagcaatgtaaaaaatctagcgcttagagccacacagaggctgacgcccatccatctatataccgctgacggaggtattaatgtagggcatgactacaataaacaggaagaattaaatcttaagcttcactttggtcaagctcttacaggtttgttgagccttagcaaaggcggaaacatgatactcaaacactataccttaaatcatgcatttactctttctttaatatgcgtattttctcacttttttgaggaactatacattaccaaacctacctcctctcggcccacgaactctgaaacctatattgtgggtaaaaacaggctacgcctatttactcccaaggaagaacaaatacttttgaaacgactagaattttttaatgatacgcccctcgtagacctaagtctttaccaaaatttacttgaaagcatttactttgccgtagaaacaatacatctaaaacagcaaatagagtttctaaacttcggaatgaaatgttaccgacatttttataacaagattaaactacttaacgaatatttagctccgaaaaaaaagatttttcaggataggtggcgtgtgcttaataagctttatgttcttgaaaaaaagcataaacttaagctttgtgcccctcagggatctgttgcctaacttaacagatgcaatcttaacagatgcaatcttaacagacgtaaactaaaaagtgtgttcatacaaggattgtatttatgaatatttattaacatataaggttgtgatggtgcatgtaacactgtataacctatataactacactatgaagcacgacgtataataatttatattgaacacgatgttgactcatttatttgcaaacaaatatttgtttgcaagacgtttgcatgcatttactaatatgttgttgactaatttatttgcaaactagatgtttgcatgtatttatttgaactaatatacactccttgttttatttattatatacacagcatacataagtgtatattatttacacttatgtttataactcgacgtaataacattttacacgctttttttttgcaaaccttaataatattgtatgataaatcaaacaatgtgttatatatgtgatttattattttaggcgccgcaagatgtattccattctcattgcatgcttggtgctattactctgtctagttatatatgtcggtcatcgtgccgatcatgcacgaaaatatttagaaggaatgtggcatggagatccggtttttctaaaacagtcggggctacaatccttttatctctatatacaacctggccacacatgtttttttagcattgtgaataaaaatggtgaaaagctgatggaaaccaaaataccttgtacgataacaaataaaatatatatgttttttaaacctatttttgaatttcatgttgtgatggaagacatacatagatacttaccaaagcagtttaactttctgttagacagcgcagaaggtaaacttattttagaaaacaatcacgttatttatgctgtattgtataaggataacttcgccaccgcactaggaaaaacggttgaaaaatatataacacaaaattaatcatgtattttaagaaaaaatacatcggtcttattgataagaactgtgaaaaaaaaatattagatgattctagtacaataaaaatttgttacatattaattggaatattgattggaactaatatgataactcttatttataatttcatattctgggataattatataaaatgttaccgaaataatgataaaatgttttactgtcctaatgattgggttggatataataatatttgttactattttagtaatggtagtttttctaaaaattatacagctgctagtaatttttgtagacaattaaatggtacacttgctaataatgatactaatttattaaatctaactaaaatatataataatcaatctatgtattgggttaacaatacggtaatattacgtggtgataataaatatagtcaaaaagttaactatacagatttattatttatttgtggtaaataatgataaattaaacaatatgtaatttttatgagtagtaaaaaaatattattattataaaacatatatgtgtataaaatgataataatagttatttttttaatgtgtttaaaaatagttttaaacaatattataatatggagtactttaaatcaaactgtatttttaaataatatttttacaattaatgatacatatggtggtctattttggaatacatattatgataataatcgtagtaattttacttattgtggaatagcaggaaattattgttcatgttgtggtcataacatatcattgtataatacaacaaataattgtagtttaattatttttcctaacaatacagaaatatttaatagaacatatgaattagtatatttggacaaaaaaattaattatacagtaaaactattaaaatctgttgattccccaactattacatataattgtactaattctttaataacatgtaaaaataataatgggacaaatgttaatatatatttaattattaacaatacaattgttaatgatactaatggagatatccttaattattattggaatggtaataataattttacagctacatgtatgattaataatacaattagttcattgaatgaaacagaaaatataaattgtactaatccaatattaaaatatcaaaattatttatccacattattttatatcataatttttattgtgagtggattaataataggtatttttatttcaatcatatctgtattatctatacgaagaaaaagaaaaaaacatgttgaagaaatagaaagtccaccaccctctgaatctaatgaagaagatatttctcacgatgacaccacttccatacatgaaccatctcccagaaaaccattacttcctaagccttacagtcgttatcagtataatacacctatttactacatgcgtccctcaacacaaccactcaacccatttcccctacctaaaccatgcccgccacctaaaccatgtcctccacccaagccatgcccgccacccaaaccatgtcctccacctaaaccgtgttctccacccaaaccgtgtcgtccacctaaaccatgtcctccacctaaaccatgtcctccacctaaaccatgtcctccacctaaaccatgtcctccatccaaaccatgtccttcacctgaatcctattctccacccaaaccactacctagtatcccgttactacccaatatcccgccattatctacacaaaatatttcgcttattcatgtagatagaattatttaataaatatcatgtatttattaaataccacgtatttaatatatactacatgttcattatttaacctttcaagctggtcttcatttaaatttaaaatccactaataaaaaaatgtattttctagtggcggatcatcgagaacatcatgtgattccttttcttaaaaccgattttcatgatatgcaacataatccaatgtttacgcaaaagcaagctctcctggaaatcaagcagctttttaccggagattatctcatctgcaaaagccctaccactattctggcctgtattgaacgaaaaacctacaaagactttgcggcttctttgaaagatggacgttataaaaatcgccaaaaaatgctatcgctgcgagaacaaaccaactgtcaactttatttttttgtagaaggcccggcattccctaaccctcaaaaaaaaattaatcacgttgcctatgcaagcattattactgctatgacgcatcttatggttagagatcatatttttgtcattcaaacgaaaaatgaggcccacagttcccaaaagcttgtgcagcttttttatgccttttctaaggaaatggtgtgcgtcgttcccacctccctcacccccacggatgaagagctatgcatcaagctatggtcttctctttctggtatttcaggcgtgataggtaaaatcttggcaaatacttgttccgtagctcatttggttagtggaaagctttcatcgcagaatattgatcagttaaaaactccctccaatcggccattccccaaaaaagtaaaacgtatgcttataagcattagcaaaggaaataaggagttagaaataaaactgctctcgggggttcccaatatcgggaaaaaattagctgccgaaattttaaaagatcatgcgcttcttttttttctaaatcagcccgtagaatgcttggcaaatatacaaattgttcaaaaaacccgtacgattaagttgggaatgaagcgagccgaagcgattcattattttttaaactggtgtgggtctgcccatgtaaccgatgatagccaaaatatcacagaggcgtcgcggcccgcaacacagcctgctgcaacgcagccattgcacgaagtatcagatgatgcaacatctaatgcatcagatacatcctcacccatagggcatcaaacattatctaaagaaatgttattgaacacagcctgatgttaattattcactacatctaaagaaatgttaacctcgatacttaaaagctattgaacacaactactggggcgctaagttatccaacacatctaaagaaatgtcaacatcctcgatgctaaaagggtcatcgagccggtcaataatgtcttcgccaaaaagtccgggagaactgtaggccgagatgtaatccatggagctatcttccccagagcacacaaagtcctccccaaaaatcataaagttaaatgcaccgggcttacttaacagcttttcgctttgaataatagtgttgagctctgtcagcgcaaattctctcacaatattcacaacccaggaaggctctttaatttcatacagcgttaagaaacttacacataaaaattctatagagtaaagcaaggcgctggcaggatctgttacccgtaggtgtttaaatgtagtgtgatattcattcacgacgttaggcagcactttttccaaatcctccttttcctcatacgacagatgctttacaagcctttcaacatgcataggaggcttgttaaatgtactaacatgccgcaaacagttataattatataagaaaatacgtacggcagagtcgaccgccatgagccttggatcatccattgaggtaggtggtggcggggcaccctggccttccctgatgtctgcgtaggagcgcccctccatggctcctatggcctctatcacagcaggactgatatccaaaatcttggccgtcttgattatttttccgtaatcgaaggtccatggctcctgtggaggcttgggttgtgtttcgctggagggcgtggtcatatctttctttatttgaatagaacggatcgacatcttttccttatcgtactgatctttataattattataatagtcatgaactaattcgggttgagaaagatgatcgtatataatataggtaaaaagtccgcacttgacacattttttatcctggaagtcgtgtaatccccccttggggcagcgtgactcgtagaaggcataaaaggtgttaaattctaagctcgcctttagggctgtttggaccttttttatgtttaattgccccacctcatgttgtagcacgtggcatacagaacagcgtagatcggcaagcgcataatggttgtcaattttttttatgacgtctttgcgtgttacttcaatctcggcgggtttctgcgaactgtcgacggccttgtaaacgtaaatggtccacttatgaggaagccccctttcatcatatagggttgaaatgggaagccttttatactcaaacagccgagtccgttggtcggctcttcctgtgttaggatcaaatatgttataaaatccttgctgagcaagcagggccttttgctcgccataagcattttcgtacgttttgaattctgcaagttcggagttaaaattaggtgcattttgtaaatacttaagaaataattcataggctctaaggtaaatgagagttgaggttttttcctcatcccgtcctccccaccacacccgcaggctttcttcttgaaaatagatgtcattcagacgcgccaactgcgtaaaatcaggccgatatttagaggtataaattttatcataaaattctttttgcgataatagctcggccggggtacgtcctatcacggttttaaactcatattcagcctccttgggagtccgtggcttatgcatagggatgctgccgtcaatacgggccacagtggcagcataatcatacatggggtccagcagaatctctgtcaaaagtaccttggtgtcgtcctgcacgctaagcccttgtagcccattttggtggataatttttttgaaagcctcccgaaaattattagcaatccactgatccgtaatctcagatagctgatttattataccgctatattgctgcatcattttttccaaaagaaaggtcacgtatgcattcaacgagctatccgccttcattccatgaatggtaatcgtaagaaattctttatttttttgcgagctataaatgagattcaaaatataggcatagatgtagatcacagcatacagctgcgttaaaggatcgtaatcctcctcctttttaatattttcgatgctatacacgagcggcaggcagacatttacggctatattggcaaactgtttcacgtccacaagctttccaaagtggataaacgtgcaggccttcatggtttcctgccaaataaaaatacggagcttactattaagatcgccgatgacgcccacatctgccgtacgatcctcttgaataaaatgggccagctcttcgccacaaattttgcaaaagtaggagtaaataagcccctggttgttttctttctccttgtttattcctgaaaatttcattagcttggttcgcatggtgtcgtaggacgcttctgccgcttgaagctgtataagcatgtccacatgggggcaaagcagcttaaacccgcaggctttgcatagattccaattggtggtattgtttttttccttgtagagtacacgaatactttctaatacttttaataactccgcgtattgaagacccgaacgcaactgttttaccagcttgagatgagcacatgcgtttttttcttggagttcccactgttttttaatgtttaggtattctgttgtaataagttctgcctcctgtttcccacaggctttaatgacttcttgaaggatgctgttagggtcatccactttaccctccattgtaagaatttcacgtatagcatccgactgcaccctacctattttttcttccataattttaaaatactgtctcgcctgggtaatgacctccgtgagcttcatgtccacctgctgcagaatcatttgctccttttcacgctgttcagcatgttgtaaaaacttttgttctacagggttccaaagcacctccaaatagcctgctctatataggtcataaagcaagggcatgtatcccgatgtaaaaaccggggacaccgagtacatcgtagacaactcttttaaaaaaaatatcacgcgcttaatgttctcctccggttcaatctcctcggtttcaacgatattagatatatgactgccctgatcctcacggtctagctttcggtgtaccatctcctctgctagccgattaatgagccagctatgcccgccgctccgcaaaaacttataaagttcgatatactggtgcgtaaactggatgatgttttccttggtggttacgacaaccccttccccgttttttttccaggtttcttggtccacgcatttcataaatactcgaataaaattggtcaaattggctcctgaggcgacgtagcccaaggtttcaggcgagaaggagcctatctcagccatacgcataaagcactgcggggaaaaactttttagccgcaacttaagtccatagatttcaatgggggcttctgcgggaacggccaggtgcgtcccattaattaaaaaaatttctttgcgtgtgctagggcgaacacgtaattccttttttttttcgctcacgatggggaccacatcggggtctactagcagttgacgtatgtaggcctccatgggcatggatagatcgggcagctttgactgctcggcgcgaacatggttcacaaaatcttttagagtgaaaagaaagtctattaaacgtatgttttttatatcattagaccctttaagggtagagtagatttcatccactagtgcctcgatttcctcattattgagcgataagatatctgtgccacggtggactatttgcgcgatcgtaattacttcctccattagatagaaactgaatattatatttaaaataaatacaaaatgtcaaatgaaagttttcccgaaacgttggaaaacctactttcgacgttacagaccaaacagcaaaacgcaattcagtcagaggtgattgaatggctgcacagcttttgtgaaacctttcacttaaaaatacactgccataaacagtttattcctagcggggaaaaaaaacgagctaaaatacctgctcaggaaatacagggaaacacgcagccctcccaccatgtgcaccgggttgttctttccagagcacagccggtcaaagcgcaggaatctctgctaacaaccatgtgcaacggactggtgctagatgcaaacacatggacatgtctagccattcctccgcctgcgccctttcaacaggcgacccgtcaagtccaacacttttaccgtaacaatttctacgaagtggttcccatccaggatggcacccttctcacaatctactactgggatgaccccgagcatggcccctcctggtgcctagcaagtacccacggatatgatgtgagtaactactgttggataggcgacaaaaccttcgccgagcttgtatacgaattgctacagcagcactctacctgcgacgtcaccctggaaaaaaataaaacgcggggaacacgtcttttctttaacaacttaaatcccgattactgctatacgattggaattcggcaccataatttacagccgcttatctatgaccctcaaaatatttgggcgattcaatctacaaacctaaaaacgcttaaaacggtatatccagaatactacggctatataggcattccaggaattcagagtcaagttcctgagcttccccagtatgatttaccttatctaatacgatcctataagactgctatgaatcaagccaaaaatgctataaaaaatggcaaaaaagacaaggaatactttaattatggctatttactcatttcgcgagcgcctgccattactaaaagtacttctaatgttttgttaaaatcgcctctgctggtatttttacaaaaaagtgtgtaccagaaaaaacacaatatctctaacagccagcggctagaatttattatactgcaaaactacttgatgcagcattttcgagatcatttcattgctctatttccgcagtacatatcctattatacgaaataccaaaacatgttgaatatgattatccatagtattgcaaccaaagataaagatcatccctttgcaggagccgtggtaaaaaaagtattggaagatattgaaaacgccgaaaacattattgatcatacaaccattcaaaactatgcccatcaaagcaagtacgctatgctttacttgtcaattatttcccatttttaatctaatacggctaaagctgcgggtttttaataaactaacatttaaaaaagtgttttattaaaaattataatacttttattatatatatggaacatccatctacgaactatactcccgaacagcaacacgaaaaattaaaatattacgttttaatacctaaacacctttggtcttatattaaatacgggacgcatgtgcggtactacaccacacaaaatgttttccgagtcggtggctttgtgcttcaaaatccctacgaagccgttataaaaaatgaggtaaaaacagcaataagactgcaaaatagttttaacacaaaagcgaaaggacatgtaacgtgggccgtcccatatgataatattagcaagctatatgccaaaccagatgcaattatgcttaccatacaagaaaatgttgaaaaagctcttcatgctttaaaccaaaacgtactgacgctcgcatcaaaaatacgttaaatataattttgtagaggataaaaagctattttagctaaaaaataattcatatacgtttatgcagaagaagaacggtggctttcaaattcagattgcatccacgtagaccgtagcgttttttttgcttctggtttatatcgtaaaccgtaataaacatcatcatttgtatccgttggatctttttcccactccggataaaaaaccggttttctttttttttggtcgttttttgcagtaagctgtaaattaagggaatatagcttatcgaaaagttgttcctgatccatataaatagcagcatatattaaaaaaaaataaaaaaagacgcttcaacgagtcagtaccactgcttgccaacgatttacgttggttggtgcattatggtgatatagtaatgagtgcctgcacaagtgcttgcacaggtgcttgcacaggtgcttgcacaagtgcttgcacaagtgcttgcacaagtgcctgcacaagtgcttgtacacattactgcatcgccaaagcacctgcaatgcctacttcctcaacagagtacgataactaaatgcttttaagcaccgcttgcgtcgatgtgtccttcggggcaatcgggttcaattggatccaatattattagtcataattatctaatacttattcaattttatcttttttaccttgtaagctttaaacgttgttttaaacaagttaaaacaaataagcttataaatataccatgacaaaattagcccaatggatgtttgagcagtatgtcaaagatttaaacctaaaaaatcgggggtccccctcgttccgcaaatggctcacattgcaaccctcactgctgcgctattcgggtgtgatgcgtgctaacgcctttgacatcctaaaatatggctatcctatgcagcagtcaggttatacggttgctacgcttgaaatccactttaaaaatattaggtcttcctttgccaacatttactggaaccgtgatagcgaggagcctgagtacgtctgctgttgtgccacctatcaatcacacgatggcgaataccggtatcgatttgtttggtaccaacccttcatagaggcttataatgccatagaggcggccctggatcccctggaaaccattatcctgaacctcattgcggcacgagatctagacttcgttgttcacatatttccttataataagggccatgaagactatttggcctccacgcaacttattctcaaaatctttattgcgacgcttttaatggacattttaagaattaaagacaacacgttggacgttcacttaaattccgactatattattgtgatggagcggctttggcctcacataaaggatgccatagaacacttttttgaagcccataaggacttactagggtacttaattgcctttcgcaatggggggaactttgcaggaagtcttagaccctcctgtgggcaaaagattgttcccctaacgattcgagaggtcctacaaatgaatgatattaatttagccgtatggcgggaggtgtttattatgcaggaatgttccgacttagtcatcaatgggatagcgccctgtttccccatttttaacacgtggacgtatttgcaaggcattaaccagattttttttgaaaacacgtctttgcaggagaaatttaaaaaagattttattgcccgagagctttccaaagaaattatcaagggccaaaaaacgttgaatgacaaggagtttaaaaagttaagtctacatcaaatccagtacatggaatcctttttacttatgtcagatgttgccattatgattaccacagagtacgttggctacacccttcaatccctgccgggtattatttcgcgatccagctatttatcccccatcgtgaaaaacattttgatggacgaagactcttttatgtccctactatttgacctatgctatggcgcctacgtgttgcataaaaaagaaaatgtgattcacgcggatttgcacctgaacaatatgacctactaccatttcaacccaaccagttttacagatcgcaacaaaccaggaaaatacaccttaaaggtcaagaatcctgtgattgcctttataaccgggcccaaagtcgaaaccgaaacgtacgtgttcaagcacatagatgggttcggctgcatcattgactttagcagagccattatggggcctaaccatgcaattaagcttgagcggcagtacggcctcgcttttgtaaacaccttttaccgcaatcaaagtgagcatatcttaaaggtattacggtactatttccctgaaatgttaaccaatcgcgaaaacgaaatacagggggtgattttatcaaactttaatttctttttcaatagcattactgccattgatttttacgccattgctagaaacctacgtagtatgctttctttggactatttacacacctctgaggtgaaacgaaacgtagagatttcgcaaacatttttggatacatgtcaatttttggaggaaaaggccgtggaatttttgtttaaaaatcttcatactgtcttatctggcaagccggttgaaaaaacggccggggatgtgcttttacccatcgtatttaaaaaatttttatacccaaatattcctaaaaatatattacggtcttttaccgtaatagatgtatacaattataataatataaagcgttattccggcaaagctatacaaacgtttccaccctgggctcaaaccaaagaaatcttgacgcacgccgagggtcgtacatttgaagatatttttcctagaggagaattagtttttaaaaaggcttacgcagaaaacaaccatttggacaaaattttacagcgtattcgtgagcagcttgctaatgaaaatttgtaaggcttgcagttcttgtatggtcagaacctatgtcgatggaaacattatttttcgctgcagctgcggcgaaagcgttcaaggggatagtcaaaacttgctcgtctctagcaaggtgtaccacaccggggaaatggaagataagtacaagatttttattaaaaatgcaccctttgaccccacgaattgccaaataaaaaaggattgcccgaattgtcatttagactatttgacacaaatctgtattggaagccaaaaaatcattatattggtgtgccgctgtggctatatgagcaacagaggataaaccatatcatcccaccgatttgtgacattcctttaaaaccgtccgcctaaatagttttcacacctttggtggcagactattttataaaaaataatgttggttcatgaagataaagtgtgccaaagaaacttttataaacaaatgattaatgtaggtgctagtcgtgtgtacttaaacagggtattctatagccaagtattttctatagccagtattagtcaagcatttagatgtcagggtatttttatagccagtatttttctataatatgtacaaactattccagtaaacatatgtgtgttctttattgagcagcatcatggcattaacaagtttattaaaccgctctaatgggcattaaatgacaactcggtgcttagcaaaagggcctataccttctagcaattagggccgggaggcattcccagcttttttctataatcagccatacagtacccctgagcctcatacatgggaatgaggtccttccattccttgttgggatcggcgggccagctctcaaatgaggtgtgaatgtaagggtcctgttctttttccttaatgaagcgtttaatctccatttgatgttgtttacttttttgtttgcggcggagcgtgttccgcaccaatacgtaaaaaataccaagaatcacgcataaaagaattattaaaaaaaatatcatcatcgcggggtttaaaaaacgatcccatgcaacaggaatcgttcttaaaaccttgtctggcagggctgtaaacatgaagtctcctcctataatcggggtggggctgtagcctaacagttcaaggtcctgtcgttctagatacttattggcgaactgcccaccctttgcccccgtttttttattaatcaagcagcgctgcattttccaccattctaaatcttcaggagaaagctcaatgccatatatcaattttaacgttattgcatctttttcaatatccttatcaatttggctgagcttttgagctttaagcgggtctagtgtgtacttccatttaaacttagtgtcctgtagtttggctacatgaaatacggaacatttcggtggggcctttgtgacgcccttacactgcggaagtttatcattaggacaggcgcatagatgagactgcgccacagcatcgcgaactacatcgcagacggagtacattttcctcctatgttaaacaataaatttttttcatagctgaaatttgtgggcctatcttttcccttgcccggataataattataagggagtgttgaaacgtctgggagagaattgcttaaaaaatgggtttttgggaggggtaactgcgactgttgtacgtcgttagccagggagattctatatgccgggctaaaggtgcaacgttcctgtgaacaacttagtacgcgcgttgttaatacaaatggactggtattagcaaacctcgtaaactcttccggacttgtttgtttttgtatgatgtttagcagggagtctgccttttcgagaatccaaagcgtcgcattgtagtaaaataaaaatagcgacttatcggcaggcgttgcaaaagcgccgtatagaaaataaagcagtaagtactggggagacaccacaataaggttatcctgaatgatagatatcgctagctctttaaacatggtgctaaaaaaatgtatgtcgttcgtcttgaatatagggggactatagtccatgtagggctcacatatctcagtcaggtgaaggcccatttcttttatgacttcttccgggttgtacgtcgctaacaccagcgcgggataggctttgggcatatccacggtaagtgttatgtttttatcattcttatggtaggagtaagatggttgtggaaattctgttttccactccgggaccttgcaggtaattctcagttcatttaaagtctggtacaggagggcgtatgccgcaaagccgtgtatggccacttgtttaaagggaattgaaaacgttttactttcgtatatcgacttcacaggaacaacgggaatggggtaatatttttctatgaggttataccgctgcaaatcctttttaaacctgctaaaaacatcttcccttggtgggttatcaaaaggaaagcaaaatgctaggtgtagcccggcccgctggtaatcggggtgaatgattttaaggtttttatacgttaatgtgggtatggtgttaaagatattggggggcatatatgaaaggtcagcaacccacacaaagtccgtgcgcacccgcatggtctgcacatggatggcgcgcaccgtgcccacctgcttgaagcccttttcatacaaaatgtcagcaagttcgtaggcatcctcaacgtggttgggggaaaacatatcaaagtcgggtctttctccctcgggataaatcgagctgcctttaagatgcagggcataatcaatggcaatccccccgtacaaaataagctttttctttatgataaattcgcggaccacctccaaagccgcctcaatctccacggcatttgcctcacgcttttgagcaatgagccggtacttagaaacattaaaatcagtctttagtaaagacgacataaatagtgtttaatatatattaaaggtttgaataaaatactaaatagtaaaaatggatgccctattaaaggaaatagaaaagttatcgcagccatccttgcagaaagaaaacaatgatgtatgcgatctctgttttatgcaaatgaaaaaaatttctaactatcagcttttatgcgaagagtgcggtcagctgaaggactggtttgaacctgaatataatgaaaaattcacggtatattctcgtctaaagatcgtgggtgccaatagctcctatcaccagcgcgatttggacaaggccaactcaagtgactatagctctttgcaatttcatcacattttagaggagctcaaatccctaaatgttaagtatatggatgcggggcaaaagccctttcctattcaggtgttaaaggaaactgctcacagttataaccaggtacaacaacatcgggtcatacgcagcattacaaagcttcagatcttagccagtattttacgtagcatttgtttaaaattaaacattgcttgtacggtggcagacgccgcgaggtttactcaacttaataccaaagggatctcaaggggcatggatcttctgcgctccctatttgtagacaataaaattactttaaacgttgatttaaaccctatagacagctttattaatagtacctacagcgccttacaaattaaacaaatccaccaagaactgcaggaggaaaatgtttataatttaaaagaaattgttaagagctttatattatacgcggatgagaagaacatcggcgtcgatcttaacaggaggaccgttgtgattgctacgatgtataatgttttacgccgtgcttactaccccatagaaattgatacggtggtgtatcaatgtaaaatacgaaaaaatacaattacacgtgctcttaaaatgtatgaggattactactcccactttaagtctctttatgagcagtatcatttaaacgcggcaaaaaaattaatttaaactaaacgtttaaactaaacgtttaaactaaaagttaaaactaaacatttcgactaaagtttaaaacctagtctaacagcgggatgcccatttccctggggttccatatttcaacaattttttgaccttcgggtgttaccttgatgcagcgcatgacgagcagtggaattttcctattaaagagttcttgcttagctatatcaataggactgctatatttttttttaagcattgtagacccattaattgccaactgttgcgctctaacggcgaccaaccttgtggcctcaaaggtggttaaaacgttggaggtaatgcgctcgttatcgggtataatgaccaatgtttgcgacgaggcctgcacaaagccctcgcagatggacggagactccacgatctcgtccttgtcctcggactcctcctcactgtcgacgaagttctcctcttccgtttccacatattcctccacgaggtcatccatgataatatcctcgttgtcattatcagccatgttacactgttatcaaatgtactgtttaatacgcaaatggatttactacgttttaattgtatgtcttcatgtgcaggctctagtggatagtaattttctcacaatttttggcaccgttacacttgtgcccacaaaaacccgcgatttttttattttatattacttttggaagtacgagtttaaccagtcgctttcaaaccttatgcgtctatctcgccaaaaaacgctcacagcggtgttggatattacctttaaaaaaataacattaatttttaccacagagggcgtattgcgtatggattctacgaataagccaggcgtgccactcgatatagacccccagttcattgaccttgatagtattttaatggaactggatcattaggacctctcccgcccatttaaatttttagtttctacaataataaaatgcgcgaggaatcatgggaagaccacgataccattcagctcaccgctcagcgcaaatatctcgccgaggtgcaagctctagagacccttttgactcgagagctttcagcctttctcacagagccaggcagcaaaaaaacaaatattattaatagaatcacaggaaaaacctacgcacttcccagcacagagctactaagactctacgagcatctcgagcaatgtcgcaagcaaggcgccctcatgtattttttggaaagacaggggacctactcgggtctcatgttggactatgaccttaaactcaatacaaatgctgttcccccgctggaaccccccgcgctatcacggctttgccatcgaatatttgtacatataaaaaacagcagtgtgctgcctgagggcagccataaaatccacttcttttttacattaaaacctgaagtggtccagggcaaatatgggttccatgtgctcattcctggtctcaagctggcggcttctaccaaaaaaagcatcataggatccctacagcacgatgccaccgtacaaaaaattctacacgaacagggcgttgcaaatcctgagtcctgtctggacccccactccgcctctgttccctcgctcctctacggctcctccaaactaaaccacaagccctaccaactgaaaaccggctttgagttagtctttgatagctctgatcccgactacattcccattcatcaaataaaaaatatagaatcttataatttagtttctgagttgagccttacgaatgaacaaggaagccttgtaaggcctgtctattgcgcggcagacattgccgctgagaaggaggaggagatcccgaccgacgaccactcactctccatattaatgctacatgatcccgaagctcggtatttacataaaattttaaacctgcttcctccagagtattatgtggagtaccccctatggagcaacgtcgtattcgccttggccaatacatccgccaactatcggcccctcgccgaatggttttcacaaaaatgtcctgaaaaatggaatacgggaggaaaagagaaactagaaaaactttggaatgatgcctcgcgccacactgaaaagaaaatcaccaagcggtccattatgtattgggcccacaagcatgctccccagcaatacaaagaaattgtagaacaaggttacttttccattctcgctgaatatgtgtatagctataacggcatgcttgagcactacatgatcgccaaagtcatctatgccatgatgggcaacaagtttgtggtggacgtggactcaaacgggaagtacgtttggttcgaatttgtgctaccgggccagccaatgaatcagggagaaatatggaagtggcgcaaagaggtaaacccggatgagctgcacatctatatttccgaaaacttttcaagggtgatggaccgaatcacggagcacatcaaataccacctcagtcagccccatgaaaccaatattttaaattattataaaaaactactaaaagcctttgaacgctctaaaagtaaaatctttaatgacagttttaaaaagggagttattaggcaagccgagtttttatttcgccaaagaagctttattcaaactctggataccaatccccacctactgggggttggcaacggggttctctccattgagaccatcccggccaagctcattaatcattttcacgagcatcccattcatcagtacacacacatatgttatgtgccctttaatcccgaaaacccctggacaaaactattattgaatgcactccaagacatcatcccagaacttgatgctaggctgtggatcatgttctacctgagcacggccatatttcgcggcctgaaggaggctctgatgcttttgtggcttggaggtggctgcaatggaaaaacttttctaatgcgacttgtggccatggtattgggcgatcactatgcctccaagctcaacatcagccttcttacaagctgcagagaaaccgcggaaaaacccaacagtgccttcatgcggcttaaggggcggggatatgggtactttgaggaaaccaacaaaagcgaggttctaaatacgtcgcggctgaaggaaatggtaaatccgggcgatgtcaccgctcgagagcttaatcaaaaacaggaaagctttcagatgacggccaccatggtcgccgcgtccaactataacttcatcattgacacgacggaccacggcacatggagaagactgcggcattatcgttcaaaggtgaaattctgccataaccccgaccccggtaacccctacgagaaaaaggaagatcctcgctttattcacgagtacatcatggatccagactgccaaaacgcattcttcagcatactcgtctatttttgggagaagctacagaaggaatacaacgggcagattaaaaaagtgttttgtcccaccattgagagcgaaacggaggcgtacagaaagtcacaagatacgctacataggtttatcacagaaagagtcgtggagtcgccctcggcagaaactgtgtacaacctatccgaggtcgtgacggcctacgcggaatggtacaacgccaacattaacgtaaagcgccatattgcccttgagctatcccaggagttagaaaactctgtgctagaaaaataccttcagtggtctcccaacaaaacgcgaattctaaagggttgccgtattttgcataaatttgaaacgctgcagcccggcgaatcctacattggggtgtccacggccggcacactcctaaacacacccatatgcgagccaaaaaataaatggtgggaatggtctcctaatctctctgcccctcctgagaaagaagcgtctgcaccaactccttaggaaatatccttagaagcatgtctttcggcagagccattaccggtagcaaaaaagcaacattgagtatattatatgccttagcctgctcataagcgtccttttttttcatggtattttatgtttttatatatttttaattattttttaaatacgatgaacagttcgtgctccgaaggctgtttactaaaaatcggtgtgaatccgcattctttaaatatggtttcccattcggggatggtatggaaatccatgtctctacgaatagtatggtgcccaagcgcgtcctgcaggctgtgaagccagaaggcttcctgacctttatgaaggtcatacacgataagaaaaccatcaggtttcaacagatggtaaagcttgttaaaatcgtttatcgtaagatgatgcgccgccataggtaaccctatgagctccacagagttttcatgctggacatcgtccatatcggtataaaacgtttcacaataaatgagacgcttaaacgagtattgatgacaaacatttatttccaagtaggtttgcaccacgtttttaggtatatcgggaatcatgttgattaaggttgtttcgggaaacttaatcatctgactaggcttcattttcaactctttaaaggatttcccggagaagtgaaaatgggtctttacgtatttatgtaaaaatacctgaatggggagagggggctcctcctcttcgtcctcgacgcctcccaaaatatttggaatttcctgacgtggcgaaagaaagtttatgtccacgtttacgaatccatcgaggacggacacaaagcttggctctaatctccattccatatactgtttagaaacgggagatagcataatcctaggcgttacaatgcacgaaggatttttaatcaccgcatcgtggtaagaaaagtgtattccatttcttccagtataaagaagcctatgtttgtcgtagcagaaacaattaaggcggtatgcctcatacatacactgtttcaaagtacaaacacgttttaaaaaggtttctgcattggcggaggccaagcggttttgccattggtggaaggggttcaatcctacaatggccagctcgtttaaaatatcttcgcggcgcgccaaaatctgcaccatagaagaatattttagcatttttttttcgcaccattcgcgaagatgtttagctacattattaaccttattattgataaagtatacgatggcatgttggaagccttcaaaaataaagagcccctccaaaagatcatctgccaatagaagatggatgttggtgtaagcattgtcaatattttgtagaaacggcggaatgcctgccaaaaccgcttcagcaagcatagctccgttccgttgtttactgtccaatagattcgtaagttttttgtccgcaacagacacgacggctaggatggttgcaatgtcagaaatggcggcttgccagaaataacccgaaaagcacatgcgcgcttcttctatagataaaaacgaaaagcgagaggcaatgtctccgagctgcgtgagttgaagacctttttctcctctggttaaaaggcctgccacaatggctcgctcaatggctgatgccagcgcatccgtggggggaggatccagcatatcaatctcctctgccttaaacacgccttccttattttttttaatcgtttctacgacaatgctaagaaaaatggccccagggccttccgtaatgatttcaggatactgctgcgctggtatttgctcaaagacgtgttttgtgtaaagcgggtaaaagtgcccaggaaatactctccctacgcgtccctttctttgctcgatacggctttgagccgcggggcgcgtaatgagccctcccgcccattcagggtagtaggtttcaatgcttctgttccacccgggatctatgacgtacttcagcgtttcaatggtaaggcccgtttccgcaacaaccgtggaaacaatgacccttcttaaaggtttttccactttagcggttaagggatttttcacccacagattcttaatttccgctttcaggccaaggtaggcctcattttcctgcgcaatcgcctcactatcgatcggcaaaatcaacattaacggcagcttttctttggcaaggtccatatttgcattattcagcaacatcgaaaggaagcgtatttcagccataccgggcatgaaaattaaaatatctgcttccgtgggacgatcatgaatgttttctttatgaatagtgagagccgtttcgcaggcggtcttaatgtagttgttggtgttatacagcggccagtgggtttccacaccgtactgtcgtccttccaccaaaataatgttttcttttccgataccaaaataggttgagtatttatgggtatcaatggtggcggaggttaaaattacaaagggaatacgcagcgcccctatgcttcctctttgcaacatgcgctgaagcatacttttaatatacatgagcataaggtcgatgcctagggctcgctcatgggcctcatctataatcataaaggcatagcgggaagctatctcatcatccgtcattgtatgtagctgcgccaacagaacccccgcggttgcatagataaggccccgattgggtttttccgtcagaggcttcgtctggtagcccactgtttggcctaatatcatgtcggggtagtgggttgaggcgccgatgtctttggcgagggtcactgcggttaggactcttggctgggtacaaataaccgagcgtcccaagtatttttggaaagaatgcgtgttttcatttctcagaattctgaacacgtgtacgggcagggccgtggattttccggatccagtgcgtgactttataatgagcacccggtctgcgagggaggttggaatggcccctccaaactccgggagacgttgttttatccacgtgatgatgtaatgaataggaacatcattcttgtgctcagcgggcacgttatagagatgaccaggctccaataaagtcggttttcccatattctattgttttaaggattgattgttcataaatatttttatactctgaccaagaaattatttttttattaagccggttatttacgttgttatggaacgcgaaggtccagtactgaaagtcctccgagttgtttaatgtcaagggattttttgtaagatacgaaaaggcgtggtgctgacacctggtgcatggcagagactcgataaagttcagtatccattggatggcttcatatttttctttccagctaggagcgtctgaaaaaaagatagcatatagatgcaaggatcgccagtatttaggtccccaatgcaacatttataaccttttgaaaaatctcattccatatagaggtaaatattttttttccatggagaatttttttgcactcttgaagggattgcgccacatcgtcaaatgttttttgttttccatgtattttggcgtaattccagccagtatctgtgtcatggtccttaatgtcatccgctaactgaaaggcatgtccaaaacagtgggcagccctttcaatcatcccaatgtcttcaatcgttcctgtgcccaaaacccagcccataataaacgcgatcttaaaaaagggaatggttttttctggagtgtctactaactgaccggaacccgcgctgtttagagagtggcttacaaaggtacacagcagcgctcccagttggttgggatccggaaaccttggacagtgttccttaatccagtcgatttgccggcaaatgttttgaaatccttgcatggttagcgccagagcgctcatctgcgccttggctacgccaaagcgggcccacactgtatctttatttcgccgcttcacatcgttgtcaaaggagggcatatcgtcgataatcaaagaagctacgtgaaagtactccactgctagggcagcctctgccggataaataggcgccccaaaggaatgttgcaactgacaggcccgaacaatttccatcaggataatgggacggatatacttcccacctcttagagcgtaagagcaaggctctgttagttgtcccttaaagtccccatcttcaatagcattgtttaagatggtctcaaactctttactaaaggttttataatttttaggattcagtggatgtattccatgaaaaagcgcgacactacgcggagctgtgattctaaaatacttaggtttgcgcgtataggatattaaaataataataagaactacaatgatggagatatagatgagatgcaacatgctgagttgtctccccgcagggaatggtccttttccgcactggttaacggtactgaggaggcgttgaaatctttaggaaaggtgctgtctagtttggaatctccaattcctcccgtatatttaggtatataattattgtgcctagaaattgtttgctttgaggtatcaaaatattcagcctgaccgctatttcttttagaataattcggtatagggcttgagtagttggcaatactcttaaaccggggcaccaaggtaacaatattttccatataatgggtttgatacgctttgtttaaaaatgggcttaccggctttatgcttgttagttgtgcattgagtaccggtatgtcttctaggatttgtggctttatagaatgattagcaaacacagaatgtagtatattagctacttgtagcatgtgtctatttgcggaaaattcctggtattctctgccgtgttgcgaatctttgggcggaaggggaccaagcatcggcacgcccgtgtaggtactggtggattttatgagttcctgctctatgttcggtttgacatgtggatttcctaaaggaatacctctacctgcaatccctttttctaccgacgcaggtagattgtgcgctaaacacaaaatattgtacacgtctttgtgcggaatatatccgttatagtgctggcccggcatctgatcgccaaggtgctgctcatgcttaatggtaccctttgttttgagtttaggaatatcctcgtacgaaaaaaattttgtgtgctcgctgaacctcgtagacggaaccgaactattttttgggttttttaaggaaggcaatgaggaaggctgggtcagacaatttttctgtgtgccctttaagctagccacctgcggaaatgtttttttttccgtacgaacaacattgcgcctaattaggttttccgtatgggttgaaaaagcaggacgatgatttttaaaatgattaaaaagtttattttttggaatggagctgtacggctccagatcttgcgcatcgccgtaaccaatgtttttgtgctgagggttcagcataaaagaaaagttacgtagatcactgagttgcaatcccttttcagccttttcaggactattagtgtattcattgtatacaggcgcagctccatttttgttgccgcagtaccgggaatttagtatattatcagaataccggttatgacgcggcaaatcgctttcccaaagaggtggatctgacctataatcggctaacagctttgaagcataatcatgatacattgtatataaaagttaattattatattgagaaggcataattacctcttgtaggggtacaagaggctttgaatcagccgaactggccggctttgaatcggccggctttggaccggccggctttggaccggcaggtatctttttaggttgatcttcttctagctcattagacacggatgggggagaaataggaggaataatttcatctccgcccttatatttgtcgtggatagaagaaacaattacatccatgtttgatttattataaatgtcgtttaactggtgatttaaaacataataatgcaaaaataatagggctacaatgcatatatatacgtaaatagccgtcttcgtttttcgttttttatccaccggcggattacaaattgcaaaaaatacaactaataccaccgctgtaatgattaaggccacaatgaaaggattttgaaaggatgttttgaatggttcgcacgtataaattttttctcctaaattattgatacccgcaataaaatctacattcattttatatatttataaattatgaaaaatttaaagttacatctccgccggaccaatcattgctaaaatttgaagattcttcaaaaaggcccgactggttgaatgtcttctgctcaggtttccaaaaattttccaagaatggattttgaacaataggctcatcttgattttcttcttcaaggatattttctttggtatcaagaacagcttctttaaactcaggtgtatcttgattaaactcaggtttatcctgatcaatcgcaagaatattttcttcttcaggtatatcctgtttaatcgcaagaatattttcttcctcaggtttatcctgatcaatcgcaagaatattttcttcctcaggtttatcctgaccaaactcaacaatatctttctcgctaaatccgtttttagtgtgaagttcttggttttgaagagaattatcaaaatctagtttagttgttgttctagatcgtggcacaggatagttatctggtggtttacttactatagtcctcgaatgtggcacgggataattgtttggtgacttgctggttagctcttggcttgttaatagttcttgttttctcaataattccatctctactacttctttttgatccgccggtgtctctttttggtattcttcattagaaaaatgttcagagggtaatgtttcaataaactttgtgagtggatagctgctctttgatgtagaagagcgttgaatttgctgataaaggagttgaacaagtcgccggtattcactctgtcttttttcatattttttacgtagcgtggagagatccgctaagagcgactttttttcagatgttaattcttcaatttgatgaagaaggctgcgattgtatgaactaagtcttgcatacgtttcttctaattctgtctccggctccacataggcctgttttcgcaaaaatttgttgtatagttccattctttttttgagcagaaaggtaagactataatcttgcatttctttcgtaactttatggtagttttctttccggtttttgataataaagggcagcattttttctgttgtgataaaggtgcccaaattgctaatgtagtcgcacagtagcaattccaagatagattctttcttttcaaggcttatagattggctgtattctttaggtatgaaagaatcaacaatcgttgttatgaagtttgaaaagtttaatgttttgctgttaatttgggtaatgttacaaaaatatttgtaaaaactatctagcattttttcataaagttttttattttgtttaacccctaaaatatagccctttacttgatactgatattccgtaacaatggaatgttttttgtatagtgcatttttgtataaaaagttataaaaaatgttgataaaatacgcaccaagggtttcaaaaatacttataacgtgggattcttcctgatccattatatcatatgtaatattattttaataaaaaattactgactaataacatgcaaaaaaaatatgtttaaacttattttaagctagcacttatttaaaagcttgttttaaacacgttttaaattgtatgttaatacagttaaaaattaagccgaaatttgctccaataaggattacttttatcaatgaccacctctttactataaacggctttacataattttaataaagctttggagccaaagctgaaggcagtgggaagcggcactgtactatggtaaaaatgttgccgattttcatcctcgcggatgtacacaagtttcctatatcctttaaacacaatatggctaatttcttccacatactccttatcctgtttggaatagcggttgctttgtcgggaaaaattcgacatacaaatagaggcatttgtaaaaatggaaacaaatgcgtttttacgaagattggcgggtaaatcggtgtcatcttggcagcaaataatcatcgaaataaaacagtgacgattttggtaaaaaaactttttaaaaatttcttttgtaaataatgggtgcagttcggccgcgcagtcgtctaatattaaaagtaaacgaggattaagattgatatagtttaacgtaaacttttcatcctctgtaaggcataagtttttatacatatgaatgttctgtataataattttttttaaaagttgctgataaagcgatgtaatcttttcttcttttttttggtccgtttgttcagcctttaagcactccacttttgcaatatttttgttttccttttgctgtatatcgatcggaagtttatgatacaatgtttttagcatatcgatgttgtttactcgactgtagatggaggacatcatagtttgccgctgccagatggcctccaaaaagcgttcagcgcccttgttgtcattttttttttgcttatcggcgagccacaagcggtagtgaattagagttggatgtacaaaaccctcatatgaacgatttgagggttccgagggggcaaccactaaaatttgttcaatatggggttgcaggattttcataatatgtttaacgtacacggttttgcctgtttttgaggggccatatagcacagttgttttatctataaaatgatgtgctttgaactgtagttcaggaatcagcttccctgaatgggtcgttagggccatctctatattattacaattctgcttttgtatataaaatttctttttcgagtttattattattgttgacccacatatctacccgtatcgtatcatcaggcacattgagcatttcaagcgcattatttaactgtttttttgtttttatcagctcgctttcttcatcgggggttaaattttctttactaagcagttgcttaattttttcttcgcagtcgtctataaaatcatactctcgagcttttttgatatttccagatgctttttctaggttttttagctccttaaaggaaagcagtcccttaatcccgctatccgtgtgaaaggttgaattatagatggagagccccggagcatccgggccggtttcttgtatattttttgcttttttgtggtaaatggtatttcgtagaatctcttttcctatctttaggtcttcctcatgacggtccaaaatccgttttattatttcattattttgattaaaataattgtagcgctctctgttggccttaaagcttcccaggagtgtccagttgcctaattgaatggatgaaacctctgagaaaatctggtctttatatttataataaaattcatcaaccttttgttggttgctgctatccaccacatcataaataatgaaggcaaactctaggtcgggcttttctgggtagatgctttccgtagcggcccgcaactcttcgtaattatcctcaatgtaataattccacttataaaaagtatcctgaggtggaatatgctgcgaaagatatctagtaatttttgtgttaaagagaatgggtttaaacgccctcggattttcaagcatatgtttaatgctttgatgaagttctatattttgtaatatgtgggctgctgccctatagccctgtggggtttgggtaattgcatcaatatcggcctgaagctcattaggcacatttaatgttttttgcatgatgtgtaaagggatgcgctcaggatctgctaaatcggtgtattctgtgcttgcacaggtgtttgcacaggtgcttgcacaggtgtctacattggtatctgcacacatgcttgcacaagtgtttacattggtatctgcacaagtgcttgcacaggtgtctacattggtatctgcacaagtgcttgcacaggtgtctacattggtatctgcacaagtgcttgcacaggtgtctacattggtatctgcacaagtgcttgcacaggtgtctacattggtatctgcacaagtgcttgcacctgtgcttgcacaagtgcttgcacaagtgcttgcacaagtgcttgcacaggtgtctacattggtatctgcacaagtgcttgcacaagtatacgcactttgagcatgaagattaggatcaaacacaaaatgttctcgtaaaaagctatcgatcgttgttttagcttccttgcttttctgcgtctgggttttgcagctatccgctatagataaaattgtatttactaccgattcagagggaacatcattagtttcctgtttcaaagtatcaactaacgttattagctcactgagaagagttttggtcgtgtgggtaggttttgaataggaaggcatccattcctgcagagctttgaagacatatccaataaagctagtcattataagacgtcgaatatactgctcccgcaaatttgtaaaagagcaaaaggccaccctgctatcatttttgaactgtttgtaagggttcgtcctttgataaagctgtttaagcgtttcttcggatatttcagtagagggatcctccaatacgtttttgagaagctcatcaatattaaattctgccatatcttagagtttattatatacatattaaagctttaatataaggggggtataacaatggacgaaatcatcaataaataccaagctgttgaaaaactttttaaggaaattcagcaaggattggccgcgtatgatcaatacaagaccttaattagtgaaatgatgcactataataatcatatcaagcaggagtattttaactttttaatgattatttcaccttatcttattagggcgcatagcggagaaacgctgcgaaacaaagtaaataatgaaattaaacgtcttattttggttgaaaatatcaataccaaaatatctaaaacgctggtaagtgttaattttttactacagaaaaaactttcaacggacggggtgaaaacgaaaaacatgtggtgcaccaataatcccatgctgcaggtaaaaacagcccacaacctttttaagcaactatgcgacacacagtccaaaactcaatgggtacaaaccttaaaatacaaggaatgcaagtattgtcataccgacatggtgtttaacaccacgcagtttgggctgcaatgtcctaactgcggttgtattcaagaattgatgggaaccatttttgatgaaacacatttttacaaccatgatgggcagaaagcaaagtcaggtatctttaaccctaaccgtcactatcggttttggatagaacatattcttggtagaaatccagaacaagagttggggaccaaacaagatccctgcggaaccaaggtgttgcaacaactaaaaaaaattattaagcgcgataataaatgcatcgcgcttttgacggtcgaaaatattcgaaaaatgttaaaagagataaaccgcacagacttaaataattgtgtttctcttatattgcgtaaacttaccggagtagggccgcctcaaatatcagagtcgattttactacgaggcgaatacatatttacagaggcaattaagatacgggaaaaagtgtgtaaaaaagggcgtattaataggaattattatccgtattatatatataaaatttttgacgccattttgcctccaaatgataccacgaatcgacgcattttacaatatatacatttgcaaggaaatgatacgctagctaataatgatagtgagtgggaatctatctgtatggagctccctgaaataaaatggaagcccacagatcgaacccattgtgttcattttttttaaagatgaagattttttagatgattttttttagttttttaaaaagacgaaaaaattttttaaaagacgaatattcttaatccccgcaaattactttttttttaggtactgtaacgcagcacagctgaaccgttctgaagaagaagaaagttaatagcagatgccgataccacaagatcagccgtagtgatagaccccacgtaatccgtgtcccaactaatataaaattctcttgctctggatacgttaatatgaccactgggttggtattcctcccgtggcttcaaagcaaaggtaatcatcatcgcacccggatcatcgggggttttaattgcattgcctccgtagtggaagggtatgtaagagctgcagaactttgatggaaacttatcgataagattgataccatgagcagttacggaaatgtttttaataataggtaatgtgatcggatacgtaacggggctaatatccgatatagatgaacatgcgtctggaagagctgtatctctatcctgaaagcttatctctgcgtggtgagtaggctgcataatggcgttaacaacatgtccgaacttgtgccaatctcggtgttgatgaggattttgatcggagatgttccaggtaggttttaatcctataaacatatattcaatgggccatttaagagcagacattagtttttcatcgtggtggttattgttggtgtgggtcacctgcgttttatggacacgtatcagggaaaatcgaacgcgttttacaaaaaggttgtgtatttcaggggttacaaacaggttattgatgtaaagttcattattcgtgagcgagatttcattaatgactcctgggataaaccatggtttaaagcgtatattgcgtctactggggcgtccaggtataaaacgcgactggcgtataaaaagtccaggaaattcattcaccaaatccttttgcgatgcaagctttatggtgataaagcgctcgccgaagggaatggataccgagggaatagcaaggttcacgttctcgttaaaccaaaagcgcagcttaatccagagcgcaagagggggctgatagtatttaggggtttgaggtccattacagctgtaatgaacattacgtcttatgtccagatacgttgcgtccgtaataggagtaatatcttgtttacctgctgtttggatattgtgagagttctcgggaaaatgttgtgaaaggaatttcgggttggtatggctgcacgttcgctgcgtatcattttcatcggtaagaataggtttgctttggtgcggcttgtgcaaatcatgaatgttgcataggagagggccactagttccctccaccgatacctcctggccgaccaagtgcttatatccagtcattttatcccctgggatgcaaaatttgcgcacaagcgttgtgacatccgaactatattcgtccagggaatttccatttacatcgaatcttacgttttcataaagtcgttctccggggtattcgcagtagtaaaccaagtttcggtacgcattctttgtgccgggtacaataggtcttccaaagggatctacaagcgtgtaaacggcgccctctaaaggtgtttggttgtcccagtcatatccgttgcgaggaaacgtttgaagctgaccatgggcccccatctgggccgtgccctgaatcggagcatcctgccaggacgaatgacatgcacccaatatatggtggcccaccatatcatggaaaaagtctccgtactggggaataccaaaggtaagcttgtttcccaaggtgggggtacccgtatgcgggcgtactttattgtattcaaaccctactggaacataaggcttaaaatgcgcattaaaatgaaccaaatgtgtttcttcgatttgactcaaagtgggttcggggtcgggtttcccataacttttgttcacatttttaatgttagaaatcctgctattaagcaagtcttgggccaatataatcttgtcggccttcccatcgttagcaataagacaaaaagctcctcctgatgccatatataatgttataaaaataatttattgtttttattaaatatggcggtttatgcgaaggatcttgataataacaaagagttaaaccaaaaattaattaacgatcagcttaaaattattgacacgctcttgctagcagaaaaaaaaaactttttggtgtatgaattgcctgccccttttgacttttcctccggcgaccctttggccagtcagcgcgacatatactatgccatcataaaaagcctcgaggagcgcgggtttactgtcaaaatatgtatgaaaggggatcgtgccctccttttcatcacttggaaaaaaatacaatccattgagataaacaaaaaagaagaatatctgcgcatgcacttcatacaagacgaagagaaagcattttattgtaaatttttagagtctagatgagcttttacgcaatgttgtacagtgttgtatatatgtcttgtaagcatttgttgtagagtaataagtaaaagataaataaaaatgactattaaaataaagcccaaaccattaaaaatatttttatctgttagatttaatttaataaatggctcatggaatgtgtggtgcgccgctgcatgaggtgcggccacggccgcatgggatgtggtcgcataagatgtagctacatgggatgtggcatttgcttgcatgtaaggatcgtgatgtgttgggtcttcatcccagcaataatcgccatctttatctagctgaattgtataccccattatatatcacttattatttttttttaatgtttcatgaatttcattataggcggtgaaagggtcctcaggccccttctgtaaaagattatagagatcttcggacgctttatgtttcgtgcgaattaaggcgggatataacaaaagagagggccccagttccaaacaaattttacttagcgggctcatattttgcaccaagtttcccactacttgcgatgtttcataacgcattttaaagagctttatcataaaagtgttgtgcaggccggtgtagtctggcctatagttaaggaaggggatttctctggtaccgtcaaacacgatctcaagccctctagcaagtccgatcaaaatttcttcagcaatggatgagtatctaattcctacattacgaagcgtaagcatttctataacatcatctatttcctgcatagaggaatctattgtaggaattttaatatcatctgtgctgatttgttcattcccaagataggtaagcagcatattaattttttctagctttaccagcttagtcttacgctcataatcatggtcttttttataaaaagagttgggatcaccgttggaccgtagatgattaataaggcggtctacttgctttgtgctaggtttaatacttttttcactatactcgctttcagcatagtggtttttacgatctcttttagaaaaagctgttttttgagatgcctcagactctgcatatttttttctatgcgtagaaagagaataaccgcggtcattacgtgaactactgttgcatgcaaggcctcggcgcgtcttaccgctgcgcacactgtcatcgcgcacactgccgctgcgtatactgccattgcgtatactgccactgcgtatgctgccactgcgtatactgccgctacatacactatcactacatatgctgtcagtacatacgctatcgcggcgtatgccgccgtgtaccttatcgcggcccctacccgagggttttttagatataacactatgcggggagtcaagcgaaaattcaggatcattaaagttaatgcccaatgactttgccaatccattaagctcttcatcgaaatgatcggtaggaaaactttgttgtttgcccatgacctgtttttcaagttcttccaaattggcttgctcatttatatggagattattcataagcgttgtaatcccagcaagatttgctccttctaaaaatgtggtgtcctccatcggatatactatactatttaaaagcttttaaataaaaatgtgtttggaagaaatgctctcttcaagcgtgtgtagctcagatataaacgcctcctcagaaagctttccaccatactcctttctcatcgtataggagggcgccggcttaatgtaggaaatccactgggaggtaaaaaaccggtacaacatatttagcagctcgcgggcctcccaccttttgggctccgtatagtgcacatcaacataagaggcggcgcatgaaaagctgcaaaagttgccgagaacgcccatctcaatctctcctcgctcattttcacgcatataggtgggcacgaatttcggaacagtcttgaaatagagatgacacgtccagcatttaaagctagaatgggtaacccatttggaaacagtagtgaatacggagggtagctttttttcgacctcggcttcatcgtcattcgtatttaacgtatcggtggcagtttttttggtttgcaagcattcttcaatggtaatcccggataagtataaaatattaggacaattagtttccataattttgatagttatttttatacaacatggatttaattaaagataaatggaggacgaaacggaactgtgttttcggtcaaacaaggtgacgaggcttgaaatgtttgtctgcacgtacgggggaaaaattaccagccttgcatgttcgcatatggagttaattaaaatgttgcaaatcgccgagccggtgaaggcattgaactgcaactttggccaccagtgcctaccgggctacgaatctttaataaagactccgaaaaaaactaaaaacatgttgcgccgtccgcgcaaaacagaaggcgatgggacctgcttcaatagtgccattgaagcttccattttgtttaaggacaagatgtataaattaaaatgttttcctagtaccggggaaattcaggtcccgggcgtcatttttccggattttgaagacggaaaaaacattatacagcagtgggtagacttcttgcaacatcaacccattgaaaaaaaaatccagattattgaatttaaaacgatcatgattaattttaagtttcaaataaacccagtgtctccccgcgtcatcattcatttaaaaaaatttgcagctttgttggaacacatccctactccatatcccatacgtgaaataaagcctccattagaagactcaaaagtatccgcaaaatttatggtcagtccgggaaaaaaagtacgcattaatgtttttcttaaaggtaagataaatattttaggctgcaacacaaaggaatccgcggagatcatttatacgtttttgaaagatcttatcagcgtacattggcaagaaattttgtgcgtgttaccggtacccgattaaagaatgttttcattaataaggtaatcgacgatgctaaaaagaataacaagaaaaataccttgaagaactataccaaagtaggtaggttttctgcatgtcacggcatggttaaaattgctaataatgtagtccacaaaggcattgctcaatacgactaaaaatagtaaaaaaaggataagtgctctttttatatccatatactttaaaacttattttttacactaataatttcctgcggccgcaatataaactgtaggtcatctataacgcccagacctgttaaaagtagagtactatgttttaagggatttaaaatatccgccgcgagaatgtgaatataattttcaaagtggtttacaggaatgcgtaagcgtttttttttgcactgcggttggtttagggtcgaatactggcaggaggtatatatattaataagaccgcggtcgatggtttcaatatcttcatagaattcaatgcgcggcgtcaaaagttttttaagatgttgacataactcatcatacgtgtaggactggaggggggaaagaagggtgtaatcaaagttaaaaatgtttttttgaagaacctttaaagcatgttccgcgtccgtggtttccaaaatatgttttatggtatgaatgtcatttaaatctacaaagtctgacagctttgtgtagaactcggtgacggaggttattttctggaaatcggttttttgaaaaagattttcaatgtgtttgcgggttgagttgctttgcagtccatacaagacatcaaaaaattcaatcagcaaaaacttatacaaatggttaatataaaaagctttgttggccttattctgctgaggatatggttcctccaggggatatagaatggcttggtctatatccctaggatcaatagtcaatgttgcgatgggaagcttttccagcgtagcgggaagagtttgggttggagcgtagtaaaagtatagcccggtttttccctctgaaagaaagcccacaaattctttttttatattttgcagcaccgctgagggtacgatttcgtactgtttatactgtttgttgaaaagggtaataaatttccaggtttcttcaaagcttgcaatctgggtgggccgcagatcaaagtcgatgggaatgtcgtcatgaatgtaggatgatagtcttataggaaaataaatagggcgatcggtgtctgaatcgataagtaaagcataacaaaagttatgcctgttgataagttttttaccaaccgtgtagccgggaatgtttttcacgtcatggatatcccaccagttatccttacacataaactcgctcatagactggacgacctccatcacagggtcatcttcggtaaaaatatactgggcctcactgtttttcagaaatcttttttgctgggtgatggccattgggtagatcccttcgtccgtgtcaaagataatggctatcttcttcgatgggctaagaattttttgtattgtgctgggggacacctcaaactcgatgtcgccctgtttatctttaaaaaagacacagtgaaggtcgtagcatatggcaacaaggtccagaaagatgtcctgccatgtggtgtcccactgaagcagttggtttttttgttcaacaaaggtttgtaagataaggtttgccagctccgcgccgctggaaaacatgttgccggccccattccccaaaatatagtactgcggcgtgttggccgcctttgcaatttcaatggcaagggccttgggagcaagatccaaaattcgagcaagggaataaaaaagcccggcattgctaattccaagcatggtttgctccacccccacaatgcaaaaaatgtcgggctcttttatcgtatttaaaaacagttcatctgctatttggtggggtagaaaggcaatccggttcaccggtattttttttccataggacaaggtatgacgcgatgtttgtgtattaagatcctccaggtcttgttctacaaacgtgtgcttggtgaggcaggtattgttaatatagaaccgctttgtgcccagcagggccttcgtcttttggcagcacggcagacagtaatttagggggtggcggccttctagtaggcttagatgagggtagtcaggatgcgggcagctatagtaggcaggtaccccctccgtgaaattccaatactttactagctccttgcgcttggctggcggcatggacttcacctcggcctctgagtaaatgacgggtggccgtgggtgctggcataggacggagtaaaccgttgcctgcgtgtcgtacttgcgcaggtcatacaggtcggggtcctgttcttgaagcgcacgtagctgagaggctccctttccttgttgtttatcgtgcagttgagagagtttattaaccaaaattttgtcaggcccggtgatcaagttatctaaaaacacaaataggtaaacccaaagatagttaaactcttcctgggtaatgttaaacatttctattttgatatctgtaaccctatggtagatgcgaatgttgcggccgccgtagattgtttcccaccgggccgcaacatttgcgtcaaagaggtacgcatacgtgttttggagcaacgcaacattgatgtccattttgcgccccggaccggaggaaataatgatcatccgttcgatttcgtggggatcatacgaataaatcccctttttaaataaaaaattgtagatcccggtttgctggaggccccgcacggaaataatccctgcttgctcgtattcccgccaacgacttttgagctcggtaaatcccttgctagaaagcgtatagggccaaaaggtagacaccgacatggagctgatagaaatttggatgtcctcgttggagggaaggggcagactccctccacggggaaacgcggcaggccccatatcattaatcgtatgaataataggatttatgaaattatttagggtggacaccacggagttaaagtcgtggcgctcgttttctgaccaattgctttcgataaagtagtgcccgttgttttgtatggtaagaataaaggcctttttattgataaagcgtattaaaataatagtgggtacacggaatgttttattgctgaatttttcaggctccgtggaagttatgtggtgtttggaaaccacggtgggacctgttttactataaaagaacaccaccagctgaggaatatcgggagtagccggaaataggtcgaaaacgttgcgcacattaatttgaatatttacgaggggcgaaattttaatcattgccgaggtgacggctaacgtgccgcgtgttagtctattcccctcgtacttggcaatgacttgttgtgctctggcatacgtaaagtttattagtttttgctctaggagtagcctctttttaagactggtcaaggatggagaaagagcaggatactgtttttccatttgtaagggagattgtaccaatagtttaaaggcatcgggggaaagaagaggccaatacttcataataaggccgtaatagagtaagtcaaattggtaattatcctccatggcaatggagatttggcgccgcatgggggccactagcgtgttgaggtctgccacaaagatgtgatgaatgttttttatgagctggaagctgtcgagcgcttccacatagagctcatctttttgactttccatagatgcgtcgatgttcagcccacccacctgttgaaactcctttttgtagtcgcgaatgtctagcgccaccccgctaccgcttaacaataggcgatacgttacctgaagcgcgttgttttgaaaaaagaaaatgtgttgtctataaggggggatccctgtggcaacgtaaattttttctcgaatgtctttaaaagtgtcttcagggaaaatactatactcgctatacatcgtctcaatttctggcatcatcacgtttgtctcctcgccactatcctccacaaaaagcttttcaaactcatctaaatcgtcgctatctccgcccaccacgtattgggaaagctttttctcccaatccttgccgtaaaaattttgtaaaatttctttatccttaggggttcgctgcaagtctttgcggcaggcctgtaacacatttgcaggcacggatcccaaaaaaataaacgtcttcgtgtactcattttccgcaggattataaagagtaactcgtagaggatttgttaaaaagtcattttggaaatccattatacccggtatagaaaataaaatttaaaataaaaaacggatgatatctatcatggaccgttctgagattgttgcacgggagaacccggtgattacccaacgagttacaaatctcctacaaaccaatgctcctctactattcatgcccattgatatccatgaagtacgatatggagcctacacacttttcatgtatggttccctcgaaaacggttacaaagcagaagtaaggattgaaaacatcccagttttctttgacgtacagattgagttcaatgatacaaaccagctttttttaaagtcgctactgacggctgaaaatattgcgtatgaacggctggagacgctcacccagcgtcctgtaatggggtaccgcgagaaggaaaaagagtttgcaccatacattcgaatattttttaaaagcctgtatgagcgacgaaaagccattacttacttgaataatatgggttacaacaccgccgcggacgacacaacctgttactaccgaatggtttcccgagagctaaaactgcctcttacaagttggatacagcttcagcactattcctacgagcctcgcggcttggtacacaggttttccgtaacccccgaggatcttgtttcctatcaggatgatggccccacagaccacagcatcgttatggcctacgatatagagacctatagccctgttaagggaaccgttccggacccaaatcaggcaaacgacgtggtgttcatgatatgcatgcgcattttttggattcactccacagagcctctagcgagcacgtgcatcactatggcaccctgcaaaaagtcctcagagtggaccaccattctatgctcctctgaaaaaaatttgctgttaagctttgctgaacagttcagccgctgggctcctgatatatgcacagggttcaatgattctcggtacgactggccctttatcgttgaaaaatctatgcagcacggtattctagaagaaatctttaacaaaatgagccttttctggcaccaaaagctggataccattctaaaatgctattatgtgaaggaaaagagagtcaaaatctcggccgaaaaatcgatcatttcctcctttttgcatacccctggatgcctacccattgatgtccgcaacatgtgtatgcagctttaccctaaagccgaaaaaacaagcctaaaagcgtttttagaaaattgtgggttagattcgaaggtagacctgccctaccatctcatgtggaagtattatgaaacgcgagacagtgaaaagatggccgacgtggcctactactgcattatagatgcccagcgctgtcaggaccttctggtgcgccacaatgttatccccgatcgcagagaggtaggaatcttgtcatacacctcgttgtatgactgtatctactacgcgggaggacataaggtatgtaatatgctcattgcctatgctatccatgatgagtacggccgtattgcttgcagcaccattgctcggggtaagcgggaacacggaaaatatccgggcgcctttgtgattgaccccgttaaagggcttgaacaggataaacccaccaccggcctcgattttgcgtcgctgtacccctcactcatcatggcctacaacttttcgccagaaaaatttgtagcctctcgggatgaggcaaaaagcctcatggccaagggtgaatctcttcactacgtctcctttcactttaacaatcgtctcgtggaaggatggtttgtgcgacataataacgttcctgataaaatgggattatacccaaaagtactcatcgatctacttaacaaacgaaccgcccttaaacaagagcttaaaaaactaggtgagaaaaaagaatgtatccatgaatcccatcctgggtttaaggaactacagtttcgccatgccatggtagacgcgaagcaaaaggcgttgaaaattttcatgaacacgttttacggcgaggcaggtaacaatttgtcgcccttctttctgcttcctctagccggaggagtcaccagttcgggtcaatataatcttaaactcgtctataactttgttatcaataaaggttacggcatcaagtacggtgacaccgactcattatacattacatgcccagatagtctttatacagaggtaacagacgcatatttaaatagccaaaaaacaataaaacattatgagcaactctgccacgaaaaagtgcttctgtctatgaaagccatgtctacactatgcgccgaggtgaatgaatacctgcggcaagataatggcaccagttacctacgtatggcctacgaggaagtactctttcctgtgtgctttacaggcaagaaaaagtattacggtattgctcatgtaaacacacccaattttaatacaaaagaattattcatccgcggaatagatatcattaagcagggtcaaacaaaactcaccaaaacgataggtacgcgaattatggaagaatccatgaaactgcgccgccctgaggaccatcgcccccctcttattgaaatcgttaaaacggttttgaaggatgctgtggttaacatgaagcagtggaattttgaagacttcatccaaacagatgcgtggagaccggacaaagacaacaaagcagtccaaatctttatgtctcgcatgcacgctcggcgtgagcaactaaaaaaacacggcgccgcagcatcgcaatttgctgagcctgagccgggagaacgcttctcctacgttatcgtggaaaaacaagtacagtttgatattcagggccaccgcacagattcctccagaaagggggacaagatggaatacgtctctgaagcaaaggctaaaaatcttccaattgatatattgttttatatcaacaactatgttctaggcttgtgcgcgagattcattaatgaaaatgaagaatttcaaccccctgacaatgtcagcaataaggatgaatacgctcagcgccgagccaaatcctacctacaaaaattcgtacaatccattcaccctaaagacaagtctgtcattaagcaaggcattgttcatcgacagtgctacaaatacgttcaccaagaaattaaaaaaaaaataggcatctttgccgacctttataaggaattttttaacaacaccacaaaccccatcgaaagctttattcaaagcgctcggtttatgatacaatactctgatggagaacaaaaagtaaaccattctatgaaaaaaatggttgaacagcgtgctactttggcaagtaagcccgctggtaagcccgctggtaatccagctggcaacccagccggcaatgcgctgatgcgggctatatttacgcagctgattacggaagaaaaaaaaattgtacaagccttatacaataagggggatgcaatacacgatcttctcacctatatcattaacaatataaattacaaaattgccacgtttcagacgaaacagatgttgacgttcgagttttctagtactcatgtagaactgctattaaagctgaataagacgtggcttattttggctggaattcatgtggcgaaaaaacatctgcaagctcttttggattcatataataatgaaccaccgtctagaacattcattcagcaggctatagaggaagaatgtggcagtattaaaccatcttgctacgactttatttcctaatacttcttaagaaactctttaaacaaggactttgcgtgctcgaaggtcctaaacccatgacccttatgatttgccaaaaaagcggtttcatcaagattttctaaccctttcacggatgaagaaataaggtgttcggcctcgtttgcccattttctatgatttttttttacctcgggttctagatctgtcttctccatatactcattgtggtcatatttttttttggagggaggcggggtgggtggaggaatgggtggaggaagtacacccggcttttccgcttcaaccgttttataaaaaaatagaagcataatacaaagaataaggactatcgcaaatatgataaccagtgtcccagtcgagggcattttgttatataagtaacgtttttttttattttttataattcgaatgaagaaccatgttgaatagtcttctacttaagggtgtttttatattatactgtgaatgagaatttataaaattcgaatatcgctatcatactgtttatctgagaaggtctcactgggtcctgtgatggagaacccgtactctgtaatgctggggtttataatgtggtcaggactgacgagcacatttctgaactgcgagagttctaggtttagacgcagtcgtaatagtcgctgtatatttgtaataaatattagattgcgtatgaggcgagtgtcaaagcgatcctttccgatttgtactaaggtgggcttttgtattccaactcccacttgtttaacgatggaccagggtccttcttcccgattttgttccgtgatataggtgagcacactattttccgtatatgaaatgggatgtcgcatatctctaggtgccatacgaacgggtgctcgtgcaactcgtcctgtgctaggacccaaccatcttggaacattaaaaacatatcgttccaaagtttctaaaaatgttctaagatcatttgcgaggtccgtaaagggtgtcattataatggcaggtatgtttaaaatattggtcataaaccattgagtcgcttcatcatcagtttgtagtatctgacggggatgtggaacagggattccatgtctattaaaaatgacgcctaattgtgctcgcactgttacgggcgtaatagtttgtattaagtctaacattgaattgggttgcccaatatttcctataaaattaattaaggtatctacccgagctcggtattgctgtgtccagttttgagcggctgcaatgacctcaggggttggattgttttgaatttcaggagtttgtatcatgttttcaacttctcttcgcatacttacaagctgctctctaattgtagttaaatccgctataagttggtttttaacaataatatttcttagaactcgaagtttctgaggtattcttatggtacggacaagttcatgtaaagcctgattgatgtattctgataagggttgcccccattgttcacgtccacgttgaatacctgcggccaaacccgggcctgcttcatcatagtcaaactgtgtggggtacagacttccaaataacactttattaaaaatttggtcggaaagaaacttaggacggcccatgttcagcgcattatctcctctaaaaatgcgtgacatatatccagcactgcccttgctcatcaattcgtttccatattgggtgatagagactgaaacataggggtttataagaagttttagcataaattctcgagtgttcatggggggacgattcggaatgtttaatacctctgcaatatctggctgaggagccgcggtgtttagcgatcgtaccttttcggccgaaatgccgtacataagacaagcaatttcttcaaaactatagtcatagttgtaaatattcgcaagtggtatagatcgcatcagcgcatttacattgataggtataatattcatatcaaacaagttaaatatgcgctcgcgctctctattagagccaagagtgcgtgtttggcctttcggcgatactattttgtgaatatgattgatttgctcctcttggtaagagctttccacgaaggaaatcacgtcttgcaatgttttacgaagcgaatacactgcattcatccctattcccgctgttataataggtttatcgtctctgttctcgctaataagattaactccaccaaaagtattttcattgtacatcatcactgttttaaaactacggatatttacgataaatcgaagagcctgaatagcatgggtataaaagtgttcgaatcgcgtgggagtaatttgttcgcgagcaaccactgtttcattataatttttcatgataagctgtactccgggcatatctgagagctgtaccggatcatttcccagtaattttcttgtgccgtatagtagtttaaactcgggggagccgctttcaaggtttgggtaaagaagaggatcatatacctcattattctctattcttaggtcacgcaaataatagagcgaaagtgaaaatggcataagaggctccttattgtaccgggacatatagttttgaatgaagtgttcttctgtttcaagatagatgggatggtcggtaagttcatgtaggacttccatggcagagtctgccagagtgtgggagcctctaatgatcccgtcgatcactgcgaccaatcgctttcgcacaacatcgctcgtattattttgtgcgtctcctaggggcataagcgtaacattgggacgaaatacgccgccaattccccgcagggccgcctgaccgacggatagtcctgtcataggaacattaatattattattatagtaaataacggaatcattattggcccccaagagccccatcagattaggacgagccagttggacatttgtgtattgtatgaattgttttaaaagctccccctgactaataagaatattaaacattttgttaaatagtggaagattggctctataattttctttaagatagataggaatttctgttaacgtggaaataagatgttgactcaggccttggcggttagtatccttaataagccgctgaagcatgagacccaaggacaaaaggagcaccgactgctctgtggggtcgcccctatggccaaaggcgatgttatcacgtgctaggtcgggatgagtgtaccccagctccatcaccgcttggctaaagttcccgttggcgaatgcattgataagattaagatatatttttccgctgggagcgtcataaaaccgggcaatgtacgaggctacaagctggttaaacaccatcatcatactacgattattttgaataccatagtctgatccatataggcgataacgtcgaaggttgtttgcggcatcattgacgttggcataggttctgggcgccatattgtcccagtagctaagagtattttcctcctgggcattgttggtacgaataagattggagagtctaaagtctcctagtaccacttgctctacacggagtccagagttattctccaaagcatcgtaaaatacgagtctactaaatactcttccatattgttcaaagcgctcagagggttggggattgttatttatttgaatattagctgcatctcttctttgtaccccacctcgaagttgcagtacattatagggttttgtaagcaagatgtaggttttattaatgatttggttaactccctccaggcctaattcaccgccaggaagcggccttcccccggcatcggtgggtggtttaataagcttgtcaatcaaatgttcttccagccagtaaaatgagccaggattggatctattttcgtagtattgaataatgtttttatcaatatgtgggcgtagaagattaagaaaatacttagtgtcggccatcaaagaatcaattaaggaaataagacccgtaaaatctaagtgcacttgagcggtgctagtttcgggaaagcgaacttgaaccattttgttaaaactagaggtcatttcgaagatattggtcaacaggagctgcatgattcgctgattgtctaccaaatatcttgcggctaattcttgctccgcacgaactcctccaccagcaggaatacccacatatggtacaatccaggcaaaaagagtctctgtgactaaattttggtcttggggtgcagtcgcattggtagttggatcaggatacaccctagaaagccgcacatcattttccttaacaaccaatcctggatttctaatctcagagatggccccgtgttttcttccgagccagtcaataagattggctcggttcacgttagcggcttgcgtttctcgtaaccattcaatgatgcttttttgaatcgtatctaggtctaaacctttaatgttattacgaaagttattaagaagtacgtaaatagcacttaataagttaagacctgtaataacggtttcatgaaacagaaatattttgttaacatctgtatctgccagtgactcagagccttgaataagttttgaaacaatttgaattttgtcggtatgctcctttttgagttcattgatagcctggcgaatgagttcctggtaggaaactttgcccaattcttgttgtaggctgggatcttcaaacatctcactaagctgtttcctaaatttttgtaccaagtcccactgggagttgggctgcagcattcctgtttggacatccacagagtctatattgtatagtgccgggcgccacttgggggtaggttgggttgaaggactaataaacctatcggagggaagtaattgcgaggattgtgtatagccgtcctcatcaggaagaatggagtagttggtttgattcatcattccgaagtcattcatagttcgcgcttcctgaacaatgcgttgaaatttttcccattcagtgcgtgtgataacgccgaacctgcggtttatttcatttacaaaatggataagcgcttttttggttgcttcttgttcaccatactctaagttaaagtgttggtaaatgacgtttatttctttgataagctgacgaatttcggtttctgagtagtcaccaatgttaataagctcaataggacgcataaagataatgcgaataagtcctgagaatattccttccagctcaggaagcatcgagatctgtacattttcatctctgaaggaaaacagtttttgataaaactcggcgaggcgaggaaggcggaagtaaagttccgctgcctcgggaattacctcaggttctagctcatcggcgcctcccagtatcatacgtgtgggtataagtttgtacacaggctcaggccgttcaaacatatcataaatgcctaatacaatgaaaatcttagcggccatacttttcagcatgaaggtgaagaagacgtcctcggtttcccagcgggttgatagggcgtcgttaactctcacagtagagaggtagacccgctgagccgcttcctcgggagttcgtgcaagcgctaccctttgccctccaatttcagattgatttagatttttaagtcccacggaaagcgcagaatgttgaatatattcaagcaaggttttatagatctgcaagggcgacatgggcaccatttgccgcagctcctctcccccaagcatgtctccaatccgggcaaaagcattgatgatatttttaagcgcctgaaagttggaaagagaacgcccgataaggtcgcgaatgtttttagcctggcttgctctgacgggacggagggtaccaacgcttcggccttgttggatttcagccgcaactttttcgtagtagtggcccgcaggagcattatccgtaaagacgttggagtcgttacctgcggaggtgggaaaactttcaaagacttgtgcaagcgtgtcccctgttgcctcggtgaaccatcgtcctataatgcgcacgccatccaacatctgctggactgtttgaatagaatctatgttgtttacaaacgttttggtaatgtttttaagataaaggtctagcccttccagagctcgatagaagcggcgttttacatcatactccagctcgatggcgcttacagctgccttccagtctgcttcctgggcacctccagggtttgggcccacgtgtcctctggcaagatctacagccggagaattaatgcgcgcatttttttccgtatccaactgcatgaggcgtcccgcaatagcatctccgagaatagtggcatagttttcctcgtaggattgaaactcctgtttgttatgcgttaaattggagtaaatctgggccacatagtagtaatacataaaagtattaattgcctggttgaggtcaacctgcgaccgcgcggccttgctgagcccaagctcttcaactgtgagagcagcaccgcctacccttgtgcactcgcagtcctccccgcctccgtactttttttgcacaatatcggtataaaaatcaataatctgcaacaagcgagagcaggagtcataaagatttttaaaattagggtcgattttagattgctcctctaaaacatttttaacaagcataaactgtgttaaaaaggtttcgcgttcttctcgtgcggctgcgttggtgtaaaagccgataagacttagatcaagcgcgatggtgcccatatcattaatgcgcgaaagagcatctcgaagcctcgttatgttcggcgtcaaggcaatttctttaacaagtttgatgcctatttttttcacattttccaaaaagtcgttataggcttgtgtgcttttgttcaaaaattccatgaggatgtgctttctatccagtctttgcgcttcaatcctcctatctagtggcgttttctcctcgtcacccccctttttggcacaactgttctcaagaattttgtggcgttcattaaaggtctgtcgcaacaggttcacggctttttcaaactcggcaatgttttctgcggagacaaggccactaaatcttttgaggtcaagctctttgtcaaactccgcccagtttttgctttgaagatactgttcaaccttgagtcctactttctggagagccttattaattttattcgcaacagatgcggcaatgcctagattacaaagtgtgtacgaaagcacttttccaaaatttttggttcccaagacactatttgtatcatttagaagtttaataatatccacctcatccgtctgcagtttatcaagttccttttgggtgggagttaaaatattgtcaataaaattcgttaaaatgttgatttgtaggttttgttcatttaaaagtcgacgatatactgcttcaatcatggtgactgcattaatgacttcttcattgggggctgctttggttacctccgtcaccatgcgctcgtgaagttgcttaatggcgtcgtttaacagcttgatgttttcaagtgtattttctatactgccatgtacatcaagatattccgcgcgcagtccatgagttagggagttgatgtacagaactatttgtcgacatatactggcggccccatcggtggtatctatgagcttatcctgacctaaatcaataaattcctggttaatggcgtctgcaatcattttacagacggtctcctgtttttccgcattttttacaaaggtggaaccggctcgaggatcgggcagttgttttttgatatctttaagaatatcttcgatgggctgctttgtgtctactttgaaccctattttggcaatcgccctgataatcccctctataacccgcagctttgctttactcgatacggagtctatgtgataatctttgatgtgttgtacaggatttttgtcccccccgccattaaaatatcctccccctgaaaaaggacgagtttgtctttgtatatgatcctgtaacttcgcatataaatttgcttctgatgaaagcggtggtctactagaggttgaagatccacggttacccattataataaaaaaaaataaagatttaaaactacaaatattttgctgtttatgaatccaatcatataagaataactaaaacattaaatgtaggtgagataaaagcttattttttttttaaaagtttaatgaccatgagtcttaccacctctttttcttcttcctttagaggggttccatgaatggtttgaataaagttatgtgctctaataaccttgttaaaatcaggtgcctttccatattgttcaatatgttgcacagtcttttgtgcaagcatatacagcttggagtctttaggttcctctgatgagggctcttgctcaaacaatgtttcaaaggaggatgtgcattcattggtttcattatcatttttttcatgaatgctttccgatgatgctgaggattctgtctcctcttcaaacagcacatgcagaatcatattccattcttcttgagcctgatgttcagtatacccttgccctgcatatatatgagcagatttcacaatatcatacttaacagtactgagcaatgtttttatagcggtcgtaacaattctaccgctattgataatctcaacagaaaaccaattatacaagctacccgcatgaaacacaacttgtgaagatgatcttaaatccgttttgaagatgacctccattttcatggatatatttaaaataaaatccattcaattttaaaattataaaataataagaagatgccctctaatatgaaacagttttgcaagatttctgtatggctgcagcagcacgatccagatttattagaaattatcaacaacttatgtatgcttggcaatttatccgcggcaaagtacaaacacggagttaccttcatttatcccaaacaggcaaagatccgcgatgaaataaaaaaacatgcctactccaatgacccctcacaggccataaagaccttagaatcactcatccttccattttacattcccactccagcggagttcaccggggaaatcggctcctacaccggagtgaaattagaggtcgaaaaaacggaggcgaataaagttattttgaaaaatggagaagcagtcctggttccggcggccgattttaagccctttcctgatcgccgactagcggtctggatcatggagtcaggctctatgcccctggagggtcccccctataagcggaaaaaggagggtggggggaatgacccgccggttcctaagcatatctcgccgtatactccgcgcacgcgtattgccattgaggtggaaaaggcctttgatgactgtatgcgtcaaaactggtgtagtgtcaataatccctatcttgccaaatcagtttccttgctgtctttcttgtcgctcaaccatcccaccgagtttattaaggtactgccgcttatagactttgaccccttggtgaccttttatctacttcttgagccctataaaacgcatggggatgactttttaattccggaaaccattttattcggtcctaccggatggaatggtacagatctgtatcaaagtgctatgctggaatttaaaaagttttttacccagattactcgccaaacctttatggacatagccgattcggctactaaggaggtggatgttcctatatgttattcggatcccgaaaccgtacattcctataccaatcacgtgcgtactgaaattttgcatcacaatgccgtcaataaggttacaacacctaacctagtcgtgcaggcctataatgagctcgagcaaaccaataccatacgacattacggccctattttcccggaaagtaccatcaacgcactgcgtttctggaaaaagctgtggcaggatgaacagcgatttgttattcacggcctgcaccgcacgttgatggatcaacccacctatgaaacctctgagtttgcagagatcgttagaaatttacggttttcgcgtcccggcaataactatataaacgagcttaatattacaagtcctgctatgtacggcgataagcataccaccggagatattgcgcccaatgatagatttgccatgttggtggcctttatcaacagtactgactttttatacaccgccattcccgaagaaaaggtaggggggaatgaaacccaaaccagtagccttacagacctagttccaacacggctacactcttttttaaatcataatctaagcaaacttaaaatcttaaaccgcgcgcagcaaacggttagaaacattctttcaaatgattgtcttaatcaactgaaacattatgttaaacacacgggaaaaaatgaaatactaaagttacttcaagaataactatgttgataccggtggtgtgttttacctgtgggtttcctattggaacctacgcggcaatttttgacaaggctcgtaccgagtatattaaaaccaaaatggacggaacattaccgcaaaatatcctattagatgcttctctccagattgagttaaaagatctcattacagctctgggaatcccaatgcgggtgtgttgtcgcactcatttaattactacgttggattatcgtaaatattattaatatctaaaattgaaaaaatatttttaatgttactagtaaaaatgactacacacatctttcacgcagatgatctcctacaagcattgcaacaagcaaaagcagaaaaaaatttttcttctgtattttctttagattgggataaattacgcacagcgaagcgtaatacaacggttaaatatgttacggtcaatgtcatagtaaaaggcaaaaaagctccgctaatgtttaactttcaaaatgaaaaacatgtaggaaccattcctcccagtaccgatgaagaggttatacggatgaatgctgaaaatccaaagtttttggtgaaaaaacgtgacagggatccctgtttgcagttcaacaaatataaaatctcgccgccgttggaagatgatggtctcactgttaaaaagaatgagcagggtgaagaaatatatcccggtgacgaagaaaaatctaagttgtttcaaattattgaactgttagaagaagcctttgaagacgctgtgcaaaaaggtcctgaagccatgaaaacgaaacatgttataaaattaattcaaagaaaaatttctaatagcgcggttaaaaacgcagacaaacctttgccgaatcctatcgcacgcattcgtattaaaatcaatcccgctacaagtatactaacaccgatattgcttgataaaaataagcccattactttacagaatggtaaaacaagctttgaagagttaaaagatgaagacggcgttaaggccaatccggataatattcataagcttatagaatcgcattctatgcatgatggcatcattaatgctagatctatttgcatcagcaatatgggcatttcatttccgctttgtctggaaatgggagttgtaaaagtttttgaaaaaaataatgggattgatgtgaactccatttatggctcagacgatatttcaactcttgttaatcagatcgctattgcttaaacaatttgctcaaaacaagcttataaacgtttcttaggtatgcgatacgtaaatcctaattctttaataagttctttttcagtagtgatttttagaggtactaaagtttgatttttaaataatccatactgatttagcttataattctttttttttaacgcggctcgaattcttattagataagaaacgggacccgtaaaatgaaatattgcgtatggtttttcctcggctaaagccgtaaaaagatcaagttgatacgtctttttttcccattcaataaaaagtacacactttcgttctccgcagacttttacagaaaaagaaagaccctttatgcgaatgttgggcaggacgtgttttaaaagttttttttctggaacaataataagaagatccacgtcattaagcattttctcttcgcgtcttaaactaccaacagcaacgatgttttttgataaaatttttataagttgtccattatattcaaacgcaagtcgggaacgtaagtgatttacaattttttttccttgaataagcgttaacattttatatttaatattaaaatcttttcattttatatattatatacgcaaaatggcacttgatggttcaagtggtggaggctctaatgtagaaacattacttatcgtagcaatcattgtggttattatggcaatcatgctttactatttttggtggatgccccgccagcaaaaaaaatgtagcaaggctgaagaatgcacatgtaataacggaagctgttccctaaaaacaagttaaaaaatgcaattatatgtatgcatgtaaaacgcgtaaataccacataaaactataacatgtcaatcatggaatcaacacttttataattttccgtaatatatttttcatccataatgatgtcagagtacatggtccctatgcgaggaacagagcccataagggtaggcgcggcaataccgtaaatgggattcacggcggagtcaaccgcagcatctgtcaagacctgaactggagacgacaaggccattcgcaacaacacgttggaaggctctcttgcattaagccctgccttttctagagaggtgacctgtcccgttcttgtcatgagatctgcgtacatgagtaaatgacgatggttgggacccttgtcccccataaccgttctaatttcactaataattttttgtcgggccgcttctatgccataaagctccatggtgtctcctatagaggacgacacgatggtgtatgggtcgatgttatcatcaagcattgcgccaaaaatattagtcccgtttgttttgatggcgtagatattatctagtcttaccagctttccctgggcatccacacggtggcgcataagcttaacaacattcgcatttttgatgcctggtattcctctaatcgtgctatttaatagtttatccaccacatttacggcgattttttcatccgtagccattcgggtattagtactgcgtctaaaggcgctttcccgtagatatatgcgaataatgatgggaatacctgaggccgtgttttccacagaatgcatgatgtaggtgttggggtgcttagctcttagactattaataatactttctagactaatgctttttaatatcatggttgttttatttaattccaagcggatacaccagtttgcaatatcctccgggggctgtagtagaggatggttttccagaaaatccgtcatccattccacatcacttgcaaaatcggggtacatcacatttttttttgtgcttgaatacgtttcgtacaataggtgccactgcagtatcaaccgttcgaacgttataagctctatgctgttagcaatttcttgcgcatatgttttatttgtttccacttccgggttctttagacgtaaaagcatttcagaggattgttcagcctctacgggcttcgcgctaaagatctcctggggccgcacaattcccgacttgttggttcccccggccacggaccggtggtgggagtccagcatatattgcgtcaagggttctgatacggactgcgccgccaggattcccaccgcctcaccgtagttaataagactttgagtatattgtagccttatgaggtccaggatggcactcatctgctcgcaggtaatgtttaatgttttaacggttgccagttcgatgcgaataagcatgcgcatcagagcggcagctcgtttaagataaacgggtatgggcgtttgcagtcgttcctgaatgttgttaataaacacgtatggaagatttttgcaaaacgttttgaccatcgcgtatttttgtagaatacttttttcgtcgaagggaagcacgccactggtggagctcagtagaatgttttttacgatgctggccacgtttaccggcacctgtctaacatctgtaagcagctggctgaaattaaaattttcgacgtttaggaagatctgtcgatatttatctctatcctttttaaggcgtgaaaattcttcttcaaacaagggggattgtatcccggtgtacttgaatttgtcctcaagttcctggtccgacagcatgatggtttcaaaccgtacggtttcgagctggcgtgcatcaaggccatcctctccgtacaactgctgcacaagacgagtatcgatggaaacccgtcggtaataatccacaatacaggattgaaggccaaagatggctttacggttggcatagcctgtggatgatgtcgataatgctttgttgatcaagtcgaatcttccattcatttccccaaagataaattcaggggaggtaaggcccgcaatatagctgttgcagatgaacccgtaggcctgcgcctccagggcaaacctggggtagtacaccagggtcctgccgaaggaaaactggggttgaatgcgttgtgtattaatttcaatttggccgatgcccgccatgatgtgaatcatattggggtttgagcccttggcgccagtggccaccatctgaaaaagcccattggtttccggattaatggaattcataatcggctttaaaatcctatcgggaaacttaagcgcattcagctgcaatttttcgtagaagtcatgcgttgtcaggcctataggcggcatgatgtctccatgaagcagccggttgtttatttcctccgactcaagcagcagttcattgataatttcttggacctcctgatgtgcctccggggttaggagcatgtcggccgtggacactgtgaatccggcgttgcgcacgtagttcagggcgagctgctgggtcgcaaatatcattttcaaggcctgctgcggcccatatctacgcgaaataaggtgatagattccaccggaggagcccgctccgacggcctttttgtcaaggacgccttcaatgagttcgccgttgcgtatttgtgtagagatgtcctgcttgttataatgcatgtagggcgcatacacttctgagtaccatgtgggggctcgttgataattgatgggggtctgcttcagtagcatagatacaaccgatttgccatccagcaggtcagttggggagtagttggcaaaacaaggtgggtccgtttgggttgtttgaaacaatcccatggcgtgcagtttgttcatcacatttttccccatgggggtgttcgtgcgtgtaagcaaaaagcttcccactgtggaatcctgcacctgcccattaacgggacccgaactcttcgtggaaatgaaccagtttcgcacagaacaaagtagttcggcctcaacgcggctcatgacgctccagggaacccagagattcatctgatccccatcaaagtccgcattataccaggcacatgcgctgacattcatttgaaacgtagaaatttttgggttttcaagaacgacaatccggtgaacccctatgctgcttcgttcgagagaaggctggcgattaaaaaacgcgacgtcgccagtgacgacatcacggtaaaggatgtctcctacctccagcctaaagtcttgtttgagaccctcaatatcgtgaacggattgtgttatttgcttatacactcttgaacaaccagggtactggcgctttccatttaaaaaatagggcattaatctattaatattataatgttgcactgtttccgcaacttgcagcgttcgtgcaaaggaaatgggatagccaacctcgtccaggtgaaggtctgagttcccgcagatggtggaccggctgatcgaccatacctggctgcccagtagggatttacgaattcttccctccttgcgaggaagtcttcgcatgatagagggagcggggcgggcccccatgacgatcccacgctttcccgtgcctccctgggttgcggtggtggaaacggaatccaacaaaaagttatagtaaagttgctgtatggtttgcaaattgcggtcaatatttaaaggtattttttggccgcgcacgatttgtaggtccttcgggatcagcagattctttcgaaccagatactggatcacgttattaatgtcgtgaaagctttgggggcctgacccgattcccaatctgatgccaggtcgtatgctgatgggggggatctgaatggccttaagcacaagtttttcgggatgggagtttttacttcgccccagttttacaacggtgtcgtaggttacgcgcgaaaaaatctctctgatgatctgcgggtacagtttgtcaatcttgccctgctgatccacccaaaaggtaaaataatcttccgagtccttaacaattttggggtgtactgccttacagacgtagcactgctttccttcggtttggcttgaagccgcttcaataagacgcttaggcctaataagatgctcgtacctctttaggtcaacaatgggagccccgcagttgaggcatataacccttaaccatcgtcgtatttcggcgatgaagagcggctgaagcaccggagcatgcatctgcagtatcccagggtgtcccatacattgcttgcgctggtgtgagcaagtgatgcatttataatggtgatcggtggttcccattcgcgcatcatagatacccccctcggcgggaagggtgccctcaaataaattagaaatggtaacctccataacgccttgcctcttatggtcattgtcaccggcaatattgaactgaacggcggctatttcggcatatccagcctccatatttttgctaaatacataataaaacttcaaatgttaaaaaaaaaataacatcggttggcatattttttgttaaaatcaaatgttaaatgatttctaaaacatttatcggttcacgaaaacctaccgcacgggcctgaagaggaatgccagttttgggggaaagctcggcatattccacggtaagctcttttccataaagatgttttttaaataaggcgggcgtgagtttttgaaaaagagcataacgatccgcgtacgtcaaatgcttaggagtcactacaaaccgctttttgtttggcagttcacaaacccataaaatggcgcctaagtcctttcccttttttccctgagtatagtccaccaaaataaattcagcgtctagcagcggtttcagcttggcaagatgcgctgagtggtagttgttgtatcccggctcatagggcccattggcattgcgtacgatggctccctcgtagccctccttaataaactgcgccttaagcctaagggcctcatccacattcttcacgctaaaattttcaacttggtggataaaggtaagatcttccttctgtttaaaaatatttgttaacagctgctgtctcttgttggaaggcatttgaagctgatcactccaaaagcagtcaaacacgtaaaagtgcagctcggaggaatctgtcttcgcattcgcctgccccgcgatccattgcagaggtttgcggtgtaaataaagctcgccatccaaatatactctcacgtctataaataaataaagctgtttgagctcttttttaatattgtcaagacctaaaaattccttttccgtgcgcgaatacaagagaatgcagccatcgccctgctggcaggccacagctcgaacgccattgcgcttgcgctgcacgatgggatctgtttcttcttcaaaaaatgtcttaggaattatattaaaatattttaccagcataggggggatcattcctctatttgtgtgggctccccgcttttgtctggcatggcgattatatttactaagggcatccttgaatgcctggtggactaccgttgtggcattttttttacccaagttttttccctcggtaacacgtgtcatttttgatatccgcaccgccccttcttccacaaaaaattttgtgaaaatttcagcaacggcgtcttttacatctgtggaaaacatctcatctgtgatgggaatgattgtgttgtgctgcaccacttgcacacaaataatccatgaggccttttttccgcttttcgtttcagactcaatcggaggaaaacaaaaaatgttgtttgaatattgcccaggaaattgatttagcatggttttaacaataaaataagcctatcaatttttttataatttgaatagttattccaaattcaatatggcttctttagataatttagtggcacgatatcagaggtgctttaatgaccagtctcttaaaaatagtactattgaacttgaaatacgttttcaacagataaattttttattattcaaaaccgtatatgaggcacttgtggcacaagagatccctagcaccatctcccacagcatccgctgcatcaaaaaagttcaccatgaaaaccactgccgggaaaaaattttgccgtcggaaaatctttacttcaaaaaacagcctctcatgttttttaagttttcagagcctgcatctctgggctgtaaggtctcgctggccatcgagcagcccattcgtaaatttatcttggactcctccgttctcgttcggctcaaaaatcgtacgacctttcgggtatctgaactttggaaaatagagcttaccattgtaaagcagctgatgggaagcgaggtctctgcaaaacttgccgctttcaaaacgcttctgtttgacaccccagagcaacaaacgacaaaaaatatgatgacgttaataaacccagatgacgaatatctttacgaaatagaaatagagtatacaggaaagcccgaatccctaacggcggcagatgttataaaaattaaaaacacggtgttgacacttatttctccgaaccatttaatgctaacagcctaccaccaggccattgaattcattgcctcccatatactgttctcagaaatccttcttgctcgtattaagagcgggaagtgggggcttaaacgcctcctcccccaggtgaaatccatgaccaaagcggattacatgaaattttatccgcccgttggctactatgtaacggacaaagcagatggaattagaggcatcgccgtcattcaggacacgcaaatttatgtggttgcagaccagctatacagcctaggtaccaccggcattgaaccccttaaaccaaccattttggatggtgaatttatgcctgaaaaaaaagaattttatgggtttgacgtcatcatgtatgagggcaatctgttgacgcaacaggggtttgaaacaagaattgagtctttaagcaagggcattaaagtcttacaagcgtttaacataaaagcagaaatgaagccctttatttcgctaacaagtgcagatcccaacgtgctcctcaaaaactttgaaagcatttttaagaaaaaaactcgcccatattctattgatggcatcattttagtagaacctggcaattcttatctaaatacaaatacttttaagtggaagcccacctgggataacacattagactttttggtgcgaaaatgtccggagagtttaaacgtgccagagtacgcgcccaaaaaagggttttccctgcatctactatttgtaggcatctccggagagctttttaaaaaattagcgctaaattggtgtccaggatatacgaaactattccccgttacacagcgcaaccaaaactactttccagtacagtttcagccatcggattttccactggcatttctttattaccacccagatacctcgtcattttctaatatagatggaaaggtccttgaaatgcgttgtcttaagagagaaattaattacgtcagatgggaaattgtaaaaatccgggaggataggcagcaggatcttaaaaccggcgggtattttggcaatgatttcaaaacagccgaactcacatggcttaactatatggatcccttttcctttgaggaactggcaaagggcccttctggaatgtacttcgccggtgccaaaaccggcatataccgcgctcaaacagcacttatttcctttattaaacaagaaatcatccaaaaaataagtcaccaatcctgggttatcgatcttggaataggaaaagggcaggacctaggacgttacctgggcgcaggggtaaggcatcttgttggaatcgataaggaccaaaccgcgctcgcggagcttgtttatcgaaaattttcgcatgctacaacccgacagcacaagcacgctaccaacatttacgtgttgcatcaagacctcgcagagcctgcgaaggaaatcagcgaaaaggtacaccaaatttacgggtttcccaaggagggagcttcttccattgttagcaacctgtttattcactatcttatgaaaaacacgcagcaggtggaaaacctggccgttctgtgccataagcttcttcagccggggggaatggtgtggtttaccaccatgttgggagaacaggtcttagaattacttcatgaaaatagaatagagctcaatgaagtatgggaggcacgtgaaaacgaggtggtcaaatttgctattaaacgtctctttaaagaggatatattacaggaaactgggcaagaaattggagtcctgttacccttcagcaatggcgacttctacaatgaatatcttgtgaacacagcgtttttaattaaaatatttaaacatcacggcttttccctagttcaaaagcagtcctttaaggactggattccagaatttcaaaactttagtaaaagtttgtataaaattcttacagaagccgataaaacttggacaagcctttttgggtttatttgtctgcgcaaaaattaaatattttttcataagaagtactacccaggttttaaagaaatagctaaaaatatcatatggatactgccatgcagcttaaaacgtctattggtttaattacttgtcgtatgaacacccaaaataaccaaatagaaactattctggttcaaaaacgttacagccttgctttttcagaatttattcattgtcattactcgataaatgctaatcaaggtcatctgattaaaatgtttaataacatgacaattaatgaacgactgcttgtcaaaacactggattttgaccgcatgtggtatcatatttggattgaaactccagtctacgaactataccacaaaaaataccaaaaatttaggaaaaattggcttctcccagataatgggaaaaagcttatttcattaatcaaccaagcaaagggctcaggaacacttctatgggaaattcctaagggtaagccgaaggaagatgagtcggaccttacctgtgccatacgggagtttgaagaagaaaccgggattacccgcgaatattaccagattctcccagagtttaaaaaatctatgtcatactttgacggtaaaacagaatataagcatatctacttccttgcaatgttatgtaagtcgttggaggaacccaatatgaatctttctttacaatacgaaaatcggattgccgaaatttctaaaatttcttggcaaaatatggaggctgtacgttttattagcaaacgccagtcatttaacctggagcctatcatcgggcctgcatttaattttattaaaaactatttacgatataagcactaggatgccgcattaaaatgccgcataaggtaatacactaggaatgccgcacacgcacaagaatacaatgtcgccggagatttattatctagtacacgttttatgtatgtacaatccgccttcatttaatatattgagcggatgtactatgtatttattttaacaaaaaaacattatttttttttaatcttcatcatctatttttataaactcagtaatatcaaaagtagcttgtggggtttcagagggttcaccttggttatcctccgtgaggataacatgttcttcaggttcgtcgtcactggagaacccatcatttaattcctcttcactcaacatctgtaaaaaatcttccaagctttcgctatcgttaaaatcctcatcatccataagaataatggtaccttcctcatcgtttcctccttgtttcgtgtctaaataggcctgcatggcatttgcaaaagtatcaaaatagggtgagtcagattgctgttccaaaatatggccttgcgtattaaatgtggttgcatcgttgttaaatgcttgcaaatacagtaagggatttatatccattattattaagcaaaaaaaatttaaattatttttcgaccgatgttaggtaaaattaaacaattgctataggtgttaagcaatgtttattgattttaagtactcaacaaccatgatgtaaatactatacagcacttttggatttttaatcaaatccagattaatactaacttcttttgtgatacagttcgtaataatagtatcctgctcatcgttttgtaagatttcttttaatatatttttttttaccgggatactaagcaattgattattttcttttaaaaactccttttgatattcaatcgtcttattcattgaatatttgtatataactataattacaaatgttcaatgaattgttattcatgtcgggagatggctatttaaaaatcatgtcctatttttctttgctcaataagcatccaaatattttcatggcgttttattaattgttcattattgaacgtatcacaaagatcattcataaattgcagatagtttattatttctttcaagagagtaacaaacattacttcagcagaacatataataggtaattcagtggcgttaaaagaattttgatcttgttgatacgccaatggcgaggacttaaggagattcgggggtcttgcccaaaaccctaggctgctgctcttgttttttagggcgtcataaagaaatgaaagcacattgcaaggcttaagccgcgacatctccttccccttgggccctttccatatttttagatctaagatctcatccgagcttatagagtaggtatagtaaagtttttcaaaaaagcatatctgcttgaagtcttttttagaacgactttcaagaagcatttctataatgttaacaagttttgttaggtttaaggcctgttcctgtgtaagctcctcttgcacgtgatagactgaaaaagtgtgcttaggaatgaaaatactccccgtggcactggcctgttgtctgccaggtatatagtacacgctgctgttagcaagctgtaccggcacaatttgccctacttctgcaacattattttgcgattcggacgagggtatgacaatagttacgggttcagtcaataggctttcgccgagaataatattactgtcatttttaataattttaacggccgctattaaatcaaaggcatttaagtaagaaacaacagcagaaaatcttacatgcatatatcctcttccgctattattcgtacgcataataaaacaaggggagcgttgtataacgccagtaatattaagaataaaactgtttttgaaacacttacccacataaatgttttcaagctccttcaaaagatgagcctccacatttgtacaaaaattggtaggatcatcaatattcaacgttgtctcaaaaattttttggtcgatcatatctataatatattctgtctatttcaatttaaataatatacgaataaataaacgagattattttattaaataagcaatggtgtatacactttgtatttactttgagatatactttgtgtatcacaacgtgccctaagatgtgtgcacaagtgacggcattttgtcgttaaaaaggtaaaaccagcggattccatcctgcattccatttggttgattacgagcctccatttctttttgcaaaaggttattgcgaatgagtaagcagagcttgatggcactaatctttgtaaggtttaaacttatgcccaattggtcagcaattttttgttgctcctcccgtccgcgtgtttcgcatacggctccccggtttagcatgcgaatatcagtaatctcgttcttttttaaaacctggataggtgggcggattttaaatttaagggcctttcccttgctttccatatagcctatgacaatgtcgttttcttttcgtttaacattaatattaagcatatagagcggaatttcatgccaggttttatcttctcgcgaggtaataagtcgcacggagtcctccgtggcatagcccaccagagtgttgtcatccccaggcacgtggcttataattttaaaaatgtccggaaatggctgaatatctttttttgaaaaagcgatgaaaaactttttataaacctcgacaagggcccccatacctgcaagattatctataataagtgcttctagcatcgtatagtgaaatgaagcgggataatggatgagtacctgctccattggctcatcctgaaaatccttctgaaacttttcatacaatacttgaaagggttctttggtctgcgagtgttcgaggtatttggtaatacggatgctgtgcatcgcgggaagctgaaaatcccgaatatatgtttcaatatctaataccggttcctttttatggttaagcaccgcagcgacgtacaaatgctcaggctttgccggcacatgcataatggtgcaaagacgattctgtatccataattccttgcactggttttttgagtagcatagagaaatgagcgccagcgcgaagttgtcctctgagaagagtttattatcgatggtaattccctgtatgagcttgggcgtggaaactgccttccatagctcggagtacgtccacacggggcgtgccataaacaaagatataataatattagaaattgtttttacctcttgctccccgtatccataggcctcaaaggtattgaggacggtggctccgacgtttgccggcgtgatggatggactaaggggcagactttccagcataggcttatcaatcttaatctggttggtgaacccatcaatggcgtgctttcgcagcgccttatccccctcctgtattaaaatgtattcttttaatttttgtgcgtacttagcaagctctggccctccatcgggtgttgtcgatacgtacaaatagattgtcacgttgcgctcactggggggaagctccatgtgtgaattttttcgcaccaccctcccaaatacctgaataagccggggaatatcaaggggcaatgacataatcatctcgtaccgcacggcctgaaagttcaaaccctccacaatcaccttggacccgatgagaatacgcagctggtggccttccaggttggacgaggcgttaaaaagagccaggcttcgttcgcgtacagcgggctctatttcgctgtgcagaatggtgaaccgtactggaataaactgatggtcgctatgtgtgtgctcatcgcgaatcgcggcgcagatggagcagcgggtcgttcccacaggggacgaaacttcatttaaaatgccattactttgtaaaatttcttgcaggataagaacccccgacatgcggacccgattgtggtaaattaaaattttcccccggccttgccgaataatggaaagaatgtctttcatcatttgagtgtattttccgctataaaaggccaatcccgagatgtgcgttggtggctgcagcgacaaaaagctgccactcacattaaagggggctctacgcgaaggctcaataatctgtaccccgttttccagaagccagtctgtgcttgccatagaaagggcagtgggggtttccgtcgagttaaacaggccgtaagccttgggttccgtttgttttgaaaattttgggttgggaaacaccatgtcataaatgctgtacgcattactcgagattttagggtcagggcccagctgtttaagcgtttcaagctgatactcagacatggggcattcgatgaaatgtaagtacggcaatgtttcgtctttataggacaacatctttccggcaaatattctttcaggataaaaattggtgttggtatccaacaaaaaagatacccttccggtgctgagtctttccacaagagccagggcgtcctttttccatttaacggaatgcccactgctgtcaaacagttgctggcgctggaggggctggccgttgggcagctcatgccgcggaaccaaaaggtttaacaggtcgacgtattccatgacactcccggttacgggcgttgccgacatgaagacggccctgggggcctggtgaggtggaaaggcatccaggacatactgtaaagcgatgccataattatttcgttcctggatattgtacacgttgtgtatttcatccgcaatgagcagtcctcccctaagctgctccatgattttttgattcacccggatgaggccgtttgtctcggcctcgctgattttttgcacgaactgagatatatcgttctcattcaatgtatcttctgcttcgtcagaacgatgaaacagagaaagcacatcaaagtttttctcttcacccttactcgtaatattgaaaagcttggatgcaaattccttatagccgtaaaactgaaaaaagcctccgcggtttctatcggttaaacggcgctttaacgtactaacgaacccatttagatgccgtgattcaaccgacgtggtgctgccagactgctttgcaatgtgaagaagccggtgtagctcagcgacctccttgtaagaaacaaatcccagctcaggacgtcttagcatttctgtttgaatgatggcgcgtgtaaagcctaccacaaaaatccagggcgcattttcaataaaattcatgtagtggttcatgaattggcgcgcaatggcaatcgcggcaatgctttttcccgtcccggtctgccagtttaataaaagacgcgagtagggtgtgttggggttttgaaagttttggacgaaaagctgggcattatgcaattggagacccttgatggaaggaaagggcgacgcgtaggggtcacacggaaaaaacgctcgcccccccttctcgcagccaggcccaccgatctggacaaaatgagcccgcagatcacgaatgagctctttttggtcgacaggaggggaaatcaacgacttaaactcctttcttcgcgccaactgctgcaaaaagtctgcggcatccaattcgggatacgccatattatcataaaaaaaataaacctttttatgaaaacttttatgtgattctgtattgcaattgttttttatgaatactgtaaataagcgtatcaacttgtttttctaacgaagaggcgttattctttttttctggatataagataataataagtataataattaagactaaacagcaggcaatcactatcaaactcatattatacttacttttttataaaaagtattatatcttatgaatgcgcaagttcagctaattgttcgtcgcttggaatgtgggactgcagggaggtggagtttttcctttttctaaagaatactgggaaatggtggtgaggctcaggttgttgtacatagtagctaggaggaggtttaggcatgctcgacttgcagtcaacagaccggttatagtaaacgatggcaacgatgataagaataataacgagcaaaatcaaaatgcctagaagaatcgcagttgttccgggatatctggcgatcgtatgggctaaaaggccttgagtgctttgtttaattccctcgcgggttgacaggttatgagaaagcagtggagacgtttcagtgtccatttattacaattgaacagttatattaatctcaaataaaatataacacaaaattaattatggccatgcaaaagttatttacgtatatttacgaatttattgaatatcgtaagatggtgctgttggaagaaaaggtaccatatgataagtttgttcaaatggtacttaatacaggattttttcgtattaacgcggagacgctgaatcacggaatcgtgtccgtgtttatctttggagcaaatggcaagtacgttcaccacggaggcgacatgagaacgcttttaacgaatacgcttaatgaaaaaaaacattatgaagaattaattttaatcgttgataagcccgttttaagcaaaaaaaatattttagatataatcgtcgagcagcgggctgcaaatcccacgattgtaataaacatatatccctaccacctgttctgcattaacattcccaaggtgagtgccattcctaaacataaactaattactcaggaggaggcgcaggagtttttaggtcgcgaatatctgcagccgcaggacctcatgcaaattagcgcgtcagaccccccggtggtctggctgggaggaagaccgggagactttgtgcaaattgagcggccctcagagacagctatgcacgctgttgttatccgctttatcaccaagtccaaaatttgagtcccgtgtttaaagatgacagacagctaagtaagcatatctgtaaaattgtcgatgtcctctgtggatagagcgctttcctctgagcagcagattttttcatacgtctccatgggagatggcgaggctttaatagtatgtaggtcacgtaagaactgttgtatgatgggatatttgtcttttaaaaactggggatgtttcataactggaattatttgaaagataaagaccttccatccaaagtagccaaccacatttggcatttcgggacacgcggtttcataaggcatagaatagtgaatagtgtactgatctttttgatacagcgtttcaagtagttggcgaaatgtttccgcgtcgagcgtgccaaaatcttgaggagcctcggtgtgctcctgtgtagagcagatcgtgatgattccccaggcaagcgggagcatggactctggagggtggatatccgtattggtctcattattcgatcccagctgatgaatgccgcacacgcgaaacatggcctcgacgtagatgcccatagagataggcggtgaaagggcaaggccggattgtatttgcggcatatagtaggagggcaccgagttttttatttttcggttgaatggggactttatttctaccagcacggggatgcgtttcgtggcctcatagcgtacgttgttaaaaattgttttgatttcccaggactgttgagtgtatcccagcgttaggtgacaaaacccatcggggctattactatgtccggggtatcccaaataggtcccatcaatatgaatattgtcacctatgacggtggtttggcagaacaactcaagcagatctttactaactcgctcaaaaagggttccccagctacaagcagcgcggttcaaattcttcttaaaaagatttgctttttccgccaagattatataatagcttttgtaagggtttaaacctaaaacgctagcaaggtcagagccacccacctgagtgcgacgaatagcatgccaggcatcggagcgctgctgaggagagtctttaaacaggcgtacaaaggtttccattatacttgttttaacaggaattcaatataaaaagtcaacacagtttgcaatttttccaatctcaagatatagccatacatttttttttccaattggcgaatatgtttaagctcatgtgtttcaatattagcatccggaaatttaaatgcataaagatgttcaaaggcctgatttatacacgtatcaaacgatttgtggtatgttattagcttcagcatgtgtgccagatcttcaagatggtctaaatttatacggttttccacgtggtggatcatgtctgccacatcttgagcccccatccaggggatcacaaggtactcccccttaaagatgattcgtcgtttttttaaaaaatcatgaaaacgttttaaagcttcaagaaaggggcagttgggctttgaccccaaaatgctgacgacgatatcctcgggcatgatgtattcgcagtgaggatagtagtttacggactctaattcagcggcccgccgttttatttcgtatcttgcccagttattcagagagtactccacgcctccgaccacaacagacatcctatctattaaaaaataacaataaaaaccttatgaaatctatgtatagtggccgctaaaatgtctatattagaaaaaattacgtcaagtccctctgaatgcgcagagcaccttacaaacaaagatagctgtttaagtaaaaaaatacaaaaagagctcacctcttttttggaaaaaaaagagacactcggttgcgattcggagtcctgcgtaattacccaccccgccgtgaaggcctatgcgcaacaaaagggactggacctctccaaagaactggagactcggtttaaagcgccaggacccagaaacaacacgggtcttcttacaaacttcaatatcgatgaaacgctgcagaggtgggccataaagtacaccaagtttttcaactgtcctttttccatgatggactttgagagggtccattataaatttaatcaagtggatatggtaaaggtatataagggggaagagctacaatatgtagaaggcaaagtggtcaagcgtccttgtaacaccttcggatgcgttttaaacacggacttttcaacgggcactggaaaacactgggtagccatctttgtggatatgcggggcgactgctggagcatcgaatattttaattcggcgggaaattcccccccaggtcccgttattcgctggatggaacgggtcaaacagcagctattaaaaatacaccacaccgtgaaaacgcttgcggttaccaacattcgtcaccagcggtcgcagaccgagtgcggcccctacagcctgttttacatcagggcacgcctcgacaacgtgtcatacgcccattttatatccgctaggattaccgacgaagacatgtataagtttagaacccatctgtttcgcatcgcataaactaataaagtttgaattctttataggaataaaaatggaagcgtttgaaatcagcgatttcaaagagcatgcgaagaaaaaaagcatgtgggctggcgccctcaacaaagtcactatttcgggtcttatgggggtctttaccgaagatgaggaccttatggcgttacccattcacagagaccactgtcccgctttgttaaaaatttttgacgagctcatcgtaaatgccacggatcatgaaagagcttgccatagcaaaacaaaaaaggtaacctacatcaaaatttcgtttgataaaggcgtgttttcttgcgaaaacgatggcccgggaatccccattgcaaagcatgagcaggccagtcttatcgccaagcgcgatgtgtatgttcccgaggtggcttcatgcttctttctagccggaacgaacatcaataaggccaaggactgtatcaaggggggaaccaacggcgtcgggctgaagctcgccatggtgcattcgcagtgggccattcttaccaccgccgacggcgcgcaaaagtatgttcaacaaatcaaccagcgcctagatatcattgagcctcctaccattacaccctccagggaaatgtttacacgtatcgagctcatgcccgtataccaggaactagggtacgcggagcctctgtctgaaacggagcaagcagatctttccgcctggatttatcttcgcgcctgccaatgcgcggcctacgtgggaaaaggcaccaccatttattacaatgataagccttgccgcacgggctctgtgatggcgctggccaaaatgtacaccctgttgagcgcgcctaatagcacgatacatacggcgaccattaaggccgacgcaaaaccctatagcctgcaccctctgcaggttgcggcggtcgtgtcccccaagtttaaaaaatttgaacacgtgtccattatcaacggggtaaattgtgtaaaaggagaacatgttacctttttgaaaaagaccattaatgaaatggtcattaaaaaatttcaacagacgattaaagataaaaaccgcaaaacaacattacgtgacagctgttcaaacatctttgtcgttatagtgggttccattccaggcatagaatggaccggccagcggaaggatgaacttagcatcgcagaaaatgtttttaaaacgcattactccatcccttctagttttttaacaagcatgacaaggtctatcgtggatattcttctgcaatccatttctaaaaaagataaccataaacaggtcgacgtagacaaatatacgcgtgcccgcaatgcgggagggaaaagggcgcaggactgcatgctactcgcggcggaaggggatagcgcactttccctgttgcgcacgggactgaccctgggaaagtccaacccaagcgggccctcctttgacttctgcggcatgatctccctgggaggggtcatcatgaatgcctgcaaaaaggtgacaaacattacaacggactctggagaaaccatcatggtgcgcaacgaacagcttaccaataataaagtgttgcagggaattgtgcaggtattgggtctagacttcaactgccattacaaaacgcaggaagagcgagcaaagctgagatacggctgcattgttgcgtgcgttgatcaagatctggatgggtgtggaaaaatccttggactgctgctggcctactttcacctgttttggcctcagcttattatccatggtttcgtaaaacgactgcttaccccgctgatacgtgtgtacgaaaagggcaagactatgcccgtagaattttactatgaacaggagtttgatgcctgggcaaaaaagcagaccagcttagtcaatcatactgtaaaatattacaagggattggcggcgcatgacacccatgaagtaaaaagcatgttcaaacattttgacaacatggtgtacacgtttaccctggatgactcggcaaaggagttgtttcatatttattttggcggggagtcggagttgcgaaaaagagagctttgcaccggcgtggtgccgctcactgaaacccagacgcagtccattcatagtgtccgacgaattccttgcagcctgcatctgcaggtagataccaaggcttacaagctggatgccatcgagcggcagattcccaacttcttagacggaatgacgcgggcgcggcgcaaaattttagccgggggggtgaaatgcttcgcttccaacaaccgtgaacgaaaggtttttcagttcgggggctacgttgcggatcacatgttttatcaccatggcgacatgtcgttaaacacaagtattataaaagccgcccagtattacccgggctcctcccacctctatccagtattcataggcataggaagcttcggctccaggcacctgggaggaaaggatgcaggatccccaagatacatcagtgtgcagcttgcgtctgaatttattaaaacaatgttccccgcggaggactcatggcttctcccctacgtctttgaggacggccagcgggcggaaccagagtactacgtgcctgtattgccgcttgctattatggagtacggcgccaacccatcggagggctggaagtacaccacttgggcccggcaactggaagacattttggccttggtgagggcctacgtcgacaaagacaacccaaaacacgagctactgcactatgcaataaaacataagattactatactcccgctgcggccctccaattacaatttcaagggccatttgaagcggtttggccaatactactacagctacggcacgtacgacatctcagagcagcgaaatataattactattacggagcttcctctgcgtgttcctacggttgcatatatcgaaagtataaaaaaatcgagtaaccgcatgacatttattgaagaaatcatcgactacagtagttcagaaaccattgaaattctggtgaaactaaagccaaatagtctcaaccgtatcgtggaagaatttaaggagactgaagagcaagattccatagaaaattttctgcgcctgcgcaattgtttacattcgcatctaaactttgtaaaacctaaaggtggtattatcgagtttaactcatattatgaaattttatatgcgtggctaccttacaggcgtgagctttaccaaaagcgtcttatgcgtgagcacgcggtgcttaagctgcgcattatcatggaaactgctattgtacgctacatcaatgagtctgcagagctaaatctttcccattatgaggatgaaaaggaggcaagccgcattctaagcgagcatggatttcccccgctgaaccacacgctgatcatttcccctgagtttgcctctatagaggaactcaatcaaaaagcgctgcagggctgttatacctatatactatctttgcaggctcgagaattgcttatcgcagccaaaactcgtcgggtggaaaaaataaaaaaaatgcaagctcgtcttgataaggttgagcagcttttgcaggagtctccctttcccggcgccagcgtatggctggaggaaattgatgcggtggaaaaggctattataaaaggaagaaatactcagtggaaatttcattaaacgctaccggtttatgatgtccaataggtgttaagcaatcagttcatcaacatttttttcaaggatttgaaaagtttggataatgttctggatacttttttctaaaagagttatcaaatcttcttgtgaggccttatgaataattgttaataccatttcttgcttatggggaacacactgataccccacaaagctaatatcaggaatcatttcataaatacatgtttttagcagatttccaatagtatgagtttcatcctttatcgtgataatagcctttgttttttcctcgtccaacgaaaacagcacaagttccggctgcggctcttcaaagttttcataaattttttgaatgctttggattcggccaataatgatccggcaggcgttttttaaatacgtgcgaacggcctgattgatatgtggcagcggcaccgctggaaagcaaagccccaggcggtggtgacgcgggtctgaggtcatagagctttgtttgtaaccactaagcgccatatattcttttttatctgttgggtactgctcaatatcgaggtgggaaaaatgtgttttaacggcaagattaaaggcggcatgttttcgtcctatgccctttttaatatagatatcctctataatcaacgattttccgggttgtaggaagccaatctcaaaagtaggattaaaaatcgggtatttaagcttagggcctgccacctgaatgagatcgcggctatagatggttttaacctcacagctattgtttaaactccgcagagcaaataccagtgtctcgtttttcgcataaatcggaatgaaattaatgcggtttctaataaattgttccgtcataaacaggtccgtggaatcctcgatcttatacccaccgggcttaatatctagcatataattgggaatttcatcttgcaagacccgcgacaggccgtgcaccgcggctctgctaatgcccttaaagtccataacaacattgactgggacgaggggcaactgctcctcgagctgaaatagtcttttggccgcatttttaataaaggggttggaaaagtctatcaaaaacggtttgatttccacgttttggaaaattttttccatttgtattataaatatatagatatatattcaaattatggtagtttatgacttgctcgtttctttaagtaaggaatccatagatgtgctacggtttgtagaggcaaaccttgcggcgtttaaccagcagtatatttttttcaatatccaaagaaaaaactcgatcacgacaccccttctcattacgccgcagcaggaaaaaatttcgcaaattgttgagtttttaatggatgaatataataagaacaatagaaggccctccgggccgccgcgtgagcagcccatgcacccattattgccgtatcaacaatcctcggacgaacagcccatgatgccgtatcaacagcccccggggaatgatgatcagccatatgagcaaatataccataaaaaacacgcgtcgcagcaagtaaatactgaactgaacgattattatcaacatattcttgcattaggcgatgaagacaaaggtatggacagcatgttaaaacttccagaaaaggcaaaaaggggtagcgatgatgaggacgacatgttttctataaaaaactaacgacgtaacaattaaacaaaaaataaaaatcattataaaatgaatcttgaatacgtccaagttgttcaaaaatttaatcaagtactcctagaacttaccaaaaaagtatgtaccgttgtgggcgggagcaaacccacctattggtatcaccacattagaagggtttgctcagaatgtccatccatgccgatgagtatgataggtccgtatctgaatgtctataaagcccaaattctaacaagggacaagaatttttttatgaatttcgatcccgcgcataatgagtacacctttatcattcaaaaactaaaagaagcagcccgaaatatgccggaagacgaattagaacagtactgggtaaaacttttatttttacttaaaagctacataaaatgtaagccctttattaattaaagaattgatactaactaataaatggccggtcgtgttaaaataaaacagaaagagctcatagactctactgtaaaaaacaaaaatgtgatgaatctgttccatgaaattataggctcaaaaggcaatattaattttagcgttgtctggcccaagtttaaaaaaatcaaacagagcgtttatgactacatttccactctttctgtgctggaaaaagcaagcgttatgcaaaactttgaagctgataagaaactgttggaactttttgtacaaaagctgtgggctgcctatgaaggctatttcaaatatcccgagattgaaaaatatgaggtggaaggccaggtaaatttcaatctcgtacctcagtgcgtcctcgaaaagtttagccagttgtataggataagaatcaattcagagcttgtcacactcatcctgaacagctgtgcctttatgagtaaatataacgattatattctcaaaaaagatccctacatactaaccataacccccggcctatgcttttcccccattcccaacttcgaggacctaaattttaaacatctttacaacagtgataaaaattctcagcatgacaaagagtttatcatgtttatattatataagctttatacggctgccctaggagtgtacaatgccatctcgattccagacatcgacgtagaagaccttgaaaatattatcctatcctcggtgagccagattaaaaaacaaattccgcgctgcaaagacgccttcaacaaaattgaatcttcggtacacctgctgcgcaaaaattttaacacatattacagtgactatgtgggctcaggctacaacccaaccatcattatggaacagtacattaaagacatatcacaggattccaagaacatatcaccacgcatttcctaccagtttagaaccatcatcaagtattaccgcgacatgatcgccaccaggcatcaaacgatggacccccaggtattaaacctcgtaaagcacgtcgaaaagaaattagatatgcttgatagagaaaaaaattagtatatatagttatggtgaatctttttcctgtttttaccttaattgtgattattacaattttaattacgactcgagaactatccaccacgatgcttattgtttctcttgtaacagattatattattattaatacacagtatacggaacagcagcatgaaaacaatacattttccatgccgcaaaaaaattcttttaacgaatcttataataaagacaaaaaatctaatacacatattccctaccagtggctggcgcctgaactaaaggaagctgagagcaagtactggtggggcaattatgatcctcatagcgagcccgttctcgctggcgcatcttgaatatcttcatacgtggcacgtcaccatcaaaaacattgcccaacaacacgggcttgatataaaggtggccattgtggtctcaacatcgcatttaaataattttttgccaatttccggggcgcttaacatcgaatgtataacctttcccagttgcggcatcaaggagatagacctcctatgggcgcgcattaaactatttcaacattactgcgccatcggtgcccgtcttttatggctggtaagtgctgacatcaggccccctgtttcagcgtggccagccatcgctgacagtctaaaaaagggagcagatgcggtggttattccctatccctcccgatggaacaatctcatacctaccgtcatcaaagaaatagttgtccaccaaaaaaaatgccttgtggcggtggatgcacgccaccttgatacagatacccagattgtgggggccgggatgggctgcatcgtcctaaccctaaaggcccttatggtgcgtctaagtattggcaaacagcccgttaagatactgtggcccgaccttcacggcactgccgagggcattcctctggagggggtggaggttggctggtttttaaacgcttatgcgcataaattaaatatacgctgcctaggggctgatcatattgcgcagcacttaacttaattctttatttaaaaagcccacgcatccagtagcggcctacattaagggcctacgcacataaatatacgctggctagaagtatgccttcatttaaaccattgaattatttatataatggctgtaaacattattgcaacaagagccgcgccaaagatggccagcaaaaaagagcatcaatactgcctgctagactcccaggaaaagcgtcatgggcattatcccttttcatttgaattaaagccttatgggcaaacaggcgcaaatatcataggagtacagggctcgcttacccatgttatcaaaatgacagtatttccatttatgattccttttcctttacaaaaaactcatatagatgattttattggtggacgcatttatttattttttaaggaactggacatgcaagcagtttctgatgtaaatggaatgcaataccacttcgagttcaaggttgttcctgtaagccccaaccaagtagagcttcttcctgtgaataataaatataaatttacatatgctataccggtggtgcaataccttaccccaatcttttatgatctttcgggaccgctagatttcccattagatactctttcggtccatgtggatagcctctccaatcatatacagcttcctatccaaaaccacaacctaacaacgggtgaccgtgtttttatttctggatataaacacctgcaaacgattgaattatgtaaaaataacaagatttttatcaaatatataccgccgctttcatccgaaaaaataaaactatatataccaaaaaaccgaatcagaattccactatactttaaatctttaaaaaacgtctaagtaataacatttttatagtctactcctagttccgaaataggctgaatttcttttttaagtcctttaaaccaaggatgtgatacaagactcttaaaggaaagccgcttattttcattaattgttaaacattccgtgataaactgttttcccgtctctgaaatgttctcgggaatataattttcccgtttcagaatatcatttaaataaaaattttctgcacgaaatctaaaaagattaaccgcgaccatacctatcgtccacacagttaaaggaagctggtaataataaccataataataaaattctggacacacgtattcccatgttccaaacatattatattggggacgggtttcgtctaatctaacagcgcttccaaagtcaatgaccttaatgatcttttgatttatgtctataataaggttctcatccttaatatccccatggataaagcccttctcataaatgttttgtataataagaataagctggaatattatttttttggcttcggtttcctcaagttttttaaagtaatgataatgaagtagatcaacactatttggaatatattctataattagtatatgatacatagcattttcggtatattcgataagcttaataacaccgggagtatcttgcagggctttcaacacgatgacttcatttcctggaatttcttttttagaaacgtacttaaatataatgggttgccctacttgatgacccaaaaagacgttatttctgccaccctcaaacatgggtctcgtcgcaatgaaatacatgtgctgcgttgtggaaatcctttccacctttgctgtaggataaaacgcatattgtgcctggggggtttttaacatttttttaagctgttgttccggcctggacatgttttattagctttatatataaagggttagaaggtttaatttcaatatatgccttaatgatgggattatattcgtaaaaggtatagcctaatcctacgtctttgtttttttggtaaaaaaactgtttgccctcgtaggatatgctataggcttttacttcggcttttacaagcggttggcagggattgggcaaacgtaaatcgcgttcaaagttttcatgaaaaagcaaagcatttgtgggctgacacatcagacagccgctttcgccgttgaaggcacattcaatggccgccctttttagtaaatcgcggaaagcagaattaagatggctcttttcaagccccctttcgtgaaaacgctcatcaatcgttttttgttcctgactgccttcgggaatactataaaacattttttgattagccaccgcgatgtacaaaaaaggctgcacggttttctcctcgggcggtagcgcatcgtggctaccaatgcgtataatgcgcgccttcacttgatcctctcgggccttatcccagtacggctctaggatatgaacctgccgcccgtatttgagatccaatccctcagctcccgttttagagacgagtaaaattttaataacctctccgtgtatattcagcggcgaattccaaacctgctggatcatgtcgcgctctttagataaaattttccctgtaataagcgtaaatcgtgttattttggaggacaggactaacgtatgggtcggcccatcttcggcaaagtttttcaccataagatctttcccatccttatgaaggaggatggtgttgtgcccttcttccaatacttttaggggctgaaggcactggtagccctctatttctaaaaagcgggccacgacgtgaaggcccaattccacaaactgtgagtaaatgagcacagggcccggagacgttttaatattttttagcatgcgcactattttgggactagaattttctgtgaaggcctctttgggcagctgctgaacagcctctgataatttttcatcctcctttactgttagcatttcggacgcgaagatgctgatcatacgggaacgcacatagtaggaggagcctgactcttgctccgatcctggcaggcagagggcggcggcattaattttttcatacattcctgagctggcgtgcttttccgcgttttcaacgtctcgggccagcagatattgcctatactgctcgggtgacatttcaaccttttctataataagaggaagctctgtggggaatagcttgttgagctcattctggtttccagcgtagcttatcatacccactaggcggtttagtagtttgtccgcgtttaaagggctattcgttgttttattgacataagcggtgtagaatctttcatagtgaagaggtaataagattcgcccgcttagcatattaaaacagggcaccatttcaaaggggtccttcgaacacggggtgcctgttaaaaacagaatacgaatatttttagcttgcataatattattgtacagctggcgggcatttgttttatcattggcgctattgataattcctctaaagaggttgtgtgcctcgtcaacgatgagcaggcaaccgtttagggaccctcccgcctttatgatctgctgccccatgttgtaagcgtctagggacacaaacctgaagcgccgcgagattttttgtagctctttggagtgatccgtcgtttccggatataaaagtttaataagctttagcaaagactgttggaagtttgagtgcaacgacttgggtgcgatcagaatcgggttgtaaatatgtgaaagtgagatggcaagcgacaggctcaaaatggttttccccatgcccatctggtgatagatgaggaggccccgtgtgttttccccctggcctatcccaaatttaggatccgaaaaggcggtgtaaattaaaaactggtagtatttcagtgctcgtgcaaaacgggcagtgagtgaggtgtctttgctttcctgaagctctttatatttttcatatacctcttttaggtatgcttctatttggacggggaaggaggtgttgttgtgcacgcaagacatgactcgttataaggatcccatattaaaacttcattagaagaatagggctgctgatagctagcgctgcacttaaaaatggggtagccctttttcttgtaaatccggtgcctgtcgtagacctggctagaaagcgggcttagtgtatctttaatgtccacgacgatgcgtacctttttttcatccgatccctgccgggtaatacgtcccaagatttgctccatgttgtttctgcggggcgttgccatgatgatcgatgtcatatgcttgaaggaaatgcctctacgcccgtagccataggtcagcaagataatggaagcgctgtgtgcctgagaaagagcggtatttgaaaccccgccgcataggagcgccacctccggaacgacaatttgaacatctttgaattctttggaaagcgcctgataaaaaatttctaaaagtttgcgaaattccacgaaaatgatgatgccatacggctcatcggtcccccatttgtgaggctcagcggtatgcagggagtaaagccgctttgcctcatttacgacaagttgtatacgcgaaggatcttgaagtagtttatcaatggtggcaatggccgataccttttcattaatacacacagggctaacgaagtcaggatgtccctgatattcgatttccctcacgtacccggaaaaggttgtggtgggacttacagtcctctggggctgtcctagatggtgaataataatcttgtccataccatcgggccggtccaggggtgtggcggacagtcctaatatccgactaagttgtattttccaaaaaattttgtaattctccggcgagtgtaattcatgtgcctcatctaacacgactagaccaaagggctcaaagaactgctcaggcttcttgcgcagggtattaatgattcccacgatgacgtcgtactctttgctcgtcatgtcctttttcttgcacgctgcattattgtaagcagctacacgtaggtggggcaggagcaatgttagctcgtcgatccactgtatttgaatcgccttggtgggcacgatgaccagggtagggtacaaaagtttttgaataatgctgatcgcaatacgcgttttccccaaaccggtatttagatgtaggtaaaagcgcccatagggggacaggagctttttatgaatcttatcgaccatttcttgctggtagttaaatagtggaaattctgtttcaacgcatgggagggcccgcagcgacacggggcgcgtcgtgtaaaccatgttaaacatttcaaactgctttcgcagcaatatgggaaaataaatgtattccccctgcagcgtgaaggcagtttcctgtcttatggctatgtgctttggctgcccaggcaatgcccgcgccgtaacggtgagcgccttaagaaggcgcccgaaatcatgttgtaatttactttgtagcttcttataatttattcctattccagcaaaggatataatggcctccattctcgcactggacgggctatatgcagaggttccaaaattcttaccagaggcgttacgagagggctgtgctggcaagaatcctctaagcttttatattcaacaaattttaaatttaatgggatgtgacggtaacgagtaccatgttctttttaccggcagctctgaggaagcaaatactcatatgatcatggccgccgtgcgtcgccatttgctgcggacgcagcaaagacctcatgtcattatcggagcagccgagccccctagcgtcaccgaatgtgtgaaggcattggcgcaggaaaaacgctgcgtatacaccatcatccccctaaaaaattttgaaatagatcctgttgcggtatacgatgccatacaaagcaatacctgcttagcgtgcatttcaggcaccaatgctgttgtcaaaacgttcaacaaactccaggacatcagcaaagtgttaaaaggtattcccctgcactcagaagtgagtgagcttgtttatcaaggatgtattaaacaaaatcctcccgctgatagtttttcaataaatagtctctacggcttcttgggggtcggtgtcttgggaatgaaaaaaaaggtcatgcaaggattggggccgctcatttttggaggggggctgagaggcggaagccctaatatacccggaattcatgccatgtatagaacgctaacccagcaaaggccttccatgaaaaaaataaatacaatacataagctgttcatgaaaactttaaaaaaacatcagcatgtgtatctacccatagggggggtgtctgcagaggacacgtctgcagaaaacatatctacaaaagacatacctgttgaaggcccgaaggaactcccgggctatattttatttagcgttggtcgtcgcgccgaagaactacaaaaaaaaattttcactaaatttaatataaaggttggccgtattgttgacttacaagagatactatttcgtatcaaaataccccaaaaatactgggagacattattgttcatccaattaagagataatttgaccaaagaggatataaagagagttatggttgttttgatgcatttagataccatcactcctcgtggctctcttcctcctccgagctactcttcttctttttcttaatcgtttttgtttgttctataataagggaaaagaactctgtgggatcttgttccccgtacaggttatctgcgaccataaggatgcttagaatggtaaacaggtgagaatacataagggtttgcgttttaagaaaaccctgacgttgaatcataattgaaaacaccttgcaaagccgactcatcagttgttctgtaatggcgttaagcattttctggaatttttcttggttttcgggtgtgattttatattcatgtagaaagtgtttcacacctgaggagaagaatctttcctccttcgagagcccatctttgatgatgggaagttccttgatcagggcaaaccattcctcctcttgggcctgcggattctgaagatactgatggcagatatggtttagaatggtgcacacgtagctaataagctctgagctgattctttggttggttttcaaatgttggcgaaagtagtttttcaccgaagtgcatgtaataaacgtcttcattttcttataatatacaacagtatgttgagtctttaatttaaaattacaaggagttttctaggtctttatgcgtataggtgtttctttgtcgtaaattttcaatagccgacattgtttgtgaagcagtgttctgagtagtgactgtcgtgtaaggctcagtcggatgaacactcgcggccgcaggtgcggccgctggcccgccagttgccatgactagtctgtccgtaactgggttgtccgtgactggtttgtttgttgctggtctgtttgttgccggtctgcccgtgactggtttgcctgcacttgctgtagtcgctccagccggtttagaggtacctggttgtggagtgacttctgcccattgctgatcttgataaggatttataaactgtatatcttcctcctcaatagcggcagcagcttttttctttcttgaagagaatagatagattagaacgatgataataatgactaagaccacgatagcaatgagaatagtatacatatgtgtggagaagaagcttggtggggtgactggtgacaaacactcaccataatgccgcggataaaccggttgaaaaaattcagaatccatttaagatactattataaataatatataaaaatgttgtggcgcaatgaaattacagaatttatggaccaactttccaagtattctcaagaaatcttaaaaacgtttaagcaattgcgtcctagtgaatataaacaatacaatgaatttttaacacaagttacaccgttgctgcaaaaaacctctgaaaaaattccagagttggttgaccatatattcaattacctagacaacgttgaaaaaatttgtgagctcctggtgaatgctagctcaattattattagttcaaaaatacgagaacaagtaaaacacggaatgagcttcagctataaagccgacctcgactccttggcggacatcctctctcaaaaacagtacgtgcttatgcatctttcaaaaaatattgcggccgagtattttaatacgtgtttaaaccaaggaaaatccaagttagatctcaaagctgcctctgtattttatagtagccgtccccgaacggcaagctcagcagaactctatagaaaaatgctatacgcctatggttcaccgcaggaaattaattattatactgaaaaagctcgaaataagacgctggacgtggaggagagcgacagcatggccatcatcgaacgaacggcccgacacaacctttcccttatgcacccgctagaagccatggggcttacctttggggcaaccaacacggacgccgacccggaggatctgaaggacaaaacggtgataaatttaacgctcccgcaggcaacagaaagcatcacctaccatcttaaatccctaatgcagctaaaaaaagtaagtacggcttcaggactaaatacaaacattttgaaagcatttgataatattatttccacccctgtgaaaaaaaataaaatggcctccaagttggcgcccgggatggatgtcgtgttcactagcgataacggaaaaacattttttactaaaaacattttaagcaaaaacatgctagcggggcccaaagagcgggtgtttgcatataataatctcattagtaatttaaataattcctgtttcatacaaaatcacaacgattttttaagacagcaggactcttggcccttctatgacgcgcacaattttaccaacaagtttttaatgcagcctattttctcggggcagacccgtcctcggcttcagggagccatggaggcggcgcatgtggaaacgcatcttacggcatttttacaaagtattcagccctctaggccacaagatccctctgttttggcttcccccaagttatctgctctaatcttgaactaaaaacggctcctttcttggacttaaatgatggtctgccagtttttgaaataacttagagaactatgaagattttcatgaaatttaaattagagatttgcaaaggttacttgcggtcattttctgttgatttaaataattattagaatagtataatgtctgaagatattcgtcgtggacctggcaggccgccaaagaaaagggttgttcccaactttgagcgcaagggcattctggaaaaaccagttcggccacaaagccgtctcgagttttcctatgataacccgctgatatttaaaaatctttttatttattttaaaaaccttaaaagtaaaaatattttggtgcgatgtacccccaccgagattacctttttttcacgtgaccagtcgcaagcaagctttgttattgccaccatcgacggaaaaaacgtgaaccattattacgccagtgatgtcttttggctgggcatcaatagagagctcgttgaaaaaatgtttaacagcattgaccgctcttttttaaaaattaccatcgttcaccgctatgacaagcctgaaaccctgttttttatctttacggattttgacattgacaaggagtgcacgtatcagattacggtctcggagcccgagctcgatatggaccttatcgaaatggaaaaaagcatcagtgaagaaagactcaagaactatcctctgcgctgggagtttacctccaagcagctcaagaaaacatttagcgacttatcaaactacaccgagctcgtgaccattgaaaaactcggcggcgatacgccgctgcacctgtatttccaaaagtttaactccatctcataccacgagatgtataaatcttccaacaagatcaacctgacctcgaccattcctaagtcgcaggtgttccagataaatgttaaaattgctcacatcaagtcgctggcctcggctatggtcaccgacaagatccgcattctgtgcgaagaaaatgggaacctaatctttcaatcggaaatggatgcccttatgttaaatacgattaccttgaacaacacgatatagttcggtaacattagatgttctaatatttagcatctaaataatacgctgtagtccggtcagggttgcgtcacagttttcccatttttttgcgtcgtcggcggtggccaccgttgccctatcatttacgcccggtaagacaaagctaaaggcgttcagcggggcttggcaatgcccgcccagcgtgaaggagctcggaggattttgcgcatcccgaaatcccttagccatgttgtttaacacttcggttacgtcaatcgagtgaagggatcccttgggatccgtgaatgtaaagatgcagtttctaaagcgcatgtatgcgatggacgattcatcgggggttttgaaggtaacagtgttccccttgctgtacttaaagggggaccatccggtaaaattataccaaatgaaagcaataataattaaaataaccaacacaatagttatagacaacacaaagtctgtagtgccgcccattattaaataaaaatattttagaccgccggcttaaaatttacttattgctcatggcttaagtctattttattcatagcttaagtttattgctcatagcttaagtctattgttcatggcttaagtctattactcatagcttaactccattactgatcatgacttaaataaaaatattttgcccgcttaaaaattgtttaggtttgaaaaaataagagatggagggggcaacttatcgtcattgtgtttacccccactggaaggcatcaaacggtaaataattataagaatcaaaatgattaatataagggttaaaaaaggatgattcatcacattaattaaaaacgtatttataacgctgttgcagttgaaattttggtataggtcggaaatattgcccgagcctccgtattctgcaatgttctgacatatggtgagtccggaggggcactgcttgttggtcaaaatatttctatgctccgttgttttataggcatttttatttccattacacggagcaaacgcacattcagcccatagggtgccggagttcacacaggcacaatactggctatacgcatactcatcctttgagcacaatccctgtttatcgcatatgctcccaataatattgtcatcctccgccgtttgttgatttgtatgcgagcgtaaaatagcggcccaggccttgggctcctttttttgcagctcggaaatcgaagggcctgtacagctaaagtcgacccaaatatcattgcatttcgtggaaactggcatgcaagacataattgaaataattaataagtatatatcatggcaacaaatttttttattcaacctatcacccaagaagctgaagcatactacccaccttccgtgataacgaataaacggaaggacctgggggtagacgtatactgttgctccgacctagtgcttcaacctggactaaatattgttcgcctgcatattaaagtagcatgcgaacacatgggcaaaaaatgcggttttaaaatcatggcgagaagcagtatgtgcacccatgaacggctgctcatccttgcaaatggaattggtttaatagacccgggctatgtgggcgagctcatgctcaagatcattaatcttggcgacaccccggtccaaatatgggccaaagaatgtttggtgcagttggtggcccaaggtgaccatgtgcctgaccatatcaacatcctaaaaagaaaccaaatatttccgctgtttgcgcctaccccaagaggcgagggtagatttgggagcacgggcgaggccgggattatgagaacttaattttattttttttcttaacataatgggaggctctacaagcaaaaattcctttaaaaatacgaccaacattatcagcaattccattttcaatcagatgcaaaattgtatttccatgttggatggcaaaaattacataggcgtattcggtgatggaaatattttaaatcacgttttccaggatttaaacttatcactggacactagttgcgtgcaaaagcacgtaaacgaggaaaatttcattacaaatctttcgaaccaaattactcaaaatttaaaagaccaagaagttgcgttaacccaatggatggacgcaggacatcacgatcagaaaacggatatagaagaaaatataaaggtaaacttaacaaccacacttattcaaaactgcgtttcatccctgtcgggtatgaacgtgctggtggtgaaggggaatggcaacattgttgaaaacgcaactcagaagcagtcgcagcaaatcatctctaactgcttgcaggggagcaagcaggccatagacaccacaaccggcatcactaacacggtaaaccagtactcacactacacctcaaaaaacttttttgacttcattgcagacgcaatttcggctgtttttaaaaacatcatggttgcggccgtagttatcgttctaatcatcgtagggtttatagccgtcttttactttttgcattcacggcaccgccacgaggaggaagaagaagctgaaccgctcataagcaacaaggtattaaaaaatgctgccgtttcgtaataatttaattaaaagtaaaaaaaaaaggtattgttatagtgatggcagattttaattctccaatccagtatttgaaagaagattcgagggaccggacctctataggttctctagaatacgatgaaaattccgacacgattataccgagcttcgcagcaggcttggaagactttgaacccattcccagccctaccacatcaacttccctgtattcacaattgacccacaacatggaaaaaatcgcggaggaagaggatattaattttctacacgatactagggagtttacttcactggtccccgatgaggcagacaataaaccggaagatgacgaagaaagcggtgcaaaacctaaaaagaaaaaacatttgtttccaaaattaagctcgcataaatcgaagtaaaaattgaagcgaaaaaaaagtagaaaaaaatgtttggagcttttgtaagccaccgtttgtggtcagatagtggttgtacgactacctgcatcacaaacagcattgctaattatgtagccttcggcgaacaaattggatttccctttaaatcagctcaagtatttattgccggccctagaaaggctgtgataaatattcaggaagatgataaagttgagcttttaaagatgattgttaagcacaatctttgggttgttgctcatggaacctacttagatgtgccctggtcccgtaggagtgcgtttgttacacattttatacaacaagaactacttatatgcaaggaagtcggtattaaaggattagttttacacctaggcgccgtggagcctgaacttatcgtggaaggactaaaaaaaattaagccggttgagggggttgtcatttacctggaaaccccgcataacaaacatcatacatataaatacagtacaatggagcagatcaaagaattgtttttacggatacgaaataccagattgaaacagatcggtttatgcattgatacggctcacatctggtcttccggtgtcaacatctccagctataatgacgcggggcaatggctgcgctcgctggaaaacattcactccgtgatcccaccaagccacattatgttccacctaaatgatgccgccacagaatgcggaagcggtatagaccgacatgcaagtctttttgaaggaatgatttggaaatcatatagccataaaataaagaaaagcgggttatattgttttgttgaatacattacgcgtcaccagtgtccggctatattggagagaaacctcgggtcttccatgcaattacaaaccgctttaaccgcagaatttactacattaaaatcgttattaaaataaggatgagttttagcgaatgtcccttagttattagtgcatgcaaaaaatttctacaaaagcgcattacaatagagaatgaagcacttataaatgccttaataaccgctttagcgcaaaccagcacgttgaatgatctttgtttattacctattcaaacctatttgcttagctataaaaatgcttttgagtggatacacttcgtatgtattgcaatcaccactattttagacaataagtataactggaaggactgtacggtagatattaattatatttttctccacgtaacatatatttacaatattaaaaccaaggaatacctagactactgttcttaaactttattttttctatatttacgccaaggaatatttaaatttttttttgaaaaaaaacaatatatgtagataaaattcagttacatgatataagcatatgtgtaaacaacatatggttacgctttatgagataaatgcgcatattatatgtaaacaaaatatggttatacttatgagataaatgtattttaacgtatacttgtgataatggatatatgcttttattaaaagaggctgtatttattataaatcttgctaaggataccattgtcaacatatatcccgtgttggacaaattgcgtcgcgatccagttcttttttttttgattttgtttaacgctatcctttttgaagggatggttgtccaccatatttattcgatgttcaatgaataggactgcttcttcgtaaggcagtgaaggtcgtttcaacactccttgaacgatggacgtgttttcttggatccacttaaaaagcacgtggcattcaaaaacaggacagtgattggatccttggatatgctttggacagccaatgcttgaagaaatgtagtcccttttctttaggacaagcttctccacgctggggcaacagagatcgttcaagttctggacggtcgcatttggaatgttgaaacttcgtacccattcaccctcgggccctcccttatgaagaaggagtatttgctcatggtctttagtaatcttaaccaaatgttggaagatcatttttttacctgctttaaaggcctgaagggtgtcggttggcaaggctattgaattcgggagtgggctttcatcaagcgtgaaatggtgaatgtggcgcgactggaaagaaaacgaccgttgatttattttttcaaagattgggtcgattccgccatgaaagaacagctgcaagattttagaagccgtatttttttcccaataaaaaatgactacttctcgcgggattaaaatcgtctgtgtcccattttcattatataattggcccataaagccatcaacgtcaatcaacaccaaaagcatagtatagagagcttttagaaccggagttcgttaaaaaaatacaaagttcgtttaaaacgtaagatgttactaaaaaaatgtaacgtttaaatgataataataccacatgcattaatgaaaaaaacttttaaatttttgttttaatatttgcatgaaaatggaaacatttttagtctgtttatttcacaatgctgatggtttacatcaacagattcaggaaattttgtatttattgcggatgcatatttatgagacaaatctttacttaaagcaggaactatcacggcttatatatccaaataggcagctttcttttgtgttacttatgcccctttcccttctaagaaactgggatgacattgaatatttgacggacgttgtagacgataagcagactctacattacgcggcaaatttgctgacaaactacgttctacatctatccatgtttcaaaagctgacaaaaccatacttccttttagcggtaaagcgggtcagcgaaaaactcaacaaaaggcagcgacattcattttacgaggtattggtaacctctgaaaccttgaataattatgaaaacctatctaaaaacattttaaatacgttgatgtttgccgtgcgctacgtatttaaacctacaccgaactattcagaaattctcgcagagttggaaaaaaaaaataaaattcaccatattatttttaatatggtaattgcggattttgcgcaaatccgtaaacaacaaatggataaacatctgtgcgaaacaaataatgagcttcgtcaggaatgtaaagaaactatttttgatttaaaggtggtaggaaatgtttagccaataaactcatgcccgcattttttacaggtacaaaatatcgtggaaggctcatcgagggcgcgtgtttgtacttctctgtaggtgcacatacgctgcttgcagttgggacacttataaagttgtgacgtcttttcggcgactttttgctgcgaacgtagagtaatttctgtcttctcctttaaggcggcagagggacaaagctcggcgaacgtcatgctaccaattgcctccggttttagctcgccagaaattagcttattaagggcatcgttatcctgctgctggtgactttttttttcgcaattaataatatgattgatcgtcccacaacgggttgaatattcttctaaaaaggttttttcttgctgctggtacgtataatgataacacgaggcctcgattttttgcgcgtattcggtgcataaatcagtatgttccttaaaaaacatatgtttttgaagcgttctaaaaaacatcatttggatgatatcacgcatttctaaaataatatagggttctaatcttttggaatctttcataactagatcggtggtaatattcttagtcatacaatttattaaaaatggcttaatatattgtaaatattttttaggcgtgtcagcctgtaaaaaacattcttgttcaatcttatttgtaaggatagtattttgcaaatgcttatttagcaaaaatacgatagaatcgcgggctatatgcattttcatataaatttttttttaaaatttaatacaaaaaaaagaagtatactctccttcttctagtctggttagttcgttggttgcctcaacatggagactcagaagttgatttccatggttaaagaagccttggaaaaatatcaataccctcttactgctaaaaatattaaagtagtgatacaaaaagagtacaatgttgtcttacctacaggttctataaatagcatactgtacagtaactcagaactttttgaaaagattgataagacaaatactatttatcccccgctttggatacggaaaactaactaaaaccagtagtacatttaaggatagtttaagcagtaaataacatagttaagcagtaaataacatagttaagcaataaataacaagtatatataggaatatatgatatataggaatatataggtatatagaaatatatagaaaaagctaagcttaatactaattcagctttttttttaactaaaacttgaatacatgcgaagtagcggacatatacatactaaaataaaccacacatttactttcttcttgaacatgaaacctttttttcttctgttgttggtatataaacaataggactgtttgctgaggttgtatgatcttctacaactgctgtctcaggatgacgatgtttttttaaactaaaagtgtaggatggaatgagtggaatatagttatggctcgacttatcctgtttcgtacaggaatattttttacaaatagaacgcaacaagcatatgaataaaaatagaaatgatatacaggagcataaaatagatatgaacactaaggggtagcagcttttataacgttccgtatttttcttagctatcaattgatttactgtaatatttatctcgggaaactttgttctacaatattttgtttggtattccagaaactcatgtcctggtttattcccgcagcttagaaaatgatacaaatatgtgttattgttactaaaactaattcttcttaagaaaaactgcggaagacgctttaggtacgtctgttcctgtttcagtaggaagtagtataagggacaatttctttttccacacattagattattgtaatataggtaggttggggtgttggagcgaataagttttctgagtatgttataatctatgacttgtaaatcgttataccttaggtccaaaaacttgagttctttaccaaagccacctgcaatttcagaaatatttttcatcccgcagcggatgatgcggatgtcctgaaacgtctttaaaatacttgtattgtagtgaatacttatgttatttttttgtaaataatctatgtcatgacaagtgcatgaaatgccagcagcattgcttggtatagtattatatgcaggaagaattatactactattgagaatagtcacattgtacttataccatgtattattttctgatataaagtatttgcaggtgacctgtggtttaatcctacctgttaagccacttcctaaaaaaacaaaaaatatgaaaacccttagcatcctgtatatactattaaaaatttataaaattttctgtttaaatttcatttagacaaaaaaaataatatatatacatcagcaagaaattatatacagattatataattttctgatttttttttgccataataagcatcattatatgcattaaaatgtcagtactaaacactaaattctaagcattaaattctaagcattaaattctaagcctaaattctatgcactaaactgtaagcactaaattctaagcactaaactgtaagcactaaaatctaagtaactaaaatcaacactaaaatgtaaagcattactcatcatccatctcttcatcctcctcttcttcatcatcatcataggttaagatatatgtgtcatcctccatttcttcacattcatcttcataagcatcactgggtattggtggaacattggatgcagcatttttaaaatattctatgtcttctggtgaacactcatctaatgattttttgacagtctttttaacttccatgggatatgattccaaatcctctttatatacatatttacggtagcttttagctgcatccacatttgctggagaatctggatttggctcattgagcaatgaaattacactaagaagaatggtatcaatcttttgagccggagaccaagtcattccctgttcttcagcattgtctccgtgtaagatagagatacatagtcttccatcagggtaaatattaggatgccacatttcagaggtgaatgttaatttgggtggtgcatatgggtattctggaggaaaggcgacttttgccttgaataagcctccctcataaagagtgtcaggtgggccccttaagatcacatcccattcagttatattattctcattcactgaaattttgaaattctcagaggggttctctatcaggtgtctgtactctgctattaaaaacctggaaaccatggttatttaatattaattaaattccctggtttattccttaaaagtagatgaacctcttttgttttttattgggttcatttttactaaatttatgaactggaaaaatctttaacggcataatcatcaaatgcgaagggggatccgtataaaatcctagcttgccggtaatggctattaagttaaatttagtacctgtaacagtaatatttaaaaagccctgatcattaactttccacattaaaagattattatattcgaatgtttgtccaatatggatgtctttgtcaccagatattacatttgtattggttgttagtggctgaagcttggcacaatcaaaaataagcccgttaacactaagatatagaggagtgggttgatctattttctcatagcttaatactccatttttccacgtaatagcttgataattatccgcagcaatgagttgaaattttataaatagtacaggggttttagttgtcgttatacatttaaagggtgttttataaaaataaaaaataataattgttaaaagtatgataataatcgccaaaataatttcatacattttttataagaattatacatagtatggtatttaaaatattagctaaatttaaaaaaacttcatgatttttaaagcagggaaaaaggggattaggttgaataaaaaaggtaagcacttgtctatatgttttttttataatgttgccttgagtcgcatttttaactggctggggagtatcagagtggaatatcactgtagtaggtctataaggtcttgttaaaatatcggtcatagtgtcatttagggtcgacctgatagctcgatataaagttataggggataaaccatcagatacagtcttatctgtgctaaaaggtgtatcgtcctctttatcactaataatattaggaatggcagacgttaaataattactacttgttgttgtaggcaaaatagttgtactagtattattagaaatggctgttagtaaataattgctacttgttgtaggtgaaatagttgtactagtattattagaaatggctgtcgttaaataattgctacctattacaagtaaactaatgctaactacatttttaacctcaataaacctaaaaagccatactaaatacctaaacaacatcctgttataatatgagcagaaaaaaaaaataagtataattagggaattattcttattcgcttactattaagaataattcagaatcttatttagttagaaactatcataaagtgaataggactcatcgtcggatgaagattccgtttcagagatagtttctttttctttctcagaataatctgttcctacaatagaatcggtgtcatcctcagaaagagaagtatttaaatatggactatctatagcaatatcctcttctatctcgcaatcctcctcctccatttccatagtgtgtaggagaatatttttatcatcatgttcacttctttttttgttgaaagatgaaccgtcctcaatgcggttcatgttaagttccttcatcttatgaataatttccgtaatccgtgatgtttttgacatgtaagatggttttaaggttatatccacaataacaggagaatctgtatcattttcatttgataaactttgatctttgatttcttcgtctaaaattcttgtctttttttgggtactagatgaaatcgaggaattcatactctgaaacgatatatcaaggggagctggacgcttttttccaattaaaccgtttttcgagatactatgtttagatgaatgatctttagccaagctgtccttggatatactatagttagatattttacctttaaataatattcttctatacaagttattcttaggtaaagaattagtatggattcctatatttttatctgaaggagtgtccatatcggagaacgtcctcttacgaatattttgaccacgagccatttcatccactataggcagtattctggctggctatggttctttgctgtgacaattctatgaaatttgattgcaaatcaatttttagttttaaatatattggtacctaggacaaagaaagtatatatagccaataattattccactaaattgatttccatactgatgggtatggagccatgttgtctctgcagacaatcgcgaaaatggccgtagcaacaaacacctactccaagtatcactatccaatactgaaggtctttgggctgtggtggaaaaacagcacgctaaatggccctattaaaatatgtaaccattgcaacaacataatggtaggagaatatcctatgtgttacaatcatggaatgagcctggatatagctttgattcgggcggtaaaagagcgcaatatatccttagtccagcttttcaccgaatgggggggaaatattgactatggggcactttgtgctaacactccatctatgcaaagattatgtaaaagtttgggagccaaaccaccaaagggccgaatgtatatggatgctcttatacatctttcagataccttgaatgataatgatctgattagggggtatgagatttttgatgataatagcgtgttggattgtgtcaatctcatacgactcaaaataatgcttaccttgaaggcccgtatacctctcatggaacaactagaccaaattgccttaaaacaacttctgcagcgatactggtatgccatggctgtacaacacaacttaacaacagctatccactattttgataatcatattcctaatataaagccatttagtctgcgctgtgctttgtattttaatgatccctttaaaatccatgatgcttgcaggactgtaaatatggatcctaatgagatgatgaacattgcttgtcaacaggatttaaactttcaaagcatttactatagttatattttaggggctgatattaatcaggctatgctaatgtctttaaagtatggtaatctttctaatatgtggttttgcatagatttgggggcggatgcctttaaagaggcaggggcgcttgctgggaaaaaaaaaaaaagagtgttacagcacatattaggtcttaatatctttaagcgggagttgattcccccctgtaaagatcctgatccttatcaaatccaaattctgttaaaaaactacattctaaaaaatgtctcaactgtttttacttattattgccagtagccattgtttatatcagaatataacccatttgtttatctttttttgtagggaccattaaagtccgacgcaaaaaaagtttaatcttttatcagatacctaaaacgttctataagggagtctatgagatggatcatattttgatggtcatagtaagaagcaagctttttggcgaaaacgacggagttaaagaatttaacccgctcatgtttggataggacttttaacagcgagccaaaacagtatttaaaaatttggcaatagtttttttgggatgcaataaacaaacacttgatcagtgcccgcttcactttctgatcagacatgtttgccgcataacaggcctttttaaacttagtaatataattatgttccgcaagcaccattaacaagggaacgatgggaagctgcttttctcggtgaaattcacgcaaatattcgatggccaccgcttggacgactgtgtaatttactaagttagaaataatagcttttatggttgtaaaaatatacataggattttctttttctgtatacagtttgaaaagcttatgattgcgtgaaatgatggccatttttaatacaagatggtatagtgtatctttaggtaaaaatgccttgcaagccgcgatgatgtcgatgttgtctccatgaacagcgatagaaactaatgtttccaatctaaatgtttttatctgcattaatagaggaatgcagtcaatgttattatacttaataatactgtaatacaccgaatcaatgaccgtcatctgagaatcaagctgacttattagtaaatttaacgtttttttggaggcatgacctttgattgcggcactaagtgcacacagtatagcaaaattgttaaatacgttttgatttaggagaaggagtaatattttccttcggttatagtacgcagcatctgtgatgattatttgccgataaatgttaaaatgcgttaacagctttttaaaaaaacggaagtaatttttttggatcgctgtttgcatcatcgaaataatgagataatcagggtatataatgggtaggtcacatgctacctctaacaaagaatagtcgcccaatctaaaggctgtgttgaaaagcgtactatcatcatacgtatcgagtacccccgctgttacaaaccaagcgataagatgaatgtgccgttccttgcaagctatcgcaaatagggtgtttcctatggaatgtcgaataatgtactccctatttttttccaaaatgtttggaaaattgtatagcgttgcggcatacagtagacactccattctggcgttataatttttacttttacatatgaataggtggaagaactcgaataattcttgagaacttgttaaatgcataatatggtgatattttggtgtcgttaaatgatatgagaaaatgcattctaatacatcttttcggttatgctttagcgcctgagctaaggcatattcaggctcgacccataggactagtgtttttataattgtgatattcgcctgctttgccagggcatactttaagacgctccggttggaaaaaattttgttatgaagatggacaacagtgtccatttttatgacgggaccattccagtatagtcctaaatgctgtagcagatcttttgttagttgtgaagcgttctcgggagtcatataaatatgttgcagggcttttttctgtaaggagaacatttcgtcgtaatcgtacaaaaaaaaattaaaatttgggcatggatgattcaaacataacaaaatcaggattttataacaggttgcatgaacttatacatatatgcaagtaaatgagatattatctatcataacgaatcaagggatatttgtatatatcaggagtttctgaaagaaagatatgaagagtatcatagtagtatccatcaatcacaatgcaacttcctttaagacataatttagtaaactcagcactcccatcttctggatgctttacaactaacattaaaaactcctcagttatattgtctgtaataaaataagatcctcctggagccatgtgtagcatgtctcttattcctacaaaatcttttttgggatggtaaaaactcagcagtttcaaactcttttttagttttttttcctggtatttaagccatttgttataaaacagttttcttatgaaaatgcatttgaaaatattgggaatgtttaaccatgcttcttccgagcacatctccagatacttactttctttgttccccatgtctaatttattgctcactaagttagtaatgaataacctatctatcttaataacctatctatcttataatctatcttaataacctaatgataacctatttataattagctaatgctgccggcatttcatgcacgatctaaacagttaattcctactaagcaatctactattacatatatagattcacttttttatatttgtaaatcatgataaaataattacttatttttattgtaaattagtgggtatttgtaaaaatcctcaaatgttttaagatagttttctagagagtagtaatctttaccattaatatacaatgctttccctttaaattccagttttgctatgtttagtgagccgttttccagatctttttgaacaataaatatattttcattggttgcatcgtccgtaagcagaaaggtaccattaggcatgctaaataacattcgttctatttcatggtcggatttttgagaataaaaaaaacctaattttttaatccgcgttaactcttgtttatcaatctttccagactgttttatgtatactttattgcaaatcttagaatcctctatggtttcattatacttattttgcttatcctctattgacatgaccgtatttggtaggtaacttctgataagacggttccccatggttttaggtgtagatttttaattcagttgtgtacttttactatgaggctaaaataaatagttaaatatagaagttgaacctaaaaaataaaaagatttatggtttatagcggtatagaggacgataaaaggtatccgggtagtctcctatgatatcgtcaattttggtataataacacttgttatggtagtattgtccaaaccgagtatgtatgcgccggtgaagcgtccgcccgctaatggtacagttccaggttaagacaatcatatcacacccaaaaagagaggaaacagcataggtgcccaaaggttcattatataacatacgccgcatatattttagttttttttctccatggtaataatcacagtttttcatgtcccgcttaataggatgattccccatgtatgataatatataaaaaatttagtttttagctttttcaaaaaattgggtgctcgaaactaaattttccttatcacagtgtttggagaaagtgtatttaaagatatatcttctaataagactgcataaaaaaaattcttacccttatttttataatgttgtagttcataacagcgtttgaagacatcagagggtggcccaggttttataaaaatatcctttaggatttataacgatacaagggtctataaaatatatgcgggtataatcttataaaatcatcgattttttcataatattctccgtttatacaataaagatcataacagatattgatgcgtagatgcattattcgcgtgttcgttgggcagctaaaggatatcacaacgtagttttttttaagaaaagacgaaactacataagtccctaagggttcattgaatagtaaacgccatatttgttttaaattttgttgttcaccatagtagtattcgcactttttcaagtcttttttaataagcctattccccatgtatgcttataaataaaaatttagaaatgctatattatttgttgatgaatgatgaacacgtcttatatgttgatatattactttaaaaacatttgtattttcaacagatgcgatctattcttattaagaatgatgccgtctttattttaaaccttggtttaaaatttaaagaagtatttataaactataatcatggaaaccttttcagtaactgcctctgcgaaaagtgacgatgctgtttgtaagtatctagaagaaccaatagatgaaaataaaaattctagaaacatattaagaaatgaacatggtaaaaaaaaattaaatgaggctctgaatcgacatataattgcttataatccagtagttgattggtgtaataactattcaacatattcatctcagtatttcgatgaatataaaatttatatacatagcgatcttatggatggacgacctcgtccaaaaaaaacatggtgtgtcatcatgtaatgttcgtttgtttcatataaacacaaaaatattctaggagatattaatatactacctattgaattcaatatattaaagtacatttctggctatttccattacggtcgtattattactatttttaagagctggatgtggatttgtgtaacaatagcattttccctctcctcctagagacatctcatcaaattcccatcctatgcaacctttatgttgtaaacataatgattgacagtaatcagcttcccttccccaactagtccaaatcttaccaagatctatacgtgtttttccaaaaggagattgaagatcagcagtagtggcattaaacctataaaaaccaggtgtataatcacatgaacggatggtaggatctaatttaatatcttttatatcttgttttacagcttctagacaacttttatcagtacatgttccacgtacacagtggtgtcctttatccttacaatccgtatctgtcttacatttttttttcggcagtttatgttttatatggtaaaaacccagtattaaaataatcacaagaataattcctataagtacttgaatagcaggataaaacattttaatattaaatatattttttaattaaatgaatagatttaatccaagtagtattaaaattttttagaaatagtgttctacaaataatgaaatgaatagcccaaaaaaaataaggtgtacattaatgtaatatattgttaggctaagtaaatttaatatttaaagtacttggaaaaatattttttaacatatgctgtctaggaatttttttagacatttaaaaccatatagttactttatttattacacttaacttgaaaagaattattgcctaaaatattaatagatgaagtaatattgtgtaattgagtccataacatgggtgggaaacaaaaatctcgtaatatgaaaaataaacatcctaaaaagagtgcaattgttataagtttatgtaactttattttaaagtaagaatataaaaatatgagtacaagaggaataggggccattactaatattggctccaacatcctgttgtctacaaaaaaaaatattttttttggcaaaaaaaaatccatggaaggatattaatacacataattgtttgacatcatattggtatacttaccaaatagtaatatacaaccatcctaatattcactttatgaaatgaccccccccccccaacctatacagtaaaatagtataggttttaataaagaaaaaagatattatgtagtttttatttttgtatagtgcgtgaatgcaaaataaaatcccaaattttaaccttttctttttttttctatacaggatgttagaaatagtattggcaacgctgctaggcgacctgcagcggctccgggttcttacccctcagcagcgggcggttgccttctttcgagccaatactaaggagctagaggacttcttacgctcagatgggcaatctgaggagatactgtctggccccctccttaaccgtctactagaaccctcatgccctcttgatattttaaccggatatcacctatttcgtcagaatcccaaggcaggtcagttgcgcggccttgaggtcaagatgcttgaacggttatacgatgctaatatttacaatatattgtctcggctgcgacctgaaaaagtccgcaacaaggctattgagctatactgggttttccgagctatccatatttgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccagataagtcttgagagcaacaaccaggtagggatttgctatgcttacttgatgggaggcagcctcaagggactagtctccgccccactgcgtaaacgtctgcgcgccaaactacgctcgcagcgcaaaaagaaggacgttctttcaccccacgacttcttactgctgctccagtagctttttttgccgcaggagcaccgtggataggagctcctccacgctcgcgatccggcgctggaagcggaaccgatcgaccgccacctgctcccagggacccttgcgctcgatgtcgtcggcttcccatacctcgacggctgcagcaaagcggacgtgcttcgtgtcattcgtccgttttttgcgccgccgcccccccattattcctgtaagattagtgtttaatacctataataacataattttaagatttaatataccaaaacttaaactatttttgtatagtaactattagcatgtctacacatgattgtttttcaaaagagaaaccagttgatatgaacgatatatctgagaaatcatctgtcgtggataatgcacccgagaaaccagctggagcgaatcatatacctgagaagtcggcccgcgaaatgacatcatcagaatggattgctgaatattggaaaggtataaaacgtggaaatgacgtgccatgttgttgtccaagaaaaatgaccagtgcagacaaaaagttttcagtatttggtaagggatccctaatacgctccatccagaagaataattaaaaaaaatattttttttggtaagtttataaactatatagttaaatatggtaaaaaaaatcacataataattaaactgaacgtgttagaattaatatttttttataatcggatataatatccattaaatcaataaatgatagtgttgctaccacactaaacaataacaaacagaaacgcatgatacctttcctcatgatttataatagcgagttatctaaagatttttttgaaaaaaatattaaattttagttgatatatttttttagttacaacattgctttagaaaaaaaacctaattactacatagcaaataaagcgagcgtgttgttacaaacaacatttttttgcgcctggatactcttatatatgagaactataatatgccatattaatcctattaccaacaatgccaaaaatagtatgtaggcaatgacatactttaaatacaaaatttccatggttatctctaaaaatcttgaaaaaatatttaattttagatcggtcacctacggtagtaatactaattttaataattgatgactaaaatcatattataatgccgtgcaaaaaataattatttttcggttaaaggatacctttaaataaaaaacgatgccatccactctacaagtgcttgctaaaaaggtgttggccttaggggaacataaagaaaatgaacatatatctagagaatattattatcatatattaaagtgttgcggtttatggtggcatgaggctccgattatactttgttttgatgggagtgagcaaatgatgataaagactccaatctttgaagaaggcatattacttaatactgcattaatgaaagctgtacaggagaacaattatgaattaataaacttgttcactgaatggggagcaaacatcaattatggattaatttccattaatactgagcatgcccgggacctatgtcgaaaattaggagctaaagaaatgcttgaaagaaatgaagttatacaaattgtatttaaaacattagatgatatcaccagtagtaatataattttatgtcatgaattatttaccaacaatcctcttttagagaatgtaaatatgggggaaatgaggatgataattcattggaggatgaaaaatttaacgaacctattattaaataatgactctattagtgaaatattaactaaattctggtatggtatagcagtaaaatataatcttaaggatgcaatccaatatttttaccagagattcatggacttcaacgagtggcgagtaacatgtgctctttcttttaataatgtgaatgatcttcataagatgtatataacagagaaggttcatatgaataatgacgaaatgatgaatctagcctgcagcattcaagacagaaatttatcaaccatttactattgttttctattgggggctaacatcaatcaagcaatgttgacctcagtattaaattataatatttttaacttattcttttgtatagacttaggggctgatgcctttgaagagggtaagaccctggcgaaacaaaaggggtataatgaaatagtggaaatcttatcattagatatcatttatagtccaaatactgacttctcatcaaaaatagaacctgaacatattagttctttgttaaaaaacttttatccaaaaaatctgttcgcttttgatcgttgcaaccccggtttatattattcttagaggaccgctacaaaaattatttttttttcttgatcaaagctccaaaataattattagattaaagtcgcctatagcagctgcccactccaaaaaaagtattttatagtacaaaaaacacgaaaaataatttgcggccggcggcaaactatttgttgttgtccaaaacttaatgtttttttaatatttttaaatgcaaccatggattgttggactatcagggagaagaactatagctacatcatattgtcaatactggtaatactattaatatggtatcttatacttaactattgtcgatcgaaaaaaaaatgcagttacaaacaacatgccgccaccagcatacacggtgtcaagtagctgttctcaataatagggttgatcgacgctcttcgtaataatatgttgattggcgcatcataaaatgctgaggaactatccgttaatatatgttgattgacgcccacttatgaatggaagtaatgattttgtataaaatacgggtttgtgagggccttatttttttcttattagaacaaagcgtgtatttttaaggcctatagcaagagtatgtttaattaacacctacaacagtaatatttaaggccagtaaaataatgtttaattaaggcctgaccactaaaacttaaacgattttgtaaaaaaatatgcctactccactttctctacaggctcttgctaaaaaagtactggccacacagcacatatctaaagatcacctttattttgaaatattgtggtttatggtggcattctttgacgcctacagtctttaacgcctgcagtaataattgatatctccagcgctaataataattgatacccacagcggtaataataattgatacccacagcggtaatgtcgtccatctggccgccccaaaaaaaagtatttacggtggggtttattaccggcggcgtaacaccagttatggttagttttgtctggcccgccgcccagccgcaaaaaaaaatcaattacagccgcaaaaaaaaatatttccggccgcggagtttctacaaaaaaaatgttagcttttaattgttgtagaccaggttcttagggaaggttcaactcctttttctcgaataaaaaatcttaccctccaaaacttaatgtttttttttgggatttttaggtgcaacatggatcattggactaccaagaggcaggtctatatctacttatgtttctcattgatgacaatagcattaatttgctatcttatacatatctgttgtcatacgaaaaaaaacgttgttactaacgcgctgccttcaaacaacatggcgctaataccatacacgccttcaaacaacacggtgccaccaccatacacgatatcaggtagctgtccccaataatgtgttgattcacgcccatagaatgctgtgattgattgatgcccataaaatggtgtgattgattgacgcccataaaatggtgtgattgattcacgcccatagaatgctgtgattgattgatgcccataaaatggtgcacttagatttttgtatatttcttgatcaagcgaaaaacattaaatagtatacagctaaactacttaacaggatatatatagtgggcctcctgccaataagatgtataaagagcccacataagatggttatagtatagcactttaacatgaatatcaggaatgccaatagcatgtagataaaacaccaaacatcacagatatacatggctgcttttttttgtgactggcgattttttacgaaaaaacattaaaatttagctacgcctacagcagtaataatgtttaacgcctacagcagtagtaataattttaatgtttaacgcctacagcagtagtaataattttaatctttgacgcctacaacagtagtaataattttaatgtttaacgcctatagcagtagtaataagtttaatgtttaacgcctacagcagtagtaataattttaatctttgacgcctacagcagtagtaataattttaatgtttaacgcctacaacagtagtaataattttaatgtttaacgcctacagcagtagtaataattttaatgtttaacgcctacagcagtagtaataattttaatctttgacgcctacaacagtagtaataattttaatgtttaacgcctacaacagtagtaataattttaatgtttaacgcctacagcagtagtaataattttaatgtttaacgcctacagcagtagtaataattttaatctttgacgcctacaacagtagtaataattttaatgtttaacgcctatagcagtagtaataagtttaatgtttaacgcctacagcagtagtaataattttaatctttgacgcctacagcagtagtaataattttaatgtttaacgcctacaacagtagtaataattttaatgtttaacgcctatagcagtagtaataagtttaatgtttaacgcctacagcagtaataataattttaatctttaacgcctacagcagtacaaattattttaatacctatcgcccaccggtatgcaccgcgaaatctagtgaaactacggtacacaaaatactttccggcgaaaaaataattattccggcccgccaaattctgcatcttgtgaaactacaaaatgctttccggcgaaaaaataattattccgccacgggcagacaactgaaaaaaaaatcaatcgccactgcaaaaatatttccgcgcaaaaaaaataattattccggcgcgcaaaaaaat

(19) United States
(12) Patent Application Publication
(10) Pub. No.: US 2015/0165018 A1
(43) Pub. Date: Jun. 18, 2015

(54) CD2 DEFICIENT AFRICAN SWINE FEVER VIRUS AS LIVE ATTENUATED OR SUBSEQUENTLY INACTIVATED VACCINE AGAINST AFRICAN SWINE FEVER IN MAMMALS

(71) Applicants: Boehringer Ingelheim Vetmedica GmbH, Ingelheim am Rhein (DE); Consejo Superior De Investigaciones Cientificas, CSIC, Madrid (ES)

(72) Inventors: Fernando RODRIGUEZ, Montgat-Barcelona (ES); Maria Luisa SALAS, Madrid (ES)

(21) Appl. No.: 14/574,665

(22) Filed: Dec. 18, 2014

(30) Foreign Application Priority Data
Dec. 18, 2013 (EP) 13197957.7
May 9, 2014 (EP) 14167771.6

(51) Int. Cl. A61K 39/12; C12N 7/00; A61K 45/06

(52) U.S. Cl. CPC A61K 39/12; A61K 45/06; C12N 7/00; A61K 2039/5254

(57) ABSTRACT

The present invention is directed to a preferably live attenuated or subsequently inactivated African swine fever virus (ASFV), comprising a non-functional genomic CD2 gene, wherein such ASFV is non deficient in its replication, as well as to corresponding compositions or immunogenic compositions or vaccines, methods of production and uses for treating and/or preventing African swine fever in mammals, preferably of the family Suidae, for instance pigs, more preferably domestic pigs (Sus scrofa domesticus), wild pigs (Sus scrofa scrofa), warthogs (Potamochoerus porcus), bushpigs (Potamochoerus larvatus), giant forest hogs (Hylochoerus meinertzhageni) as well as feral pigs.

### SEQUENCE LISTING

This application contains a sequence listing in accordance with 37 C.F.R. 1.821-1.825. The sequence listing accompanying this application is hereby incorporated by reference in its entirety.

### FIELD OF THE INVENTION

The invention relates to the field of medicine, in particular to the field of veterinary medicine. The invention relates to a preferably live attenuated or subsequently inactivated African swine fever virus (ASFV), comprising a non-functional genomic CD2 gene, wherein such ASFV is non deficient in its replication, as well as to corresponding compositions, immunogenic compositions or vaccines, methods of production and uses for treating and/or preventing African swine fever in mammals, preferably of the family Suidae, for instance pigs.

### BACKGROUND INFORMATION

African swine fever (ASF) is a highly infectious disease affecting domestic pigs, included in the former list A of the world animal health organization. In spite of its eradication from Continental Europe in the mid-nineties, ASF virus (ASFV), the etiological agent of ASF, remained endemic in Sardinia and in many Sub-Saharan countries, where it causes tremendous economical losses. The recent reintroduction of the virus in Georgia from Eastern Africa and its spreading toward Russian counties has opened new concerns about the risk of ASFV re-entrance to Europe and Asian countries, including China, the major swine producer and consumer in the world. The situation becomes aggravated by the fact that there is no vaccine available against ASFV, therefore limiting the control measures to an efficient and rapid diagnosis of the disease and culling of the infected animals; a measure totally out of reach for the poorest countries affected by the disease. Work made in the past clearly demonstrated that inactivated preparations of ASFV fail to confer protection against the viral challenge (Stone and Hess, 1967; Forman et al., 1982; Mebus 1988). In clear contrast, immunization of pigs with attenuated isolates of ASFV have demonstrated to induce very solid protection against the homologous viral challenge (Ruiz Gonzalvo et al., 1983; Wardley et al., 1985). Despite safety issues made impossible the application of attenuated viruses as vaccines (Sanchez Botija, 1963), they have provided with the most useful data existing today about immune parameters involved in protection that include:
i. neutralizing antibodies (Onisk et al., 1994) and/or antibodies capable of inhibiting ASFV in vitro infections (Ruiz Gonzalvo et al., 1986)
ii. specific CD8+ T-cell responses (Oura et al., 2006).

Neither antibodies, nor T-cells seemed to be able by themselves to confer sterile protection, clearly indicating an ideal vaccine against ASFV should be able to confer both kinds of immune responses.

Deletion of specific virulence genes by homologous recombination, led to attenuated ASF viruses, although experimental evidences of their use as live attenuated vaccines are limited to only few literature references at its best; all of them published more than 10 years ago (Borca et al., 1998; Moore et al., 1998; Lewis et al., 2000; Neilan et al., 2002).

The high complexity of ASFV (comprising more than 150 antigens encoded) renders the task of selecting the optimal antigens to be included in a subunit vaccine difficult. Work done in the mid-nineties described the protective potential of three ASFV structural proteins: p54, p30 and hemagglutinin (HA), when expressed in a baculovirus system and administered without further purification together with Freund's adjuvant (Ruiz-Gonzalvo et al., 1996; Gómez-Puertas et al., 1998). Although protection was associated with the induction of neutralizing and inhibitory antibodies, respectively, the induction of cellular responses should not be ruled-out. More recently, these studies have been extended to the field of DNA immunization. Thus, DNA immunization with a plasmid encoding p54, p30 and the extracellular domain of the Hemagglutinin (sHA), fused to ubiquitin, conferred partial protection against lethal challenge in the absence of antibodies. Protection correlated with the expansion of CD8+ T-cells, specifically recognized two 9-mer peptides within the HA (Argilaguet et al., 2012).

In summary, disadvantages of the currently available vaccines against African swine fever in mammals include: lack of efficacy (inactivated vaccines), lack of safety (natural live attenuated vaccines that can revert to virulent) or lack of solid experimental evidences (recombinant vaccines including subunit vaccines and recombinant deficient live attenuated viruses).

Finally, one of the most promising strategies based on inducible viruses, has not been optimized for use in vivo: WO 2012/107164 relates to vaccines against ASFV based on replication deficient recombinant viruses, preferably based on virulent ASFV isolate BA71 in which the expression of essential genes such as pp220, pp62 or pB438L is inducible in vitro and therefore, repressed in vivo. So far, only one replication deficient ASFV has been used and tested as a candidate vaccine: BA71.v220i.TK⁻. This recombinant isolate was based in the inducible expression of the gene coding for the ASFV polyprotein pp220. For this purpose, the Lac I repressor, together with β-glucuronidase marker gene, was introduced in the TK locus of the virus genome. Under in vitro non permissive conditions, the resulting recombinant isolate, BA71.v220i.TK⁻, leads to the assembly of non-infectious icosahedral core-less particles capable of exiting the infected Vero cells (Andrés et al., 2002). However, in vivo immunization with BA71.v220i.TK⁻ did not confer protection against a challenge with the homologous BA71 probably because, although the TK gene is dispensable for growth in tissue culture cells, it is essential for virus replication in porcine macrophages and in the infected animal (Moore et al., 1998). Lack of protection correlated with the failure of BA71.v220i.TK⁻ to induce humoral or cellular responses in vivo.

Further prior art is as follows:

Borca M V and co-workers (J Virol 1998, 72 (4): 2881-2889) relate to the deletion of a CD2-like gene, 8-DR, from ASFV, i.e. pathogenic African isolate Malawi Li1-20/1, which affects viral infection in domestic swine. However, the authors report that despite the CD2 deletion the Malawi mutant isolate is still highly pathogenic (page 2886, left-hand column, second last paragraph, as well as page 2884, Table 1) and is therefore not suitable for vaccination.

Kay-Jackson P C (J General Virol 2004, 85: 119-130) deals with the interaction of CD2 protein of ASFV with the actin-binding adaptor protein SH3P7.

Boinas F S et al. (J General Virol 2004, 85: 2177-2187) are directed to the characterization of pathogenic and non-pathogenic African swine fever virus isolates from Ornithodoros erraticus inhabiting pig premises in Portugal, among others ASFV isolates OURT88/1, OURT88/2, OURT88/3 and OURT88/4.

Chapman D A G and co-workers (J General Virol 2008, 89: 397-408) disclose a comparison of the genome sequences of non-pathogenic and pathogenic ASFV isolates, i.e. non-pathogenic ASFV isolate OURT88/3 from Portugal and highly pathogenic ASFV isolate Benin 97/1 from West Africa as well as tissue culture-adapted ASFV isolate BA71V. They show that ASFV isolate OURT88/3 has interruptions in open readings frames (ORFS) that encode a CD2-like (EP402R) and a C-type lectin (EP153R) protein (abstract as well as page 403, right-hand column, second last paragraph, and page 406, right-hand column, second paragraph from top).

King K et al. (Vaccine 2011, 29 (28): 4593-4600) describe the protection of European domestic pigs from virulent African isolates of ASFV by experimental immunization. King and co-workers showed that experimental immunization of pigs with the non-virulent OURT88/3 genotype I isolate from Portugal followed by the closely related virulent OURT88/1 genotype I isolate could confer protection against challenge with virulent isolates from Africa including genotype I Benin97/1 isolate and genotype X Uganda 1965 isolate. However, the authors reported that in their second experiment only 60% of the pigs survived the challenge with Benin 97/1 isolate (page 4594, right-hand column, last paragraph).

Abrams C C and Dixon L K (Virology 2012, 433(1): 142-148) deal with the sequential deletion of genes from the African swine fever virus genome exemplified on tissue-culture adapted non-pathogenic ASFV isolate BA71V using the cre/loxP recombination system. However, such totally non-pathogenic ASFV isolate cannot infect a host in vivo (even at high doses, such as 10⁷ plaque forming units) and cannot provoke a respective immune response.

Escribano J M and co-workers (Virus Research 2012, 173 (1): 101-109) relate to the antibody-mediated neutralization of ASFV, its myths and facts.

The objective underlying the present invention is therefore to provide a medication for preventing and/or treating African swine fever in mammals, which overcomes the problems of the prior art.

### SUMMARY OF THE INVENTION

In one aspect, the objective of the present invention has surprisingly been solved by providing an African swine fever virus (ASFV), preferably a non-naturally occurring recombinant ASFV, comprising a non-functional genomic CD2 gene, with the proviso that such ASFV is non deficient in its replication, wherein preferably such ASFV is a live attenuated ASFV or subsequently inactivated ASFV that was yielded from the live attenuated ASFV through subsequent physical and/or chemical inactivation.

Such physical inactivation is preferably achieved by subsequent treatment of the live attenuated ASFV with UV radiation, X-ray radiation, gamma-radiation, freeze-thawing and/or heating. Such chemically inactivation is preferably achieved by subsequent treatment of the live attenuated ASFV with one or more chemical inactivating agents, wherein more preferably such one or more chemical inactivating agents are selected from the group consisting of beta-propiolactone, glutaraldehyde, ethyleneimine, beta-ethyleneimine, binary ethyleneimine, acetylethyleneimine, ozone and/or formaldehyde.

In the context of the present invention, the term “non-functional genomic CD2 gene” refers to a modified CD2 gene, such as EP402R, located in the genome of an ASFV, preferably a non-naturally occurring recombinant ASFV, wherein such modification of such ASFV CD2 gene results in no ASFV CD2 gene product at all or a biologically not functional ASFV CD2 gene product as compared to a non-modified functional ASFV CD2 gene. Including but without being limited to that such modification can be for instance a full or partial deletion of the genomic ASFV CD2 gene and/or the modification of one or more nucleotides controlling and/or encoding the corresponding ASFV CD2 gene product and/or disruption of the ASFV CD2 open reading frame (ORF), for instance by inserting one or more nucleotides into that ASFV CD2 ORF, and/or any other currently known or conceivable method of inactivating or knocking-out functional expression of such ASFV CD2 gene. By means of such ASFV CD2 gene inactivation or knock-out preferably a live attenuated or subsequently inactivated ASFV can be generated.

In the context of the present invention, the term “non deficient in its replication” refers to an ASFV, preferably a non-naturally occurring recombinant ASFV, which is able to replicate in vitro and/or in vivo and/or is capable of producing viral progeny although such replication and/or viral progeny production may also occur at somewhat reduced levels, for instance below the detection limit of state-of-the-art analysis methods and/or apparatuses. Therefore, it can be the case that such ASFV is non deficient in its replication in in vitro, e.g. in a cell culture, for instance cultured macrophages, although in vivo in a mammal such ASFV is at least severely impaired in its replication, e.g. resulting in a replication and/or viral progeny production below detection limits.

In another aspect, the objective of the present invention has surprisingly been solved by providing a method for the generation of a non-functional ASFV CD2 gene in an ASFV genome, comprising the steps of:
a. introducing one or more full or partial deletions into the ASFV CD2 gene and/or modifying one or more nucleotides controlling and/or encoding the corresponding ASFV CD2 gene product and/or disrupting the ASFV CD2 open reading frame (ORF) thereby rendering the ASFV CD2 non-functional, preferably by introducing Lac I repressor together with β-glucuronidase marker gene into the ASFV CD2 locus, leading to the almost complete deletion of ASFV CD2 gene thereby rendering it non-functional in vitro and in vivo.

In yet another aspect, the objective of the present invention has surprisingly been solved by providing a method for the production of a non-naturally occurring recombinant ASFV, comprising a non-functional genomic CD2 gene, with the proviso that such ASFV is non deficient in its replication, as described and/or defined herein, comprising the steps of:
a. preparing a non-naturally occurring recombinant ASFV, comprising a non-functional genomic CD2 gene, according to above method for the generation of a non-functional ASFV CD2 gene in an ASFV genome;
b. infecting primary porcine macrophages that do not inactivate the virus and/or a cell line susceptible to infection by ASFV that does not inactivate the virus, preferably COS-7 cells, with the ASFV of step (a) in vitro;
c. isolating the ASFV from the cells of step (b) and/or purifying it, preferably by collecting the culture medium containing the extracellular ASFV, centrifuging it first at low speed to remove cellular debris and then at high speed to sediment the virus and resuspending it in PBS, wherein optionally this resuspended virus is purified by centrifugation on a 25% saccharose cushion in PBS before finally resuspending the virus in PBS;
d. optionally, titrating the isolated and/or purified ASFV of step (c), preferably by the formation of lysis plaques [ASFV concentration is expressed as plaque forming units (pfu) per mL];
e. optionally, physically inactivating, preferably by treatment with UV radiation, X-ray radiation, gamma-radiation, freeze-thawing and/or heating, and/or chemically inactivating, preferably by treatment with one or more chemical inactivating agents, wherein more preferably such one or more chemical inactivating agents are selected from the group consisting of beta-propiolactone, glutaraldehyde, ethyleneimine, beta-ethyleneimine, binary ethyleneimine, acetylethyleneimine, ozone and/or formaldehyde, the live attenuated ASFV obtained from steps (c) or (d) thereby yielding one or more subsequently inactivated ASFV.

In yet another aspect, the objective of the present invention has surprisingly been solved by providing a non-naturally occurring recombinant ASFV obtainable by a method as described and/or defined herein.

In yet another aspect, the objective of the present invention has surprisingly been solved by providing a composition or immunogenic composition or vaccine comprising a therapeutically effective amount of one or more ASFV as described and/or defined herein, optionally additionally comprising one or more pharmaceutically acceptable excipients and/or one or more pharmaceutically acceptable carriers, wherein preferably such one or more pharmaceutically acceptable excipients and/or one or more pharmaceutically acceptable carriers are selected from the group consisting of: solvents, dispersion media, adjuvants, stabilizing agents, diluents, preservatives, antibacterial and antifungal agents, isotonic agents, adsorption delaying agents.

In the context of the present invention, the term “immunogenic composition” refers to a composition that is capable of eliciting a cellular and/or humoral immune response but does not necessarily confer full or partial immune protection against African swine fever in mammals. In other words, such immunogenic composition can lead to no immune protection at all. For the avoidance of doubt, however, such immunogenic composition may confer full or partial protection against African swine fever in mammals and this is also preferred. In contrast, a “vaccine” in the context of the present invention does confer full or partial, but at least partial immune protection against African swine fever in mammals.

In yet another aspect, the objective of the present invention has surprisingly been solved by providing the one or more ASFV as described and/or defined herein or the composition or immunogenic composition or vaccine as described and/or defined herein for use in a method of treating and/or preventing African swine fever in mammals, preferably of the family Suidae, for instance pigs, more preferably domestic pigs (Sus scrofa domesticus) and wild pigs (Sus scrofa scrofa) in Europe, warthogs (Potamochoerus porcus), bushpigs (Potamochoerus larvatus) and giant forest hogs (Hylochoerus meinertzhageni) in Africa, as well as feral pigs in the American regions (which are probably partially derived from European wild boar).

Corresponding methods of prevention and/or treatment of African swine fever in mammals, preferably of the family Suidae, for instance pigs, more preferably domestic pigs (Sus scrofa domesticus) and wild pigs (Sus scrofa scrofa) in Europe, warthogs (Potamochoerus porcus), bushpigs (Potamochoerus larvatus) and giant forest hogs (Hylochoerus meinertzhageni) in Africa, as well as feral pigs in the American regions (which are probably partially derived from European wild boar) in need thereof and uses for the preparation of a pharmaceutical composition/medicament for the prevention and/or treatment of African swine fever in mammals, preferably of the family Suidae, for instance pigs, more preferably domestic pigs (Sus scrofa domesticus) and wild pigs (Sus scrofa scrofa) in Europe, warthogs (Potamochoerus porcus), bushpigs (Potamochoerus larvatus) and giant forest hogs (Hylochoerus meinertzhageni) in Africa, as well as feral pigs in the American regions (which are probably partially derived from European wild boar), are also intended to be within the spirit of the present invention.

In yet another aspect, the objective of the present invention has surprisingly been solved by providing a method for eliciting a protective immune response in an animal, preferably of the family Suidae, for instance pigs, more preferably domestic pigs (Sus scrofa domesticus), wild pigs (Sus scrofa scrofa), warthogs (Potamochoerus porcus), bushpigs (Potamochoerus larvatus), giant forest hogs (Hylochoerus meinertzhageni) as well as feral pigs, administering to such animal the one or more ASFV as described and/or defined herein or the composition or immunogenic composition or vaccine as described and/or defined herein.

The preferred AFSV isolate (BA71.ΔCD2) according to the present invention is characterized by the following advantages: the viral CD2 gene is almost completely deleted. The only sequence remaining is a 36 base pair sequence at the end of the gene. This sequence is probably not included in any viral mRNA since the inserted cassette ends in a 10T transcription termination sequence. The deletion of CD2 does not affect viral replication in COS-7 cells in vitro. This characteristic, together with the fact that BA71.ΔCD2 can grow in COS-7 cells ensures the production of high titer stocks of the ASFV for vaccine purposes. It is important to notice that field ASFV isolates do grow exclusively in primary porcine macrophages, much more difficult and expensive to maintain and use to grow viruses for commercial purposes.

One additional advantages of this vaccine is its defective in Ornithodoros ticks, the non-mammal reservoir of ASFV since CD2 is a key virulence factor for ASFV replication in this invertebrate (Rowland et al., 2009). ASFV can infect Ornithodoros and remain asymptomatic for more than a year (Boinas et al., 2011), being a continuous source of virus for the environment and also an ideal in vivo vessel for recombination. The fact that CD2 deficient viruses will deficiently propagate in Ticks avoids any risk of reversion by recombination with circulating ASFV isolates. Furthermore, ASFV isolate BA71ΔCD2's, preferably BA71.ΔCD2's, CD2 deletion as illustrated supra is the result of targeted recombination, i.e. there is no risk of spontaneous reversion as in the case of, for instance, a frame-shift mutation, as for e.g. ASFV isolate OURT88/3.

attenuated isolates are limited to homologous protection only. The genome of virulent ASFV isolate BA71 has been completely sequenced (GenBank entry: KP055815; also referred to as SEQ ID NO: 4 in the herewith submitted sequence listing). From such sequencing it can be deduced that genes EP402R (CD2) and EP153R (C-type lectin) are fully functional and do not contain any deletions and/or frame-shift mutations.

In other words, above referred to ASFV isolate BA71ΔCD2, preferably BA71.ΔCD2, contains a fully functional EP153R gene encoding the C-type lectin protein. This is in contrast to ASFV isolate OURT88/3 which does contain multiple deletions and additions, such as interruptions in ORFs that encode the CD2-like protein (EP402R) and C-type lectin protein (EP153R).

Finally, the most important advantages of this vaccine come from the in vivo evidences herein presented. When used as a vaccine, it is safe, very immunogenic and capable of protecting against homologous and heterologous virus challenges. The last virtue renders this vaccine unique since natural

### DETAILED DESCRIPTION OF THE INVENTION

Before the embodiments of the present invention are described in further details it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA technology, protein chemistry and immunology, which are within the skill of the art. Such techniques are explained fully in the pertinent literature.

In the context of the present invention, the terms “protection against African swine fever”, “protective immunity”, “functional immunity” and similar phrases, means a response against African swine fever (virus) generated by administration of the one or more ASFV as described and/or defined herein or the composition or immunogenic composition or vaccine as described and/or defined herein, that results in fewer deleterious effects than would be expected in a non-immunized mammal that has been exposed to African swine fever (virus). That is, the severity of the deleterious effects of the infection is lessened in a vaccinated mammal. Infection may be reduced, slowed, or possibly fully prevented, in a vaccinated mammal Herein, where complete prevention of infection is meant, it is specifically stated. If complete prevention is not stated then the term includes partial prevention.

In contrast, in the context of the present invention, the term “immunoprotection” in connection with functional genomic ASFV CD2 gene and/or complementation by a functional non-genomic ASFV CD2 gene, refers to protection of an ASFV in a host from the host's cellular immune response or cellular and humoral immune response against such ASFV.

In the context of the present invention, the terms “reduction of the incidence and/or severity of clinical signs” or “reduction of clinical symptoms” mean, but are not limited to, reducing the number of infected mammals in a group, reducing or eliminating the number of mammals exhibiting clinical signs of infection, or reducing the severity of any clinical signs that are present in one or more mammals, in comparison to wild-type infection. For example, it should refer to any reduction of pathogen load, pathogen shedding, reduction in pathogen transmission, or reduction of any clinical sign symptomatic of African swine fever. Preferably these clinical signs are reduced in one or more mammals receiving the one or more ASFV as described and/or defined herein or the composition or immunogenic composition or vaccine as described and/or defined herein by at least 10% in comparison to subjects not receiving the one or more ASFV as described and/or defined herein or the composition or immunogenic composition or vaccine as described and/or defined herein and that become infected. More preferably clinical signs are reduced in mammals receiving one or more ASFV as described and/or defined herein or the composition or immunogenic composition or vaccine as described and/or defined herein by at least 20%, preferably by at least 30%, more preferably by at least 40%, and even more preferably by at least 50%.

In the context of the present invention, the term “increased protection” means, but is not limited to, a statistically significant reduction of one or more clinical symptoms which are associated with infection by a wild-type ASFV, in a vaccinated group of mammals versus a non-vaccinated control group of mammals. The term “statistically significant reduction of clinical symptoms” means, but is not limited to, that the frequency in the incidence of at least one clinical symptom in the vaccinated group of mammals is at least 10%, preferably 20%, more preferably 30%, even more preferably 50%, and even more preferably 70% lower than in the non-vaccinated control group after the challenge with the wild-type ASFV.

In the context of the present invention, the term “long-lasting protection” shall refer to improved efficacy that persists for at least 3 weeks, but more preferably at least 3 months, still more preferably at least 6 months. In the case of livestock, it is most preferred that the long lasting protection shall persist until the average age at which animals are marketed for meat.

In the context of the present invention, the term “immune response” or “immunological response” means, but is not limited to, the development of a cellular and/or antibody-mediated immune response to the one or more ASFV as described and/or defined herein or the composition or immunogenic composition or vaccine as described and/or defined herein. Usually, an immune or immunological response includes, but is not limited to, one or more of the following effects: the production or activation of antibodies, B cells, helper T cells, suppressor T cells, and/or cytotoxic T cells, directed specifically to an antigen or antigens included in the one or more ASFV as described and/or defined herein or the composition or immunogenic composition or vaccine as described and/or defined herein. Preferably, the host will display either a therapeutic or a protective immunological (memory) response such that resistance to new infection will be enhanced and/or the clinical severity of the disease reduced. Such protection will be demonstrated by either a reduction in number of symptoms, severity of symptoms, or the lack of one or more of the symptoms associated with the infection of the wild-type ASFV, a delay in the of onset of viremia, reduced viral persistence, a reduction in the overall viral load and/or a reduction of viral excretion.

In the context of the present invention, the term “a pharmaceutically acceptable or veterinary-acceptable carrier” includes any and all solvents, dispersion media, coatings, adjuvants, stabilizing agents, diluents, preservatives, antibacterial and antifungal agents, isotonic agents, adsorption delaying agents, and the like. In some preferred embodiments, and especially those that include lyophilized immunogenic compositions, stabilizing agents for use in the present invention include stabilizers for lyophilization or freeze-drying. In some embodiments, the immunogenic composition of the present invention contains an adjuvant. “Adjuvants” as used herein, can include aluminum hydroxide and aluminum phosphate, saponins e g , Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of Carbopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide, or naturally occurring or recombinant cytokines or analogs thereof or stimulants of endogenous cytokine release, among many others.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 0.01 to 50%, preferably at a concentration of about 2% to 30%, more preferably at a concentration of about 5% to 25%, still more preferably at a concentration of about 7% to 22%, and most preferably at a concentration of 10% to 20% by volume of the final product.

In the context of the present invention, the term “diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

In the context of the present invention, the term “attenuation” means reducing the virulence of a pathogen. In the present invention, an attenuated ASFV is one in which the virulence has been reduced so that it does not cause clinical signs of an African swine fever infection but is capable of inducing an immune response in the target mammal, but may also mean that the clinical signs are reduced in incidence or severity in animals infected with the attenuated ASFV in comparison with a “control group” of animals infected with non-attenuated (wild-type) ASFV and not receiving the attenuated ASFV. In this context, the term “reduce/reduced” means a reduction of at least 10%, preferably 25%, even more preferably 50%, still more preferably 60%, even more preferably 70%, still more preferably 80%, even more preferably 90% and most preferably of 100% as compared to the control group as defined above. Thus, an attenuated ASFV isolate is one that is suitable for incorporation into an immunogenic composition comprising the one or more ASFV as described and/or defined herein.

In the context of the present invention, the term “effective dose” means, but is not limited to, an amount of antigen that elicits, or is able to elicit, an immune response that yields a reduction of clinical symptoms in an animal to which the antigen is administered.

In the context of the present invention, the term “effective amount” means, in the context of a composition, an amount of an immunogenic composition capable of inducing an immune response that reduces the incidence of or lessens the severity of infection or incident of disease in an animal. Particularly, an effective amount refers to plaque forming units (pfu) per dose. Alternatively, in the context of a therapy, the term “effective amount” refers to the amount of a therapy which is sufficient to reduce or ameliorate the severity or duration of African swine fever, or one or more symptoms thereof, prevent the advancement of such disease, cause the regression of such disease, prevent the recurrence, development, onset, or progression of one or more symptoms associated with such disease, or enhance or improve the prophylaxis or treatment of another therapy or therapeutic agent.

In a preferred embodiment, the ASFV as described and/or defined herein is provided, wherein the non-functional genomic CD2 gene, if functional or complemented by a functional non-genomic CD2 gene, confers immunoprotection for such ASFV in a host, i.e. protection of such ASFV from the cellular immune response or the cellular and humoral immune response of the host against such ASFV.

In the context of the present invention, the term “complemented by a functional non-genomic CD2 gene” refers to, but is not limited to, complementation of the non-functional genomic ASFV CD2 gene by introduction of expression constructs, such as vectors and/or plasmids, encoding a functional CD2 gene, preferably a functional ASFV CD2 gene.

In a preferred embodiment, the ASFV as described and/or defined herein is provided, wherein the non-functional genomic CD2 gene comprises or preferably consists of a nucleic acid sequence according to SEQ ID NO 1: AATATTTCGCTTATTCATGTAGATAGAATTATTTAA

This sequence corresponds with the last 36 nucleotides from the EP402R ORF (GenBank entry: L16864.1), encoding for the ASFV CD2.

In a preferred embodiment, the ASFV as described and/or defined herein is provided, wherein such ASFV only comprises a non-functional genomic CD2 gene and does non comprise any further non-functional genomic genes.

In yet a preferred embodiment, the ASFV as described and/or defined herein is provided, wherein such ASFV comprises a non-functional genomic CD2 gene, preferably EP402R, and a functional genomic C-type lectin gene, preferably EP153R.

In a preferred embodiment, the ASFV as described and/or defined herein is provided, wherein such ASFV comprises a non-functional genomic CD2 gene and additionally one or more further non-functional genomic genes.

In the context of the present invention, the term “any further non-functional genomic gene” in connection with the ASFV as described and/or defined herein refers to one or more modified genes other than CD2 located in the genome of an ASFV, preferably a non-naturally occurring recombinant ASFV, wherein such modification of such ASFV genes results in no ASFV gene product at all or a biologically not functional ASFV gene product as compared to a given non-modified functional ASFV gene. Including but without being limited to that such modification can be for instance a full or partial deletion of the genomic ASFV gene and/or the modification of one or more nucleotides controlling and/or encoding the corresponding ASFV gene product and/or disruption of the respective ASFV open reading frame (ORF), for instance by inserting one or more nucleotides into that ASFV ORF, and/or any other currently known or conceivable method of inactivating or knocking-out such ASFV gene.

In a preferred embodiment, the ASFV as described and/or defined herein is provided, wherein such ASFV is a virulent and/or attenuated European or African ASFV isolate. Preferably such ASFV is a virulent isolate of ASFV selected from the group consisting of: BA71, E70, E75, E75L, Malawi Li1-20/1, OURT 88/1, OURT 88/3, Benin 97/1, Georgia 2007/1, Pretorisuskop/96/4,3, Warthog, Warmbaths, Mkuzi 1979, Tengani 62, Kenya 1950; more preferably BA71.

In a preferred embodiment, the ASFV as described and/or defined herein is provided, wherein such ASFV is ASFV isolate BA71ΔCD2, preferably BA71.ΔCD2 [deposited on 14 Mar. 2014 under identification reference “BA71.ΔFx” at the Collection Nationale de Cultures de Microorganisms (CNCM) of the Institut Pasteur under accession number CNCM I-4843 by Maria Luisa Salas, worker of Agencia Estatal Consejo Superior de Investigaciones Científicas (CSIC) in its Centro de Biologia Molecular Severo Ochoa, addressed at Nicolás Cabrera, 1, 28049 Madrid (Spain)].

In a preferred embodiment, the one or more ASFV or the immunogenic composition or the vaccine as described and/or defined herein are provided, wherein the one or more ASFV is to be administered, directly or as part of the composition or immunogenic composition or vaccine, in a dose of from 10 to 10⁸ plaque forming units (pfu), preferably 10, 10², 10³, 10⁴, 10⁵, 10⁶, 10⁷ or 10⁸ pfu, more preferably 10³ pfu. Preferably, the one or more ASFV is to be administered, directly or as part of the composition or immunogenic composition or vaccine, in a single dose or in several doses.

In the context of the present invention “pfu” is defined as “plaque forming units”, a standard value for the quantification of lytic viruses consisting of quantifying the lysis plaques provoked by the virus while infecting cell monolayers growing in semi-solid media. Under these conditions, each virus plaque is originated from one only parental virus particle.

In a preferred embodiment, the one or more ASFV or the composition or immunogenic composition or the vaccine as described and/or defined herein are provided, wherein the one or more ASFV is to be administered, directly or as part of the composition or immunogenic composition or vaccine, before, simultaneously or after the single or multiple administration of an additional immunogenic composition or vaccine, preferably before, simultaneously or after the administration of a DNA vaccine, more preferably of an ASFV-DNA vaccine. Preferably, the one or more ASFV is to be administered, directly or as part of the composition or immunogenic composition or vaccine, after the single or multiple administration of an ASFV-DNA vaccine, preferably after twice administration of an ASFV-DNA vaccine.

### Adjuvants

In order to further increase the immunogenicity of the immunogenic compositions and/or vaccines as described and/or defined herein, and which contain the one or more ASFV as described and/or defined herein, may also comprise one or more adjuvants.

The adjuvant may be purified by any of the techniques described previously or known in the art. The preferred purification technique is silica gel chromatography, in particular the “flash” (rapid) chromatographic technique. However, other chromatographic methods, including HPLC, may be used for purification of the adjuvant. Crystallization may also be used to purify the adjuvant. In some cases, no purification is required as a product of analytical purity is obtained directly from the synthesis.

The immunogenic compositions and/or vaccines as described and/or defined herein are prepared by physically mixing the adjuvant with the ASFV as described and/or defined herein under appropriate sterile conditions in accordance with known techniques to produce the adjuvanted composition.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 0.01% to 75%, preferably at a concentration of about 2% to 30%, more preferably at a concentration of about 5% to 25%, still more preferably at a concentration of about 7% to 22%, and most preferably at a concentration of 10% to 20% by volume of the final product.

### Physiologically Acceptable Vehicles

The immunogenic compositions and/or vaccines as described and/or defined herein may be formulated using techniques similar to those used for other pharmaceutical compositions. Thus, the adjuvant and the one or more ASFV as described and/or defined herein may be stored in lyophilized form and reconstituted in a physiologically acceptable vehicle to form a suspension prior to administration. Alternatively, the adjuvant and the one or more ASFV as described and/or defined herein may be stored in the vehicle. Preferred vehicles are sterile solutions, in particular, sterile buffer solutions, such as phosphate buffered saline. Any method of combining the adjuvant and the one or more ASFV as described and/or defined herein in the vehicle such that improved immunological effectiveness of the immunogenic composition is appropriate.

The volume of a single dose of the compositions and/or immunogenic compositions and/or vaccines as described and/or defined herein may vary but will be generally within the ranges commonly employed in conventional vaccines. The volume of a single dose is preferably between about 0.1 ml and about 3 ml, preferably between about 0.2 ml and about 1.5 ml, more preferably between about 0.2 ml and about 0.5 ml at the concentrations of the one or more ASFV as described and/or defined herein and adjuvant noted above.

The compositions and/or immunogenic compositions and/or vaccines as described and/or defined may be administered by any convenient means.

### Formulation

The formulations of the invention comprise an effective immunizing amount of the compositions and/or immunogenic compositions and/or vaccines as described and/or defined herein and a physiologically acceptable vehicle. Vaccines comprise an effective immunizing amount of the immunogenic compositions as described and/or defined herein and a physiologically acceptable vehicle. The formulation should suit the mode of administration.

The compositions and/or immunogenic compositions and/or vaccines as described and/or defined herein, if desired, can also contain minor amounts of wetting or emulsifying agents, or pH buffering agents. The compositions and/or immunogenic compositions and/or vaccines as described and/or defined herein can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

### Effective Dose

The compositions and/or immunogenic compositions and/or vaccines and/or one or more ASFV as described and/or defined herein can be administered to a mammal at therapeutically effective doses to treat African swine fever. The dosage will depend upon the host receiving the vaccine as well as factors such as the size, weight, and age of the host.

The precise amount of compositions and/or immunogenic compositions and/or vaccines and/or one or more ASFV as described and/or defined herein to be employed in a formulation will depend on the route of administration and the nature of the subject (e.g. species, age, size, stage/level of disease), and should be decided according to the judgment of the practitioner and each mammal's circumstances according to standard clinical techniques. An effective immunizing amount is that amount sufficient to treat and/or prevent an African swine fever infection in a mammal. Effective doses may also be extrapolated from dose-response curves derived from animal model test systems and can vary from 0.001 mg/kg to 100 mg/kg.

Toxicity and therapeutic efficacy of the compositions and/or immunogenic compositions and/or vaccines and/or one or more ASFV as described and/or defined herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions and/or immunogenic compositions and/or vaccines and/or one or more ASFV as described and/or defined herein which exhibit large therapeutic indices are preferred. While compositions and/or immunogenic compositions and/or vaccines and/or one or more ASFV as described and/or defined herein that exhibit toxic side effects can be used, care should be taken to design a delivery system that targets such compositions and/or immunogenic compositions and/or vaccines and/or one or more ASFV as described and/or defined herein to the site of affected tissue in order to minimize potential damage to uninfected cells and, thereby, reduce side effects.

The data obtained from the cell culture assays and animal studies can be used in formulating a range of dosage for use in mammals. The dosage of such compounds lies preferably within a range of circulating concentrations that include the ED50 with little or no toxicity. The dosage can vary within this range depending upon the dosage form employed and the route of administration utilized. For any compositions and/or immunogenic compositions and/or vaccines and/or one or more ASFV as described and/or defined herein used in the methods of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in mammals. Levels in plasma can be measured, for example, by high performance liquid chromatography.

Immunogenicity of a composition can be determined by monitoring the immune response of test subjects following immunization with the composition by use of any immunoassay known in the art. Generation of a humoral (antibody) response and/or cell-mediated immunity, may be taken as an indication of an immune response. The immune response of the test subjects can be analyzed by various approaches such as: the reactivity of the resultant immune serum to the immunogenic composition, as assayed by known techniques, e.g., enzyme linked immunosorbent assay (ELISA), immunoblots, immunoprecipitations, ELISPOTs, lymphoproliferation assays, etc.; or, by protection of immunized hosts from infection by the pathogen and/or attenuation of symptoms due to infection by the pathogen in immunized hosts as determined by any method known in the art, for assaying the levels of an infectious disease agent, e.g., the viral ASFV levels (for example, by culturing of a sample from the subject), or other technique known in the art. The levels of the infectious disease agent may also be determined by measuring the levels of the antigen against which the immunoglobulin was directed. A decrease in the levels of the infectious disease agent or an amelioration of the symptoms of the infectious disease indicates that the composition is effective.

The therapeutics of the invention can be tested in vitro for the desired therapeutic or prophylactic activity, prior to in vivo use in animals. For example, in vitro assays that can be used to determine whether administration of a specific therapeutic is indicated include in vitro cell culture assays in which appropriate cells from a cell line or cells cultured from a subject having a particular disease or disorder are exposed to or otherwise administered a therapeutic, and the effect of the therapeutic on the cells is observed.

Alternatively, the therapeutic may be assayed by contacting the therapeutic to cells (either cultured from a subject or from a cultured cell line) that are susceptible to infection by the infectious disease agent but that are not infected with the infectious disease agent, exposing the cells to the infectious disease agent, and then determining whether the infection rate of cells contacted with the therapeutic was lower than the infection rate of cells not contacted with the therapeutic. Infection of cells with an infectious disease agent may be assayed by any method known in the art.

After vaccination of a mammal to an ASFV using the methods and compositions of the present invention, any binding assay known in the art can be used to assess the binding between the resulting antibody and the particular ASFV. These assays may also be performed to select antibodies that exhibit a higher affinity or specificity for the particular antigen.

### Administration

Preferred routes of administration include but are not limited to intranasal, oral, intradermal, and intramuscular. Administration in drinking water, most preferably in a single dose, is desirable. The skilled artisan will recognize that the compositions and/or immunogenic compositions and/or vaccines and/or one or more ASFV as described and/or defined herein may also be administered in one, two or more doses, as well as, by other routes of administration. For example, such other routes include subcutaneously, intracutaneously, intravenously, intravascularly, intraarterially, intraperitoneally, intrathecally, intratracheally, intracutaneously, intracardially, intralobally, intramedullarly, intrapulmonarily, and intravaginally. Depending on the desired duration and effectiveness of the treatment, the compositions according to the invention may be administered once or several times, also intermittently, for instance on a daily basis for several days, weeks or months and in different dosages.

### BRIEF DESCRIPTION OF THE DRAWINGS

FIG. 1: Upper panel: The recombination plasmid contains the repressor+selection cassette that consists of the Lac I repressor gene under the control of the viral early/late promoter pU104, and the β-glucuronidase (β-gus) gene under the control of the late p72 promoter. The plasmid also contains the recombination regions constituted by genes EP152R and EP153R at the left, and gene EP364R and the last 36 bp of gene EP402R at the right. Lower panel: The resulting ASFV recombinant isolate BA71.ΔCD2, obtained by homologous recombination of BA71 with the recombination plasmid. EP402R is the ASFV gene encoding CD2.

FIG. 2: Summary of the information for each pig including identification number, immunization group, and the viruses used for challenge.

FIG. 3: Summary of the experimental design of the ASFV challenges.

FIG. 4: BA71ΔCD2 protects against homologous and heterologous ASFV challenges.

FIG. 5: Surviving pigs control viremia: LAV 8 and 29 did not show viremia after BA71 and E75 challenge, respectively (arrows), while LAV 11 showed shorter and lower ASFV titres in blood (punctuated ellipses) than pigs dying from infection; GEC/ml: genomic equivalent copies per millilitre of serum (measured by RT PCR)

FIG. 6: Surviving pigs control fever: LAV 8 and LAV29 did not show any fever after BA71 and E75 challenge, respectively, while LAV11 showed shorter and lower peaks of fever (punctuated ellipses) than pigs dying from ASFV infection.

FIG. 7: Surviving pigs showed detectable anti-ASFV antibodies at the time of homologous (continuous red line) and heterologous (dotted red-line) challenges.

FIG. 8: Correlation between protection and ASFV specific T-cell levels present at the time of challenge.

FIG. 9: BA71ΔCD2 induces polyclonal CD8 T-cells that recognize both homologous and heterologous ASFV isolates.

FIG. 10: BA71ΔCD2 protects against homologous (BA71) and heterologous (E75) lethal challenge.

FIG. 11: BA71ΔCD2 protects in a dose dependent manner

FIG. 12: BA71ΔCD2 protects against ASFV isolate Georgia 2007.

FIG. 13: BA71ΔCD2 protects against heterologous ASFV isolate Georgia 2007 lethal challenge. Surviving pigs control viremia: pig numbers (series) 4, 8 and 9 did not show viremia after Georgia 2007 challenge, while pigs numbers 1, 2, 3, 6 and 7, showed shorter and lower ASFV titres in blood (punctuated ellipses) than control pigs (green lines), dying from infection; GEC/ml: genomic equivalent copies per millilitre of serum (measured by RT PCR)

FIG. 14: Rectal temperature (only clinical sign in vaccinated pigs) coincides with viremia. Surviving pigs control fever: pig numbers (series) 4, 8, 9 and 10 did not show any fever after Georgia 2007 challenge, while pigs 1, 2, 3, 6 and 7 showed shorter and lower peaks of fever than control pigs (punctuated ellipses) dying from the infection.

### EXAMPLES

The following examples serve to further illustrate the present invention; but the same should not be construed as a limitation of the scope of the invention disclosed herein.

### Example 1

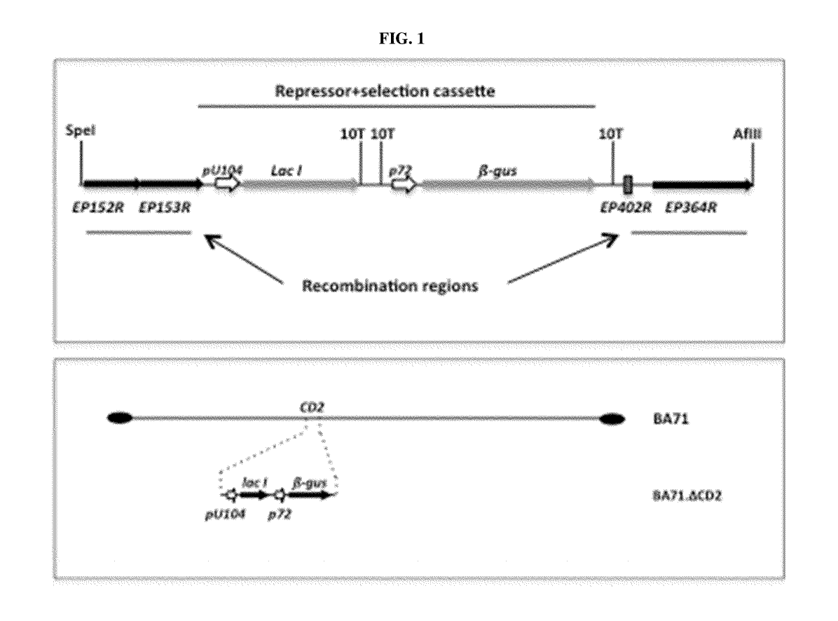

For the construction of the recombinant by homologous recombination, the recombination plasmid shown in the upper panel of FIG. 1 was used. This plasmid contains the repressor+selection cassette consisting of the Lac I repressor gene under the control of the ASFV early/late promoter pUl 04 and the marker β-glucuronidase gene under the control of the late p72 promoter. The plasmid also contains the recombination regions that consist of genes EP152R and EP153R genes at the left, and, at the right, the EP364R gene and a 36 base pair region at the end of gene EP402R coding for the ASFV CD2.

The BA71.ΔCD2 (FIG. 1, lower panel) was obtained by homologous recombination of the recombination plasmid with BA71 in COS-7 cells. The recombinant virus was purified by successive plaque formation in COS-7 cells, selecting blue plaques stained with X-Gluc, the substrate of β-glucuronidase, until only blue plaques are detected. The ASFV was amplified by growth in COS-7 cells.

To produce the virus recombinant BA71.ΔCD2 in large amounts for in vivo inoculation studies, preconfluent monolayers of COS-7 cells (25 P150 plates) were infected with BA71.ΔCD2 at a multiplicity of infection of 0.1 plaque forming units (pfu) per cell. After a severe cytopathic effect was observed, the culture medium containing the extracellular virus was collected and centrifuged at low speed to remove cellular debris and then at high speed to sediment the virus. The sediment was resuspended in PBS and used for the protection experiments after titration by formation of lysis plaques. In earlier experiments, after resuspending in PBS, the virus was purified by centrifugation on a 25% saccharose cushion in PBS. The sediment obtained was resuspended in PBS and titrated as before. The virus concentration is expressed as plaque forming units (pfu) per ml.

### Example 2

Once obtained, the BA71.ΔCD2, purified or not, was used for vaccine purposes attending the following experimental design:
Twenty-four (24) Landrace x Pietrain commercial pigs (four week old males) were hosted in two boxes (12 pigs per box): BOX A and BOX B, within BSL3-facilities.
Each box was divided into 2 immunization groups (6 pigs in each group):
   Control Group (C): Intramuscularly immunized with PBS
   Live Attenuated Virus (LAV): Intramuscularly immunized with 10³ pfu of the ASFV CD2-deletion mutant BA71ΔCD2
All pigs in box A were challenged intramuscularly with a lethal dose of 10³ Haemadsorbing units (HAU₅₀) of the virulent BA71 ASFV isolate (20LD₅₀ homologous challenge)
All pigs in box B were challenged intramuscularly with a lethal dose of 10⁴ HAU₅₀ of the virulent E75 ASFV isolate (20LD₅₀ heterologous challenge)

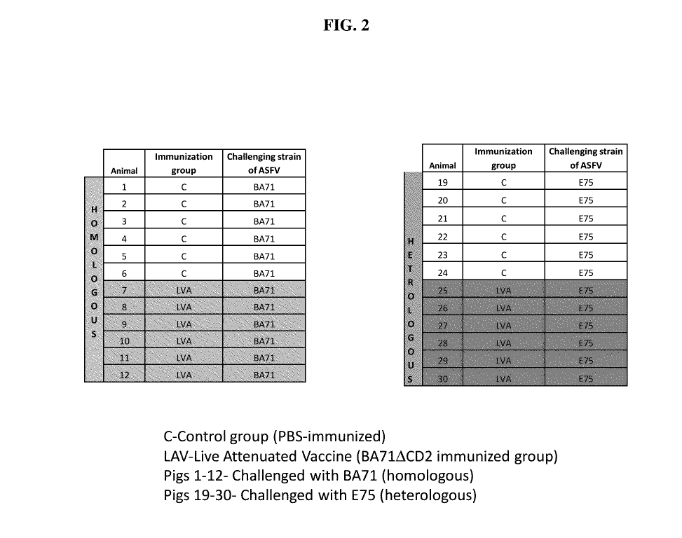

FIG. 2 summarizes the information for each pig including identification number, immunization group, and the viruses used for challenge.

The absence of the ASFV CD2 gene made it impossible to titrate BA71.ΔCD2 according to Haemadsorbing units (HAU₅₀), whereas its adaptation to COS cells allowed the quantification of the viral stocks in plaque forming units (pfu) using an standardized plaque assay. Conversely, E75 only grows in primary porcine macrophages, not forming plaques; therefore E75 was titrated according to Haemadsorbing units (HAU₅₀) [1 pfu is equivalent to 1 HAU₅₀ and to 1 Genome equivalent copy (GEC)].

### ASFV Lethal Challenge

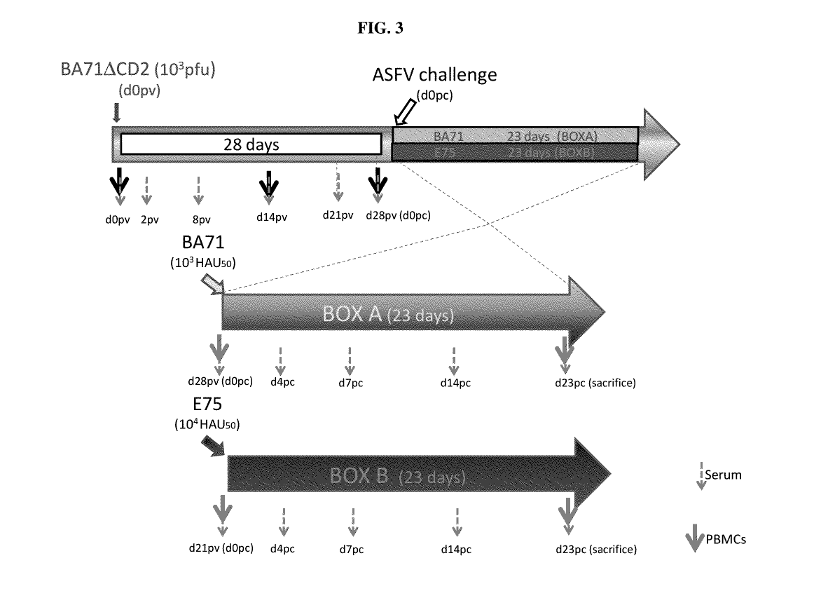

28 days after BA71ΔCD2 inoculation, pigs were challenged intramuscularly with a lethal dose of ASFV*:
Pigs in BOX A were challenged with 10³ HAU₅₀ of virulent BA71, the parental isolate of BA71ΔCD2 (20LD₅₀ of the homologous virus)
Pigs in BOX B were challenged with 10⁴ HAU₅₀ of virulent E75 (20 LD₅₀ of the heterologous virus).
   *Please, note that to use the same lethal dose (20 LD₅₀) for ASFV challenges, a different viral dose was needed: 10³ HAU₅₀ for the BA71 and 10⁴ HAU₅₀ (10 times more virus) for the E75. Viral and death kinetics are similar when using these viruses and challenge doses. Remember also that these natural virus isolates have to be grown in primary macrophages, therefore being quantified by haemadsorption and/or RT-PCR.
   Serum and/or total blood samples were collected before BA71ΔCD2 inoculation and lethal challenge and at different time points after BA71ΔCD2 vaccination [2, 8, 14, 21 and 28 days post-vaccination (pv)] and after lethal challenge [4, 7, 14 and 23 days post-challenge (pc)]. Rectal temperature was daily recorded.
   The experimental design is schematically summarized in the FIG. 3.

### Analytical Determinations

Viral Detection: Viremia was quantified by using a tailor-made real time PCR specific for the ASFV serine protein kinase gene. Briefly, the viral nucleic acid was obtained from sera using the NucleoSpin Blood Kit (Macherey-Nagel) and quantitative the SybrGreen qPCR (Applied Bioscience) was performed using the forward 5′-CCTTTCCACCTTTGCTGTAGGA and reverse 5′-GTCCAGGCCGGAACAACA primers, to amplify a 85 bp from the highly conserved ASFV serine protein kinase gene (R298L). All samples were assayed in triplicates, including the negative and positive controls. The standard curve was performed using the purified p-R298, containing the full length R298L ORF (from 10² to 10¹⁰ GEC/ml). The limit of detection of the technique (100% confidence) is 1,000 genome equivalent copies per millilitre of serum (GEC/ml). One GEC was equivalent to one Haemagglutinin Unit (HAU), the classical way for detecting live infectious virus in a complex sample. All samples negative for RT-PCR were also negative in the haemagglutinin assay (data not shown).
Antibody Detection: All sera were subjected to the OIE-approved ELISA for ASF diagnosis. All serum samples were tested at a 1:100 dilution.
T-Cell Detection: PBMCs isolated from pigs at different days after vaccination and/or infection were subjected to:
   An IFN-gamma ELISPOT assay to detect the presence of specific T-cells in blood at different days post immunization and post infection. PBMCs were overnight (O/N) stimulated in vitro with live ASFV, either using 10⁵ HAU₅₀/million PBMCs of the virulent BA71 isolate or the same amount of the E75 isolate (similar results were obtained irrespective of the virus used).
   For some samples, a Carboxyfluorescein Diacetate Succinimidyl Ester (CFSE) proliferation assay was performed to evaluate the presence of cross-reactive CD8⁺ T-cells in blood, again using BA71 or E75 as specific stimuli for 5 days.

By using this experimental approach, the following results were obtained:

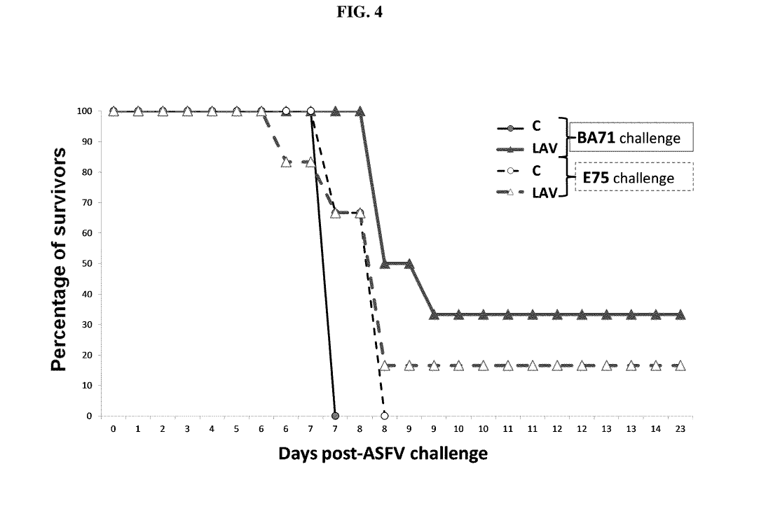

BA71ΔCD2 Protects Pigs From Homologous and Heterologous Lethal Challenges: As expected, all control pigs (C) died before day 7 post-challenge (pc) with a lethal dose of 10³ HAU50 of the virulent BA71 isolate (FIG. 4, black continuous line). By that time (day 7 pc), 3 out of the 6 pigs receiving 10³ HAU₅₀ of BA71ΔCD2 remained alive and more importantly, 2 of them survived (FIG. 4, red continuous line).

In contrast with lack of cross-protection observed by immunization with the classically attenuated E75CV1 isolate (data not shown), BA71ΔCD2 was capable of protecting against the E75 heterologous lethal challenge. In this occasion, 1 out of 6 pigs vaccinated with 10³ pfu of BA71ΔCD2 survived the lethal challenge with E75 (discontinuous red line). As expected for E75 challenge, all control pigs (C) died between days 7 and 8 pc.

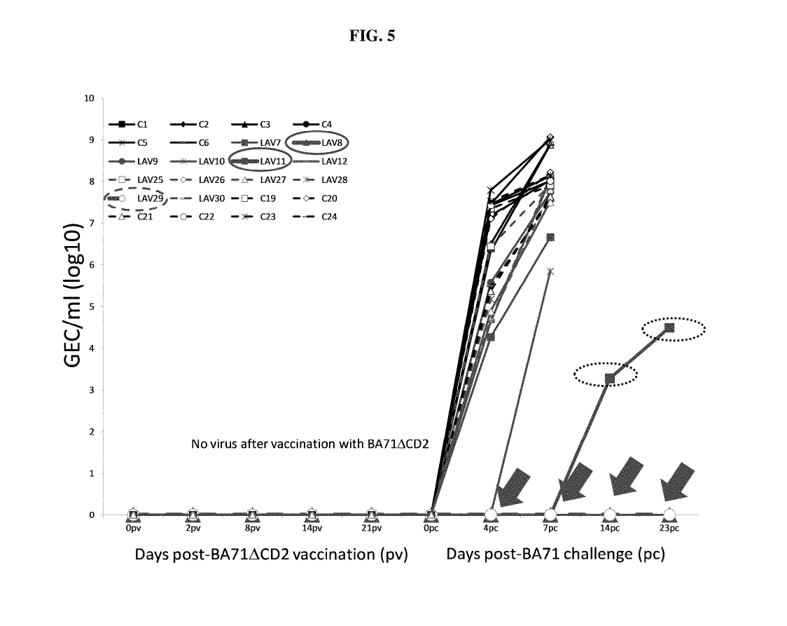

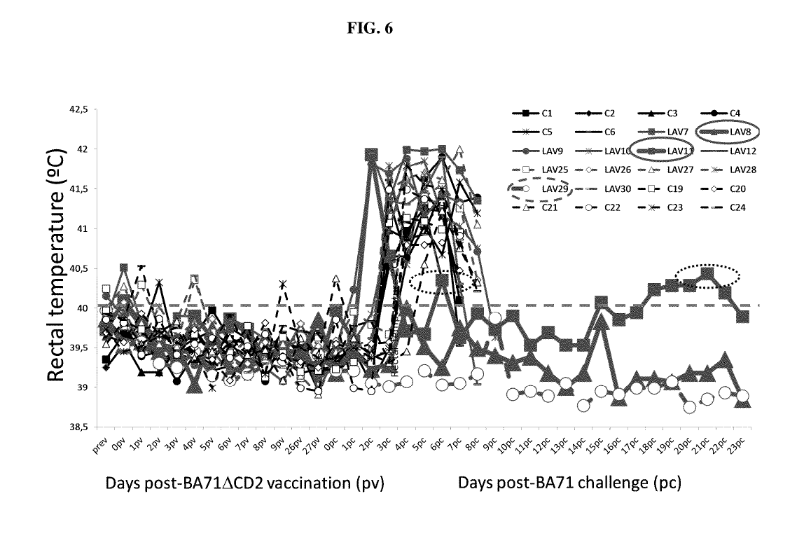

BA71ΔCD2 is Safe in Pigs and Can Reduce or Prevent Viremia After Homologous or Heterologous Lethal Challenges: Pigs intramuscularly inoculated with 10³ pfu of BA71ΔCD2 did neither show any detectable viremia (FIG. 5), neither any other clinical signs compatible with ASF, including fever (FIG. 6). Control pigs (black lines) became sick very soon after BA71 or E75 challenge, showing very high levels of viremia by day 4 post challenge and reaching their maximum at the time of death (day 7 pc). In contrast, most of the pigs vaccinated with BA71ΔCD2 showed lower levels of viremia than control pigs, differences that became more evident by day 4 post infection (FIG. 5, red lines). Interestingly enough, two of the three surviving pigs (LAV8 and LAV29), did not show detectable viremia at any time post infection, while the other survivor (LAV11) showed a lower and shorter peak of viremia than control pigs (FIG. 5, black doted elypse). For the first time using live attenuated vaccines sterilizing immunity against the heterologous challenge was observed for pig LAV29.

Viremia results (considering 0 values below 10³ GEC/ml the limit of the applied detection methods) matched with rectal temperature records. Thus, while pigs dying of ASF suffered from high fever episodes very early after challenge until the time of death, surviving pigs showed if any, shorter and milder fever peaks (FIG. 6, ellipses in pig LAV11) that coincided with reduced or not-detectable viremia (FIG. 5, red lines). LAV8 and LAV 29 did not show any fever or other clinical signs typical for ASF. This, together with the fact that no virus was detectable from nasal swabs (data not shown) by either real-time PCR or by virus isolation, demonstrated their sterilizing protection (assumed considering that the limit of our detection method is 10³ GEC/ml).

BA71ΔCD2 Induces ASFV-Specific Antibodies: Once the capability of BA71ΔCD2 of conferring full protection against both homologous and heterologous lethal challenges had been demonstrated, the key question to be answered was why not all animals became equally protected. Aiming at answering this question, the specific antibody responses induced in each of the immunized animals throughout the experiment was monitored by using a specific ELISA (www.oie.int).

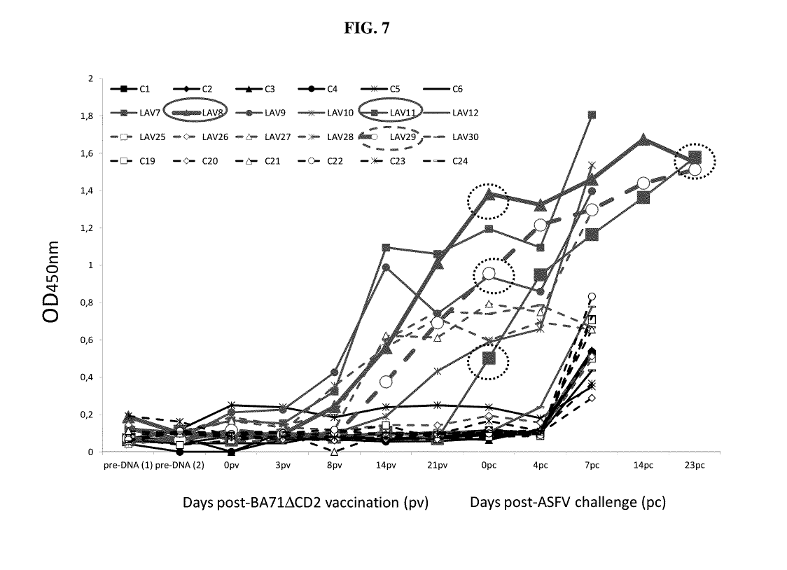

As expected, control pigs did not show any specific reaction until day 7 pc at necropsy time, when they showed low albeit detectable antibodies against ASFV (FIG. 7), independently of the viruses used for challenge: BA71 (continuous black line) or E75 (dotted black line). In contrast, a large proportion of BA71ΔCD2-immunized pigs (75% or 9 out of 12 pigs) developed specific antibody responses detectable as early as at 8 days post vaccination (pv) and reaching their maximum titres at the time of challenge (d0pc or 28 dpv). These results demonstrate that BA71ΔCD2 is capable of inducing ASFV-specific antibodies even in the absence of detectable viremia (FIG. 5). Therefore, the presence of specific antibodies at the time of challenge is a good indicator of successful immunization. However, a clear correlation between the level of antibodies present at the time of ASFV challenge (d0pc) and protection does not seem to exist. As illustrative examples, LAV7 did not resist the homologous BA71-challenge in spite of showing similar levels of antibodies in its blood at the time of challenge than LAV8, the pigs that were fully protected from homologous challenge. Conversely, LAV11 survived the homologous challenge in spite of having had relatively low antibody levels present in circulation at the time of challenge, being detectable in the latter case for the first time at the same day of the challenge (d0pc) (FIG. 7).

Independently of the above mentioned results, pigs fully protected by BA71ΔCD2, showing neither viremia nor any ASF-specific clinical signs through the infection, showed high levels of specific antibodies at the time of ASFV-challenge and were boosted immediately after ASFV infection (LAV8 and LAV29).

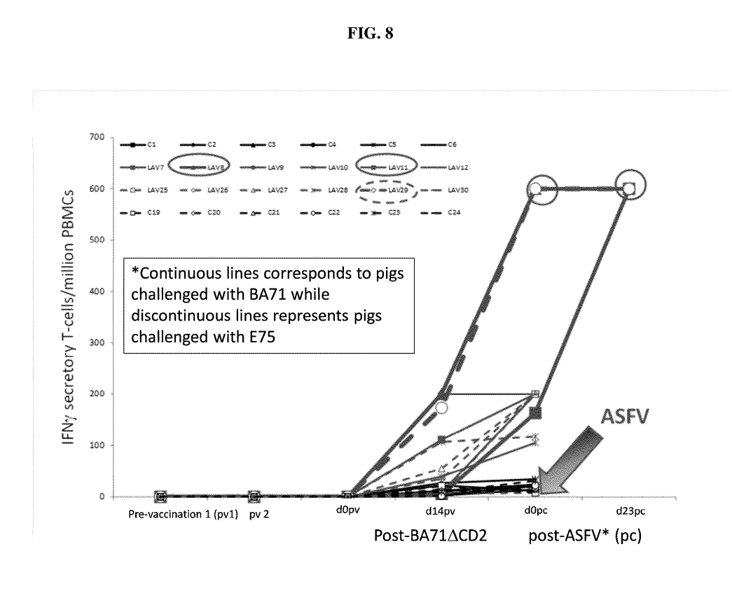

Surviving Pigs had High Numbers of Circulating ASFV-Specific T-Cells at the Time of ASFV Lethal Challenges: Aiming at establishing a correlation between protection and the induction of specific T-cell responses, an IFN-gamma ELISPOT assay was used to follow the presence of ASFV-specific T-cells in the blood of immunized pigs before and after homologous or heterologous viral challenge (FIG. 8). As expected, control pigs died before being able to show any detectable T-cell responses. In clear contrast, most BA71ΔCD2-immunized pigs developed specific T-cell responses detectable from day 14 pv on. The two pigs showing >600 specific T-cells per million of PBMCs were fully protected (LAV8 and LAV29), clearly demonstrating the relevance of this arm of the immune response in protection from ASF. Intriguingly enough, not all pigs showing moderately high specific responses at the time of challenge remained protected. Thus, while LAV11 survived showing 200 specific T-cells per million of PBMCs many others did not in spite of showing similar specific T-cells in their blood.

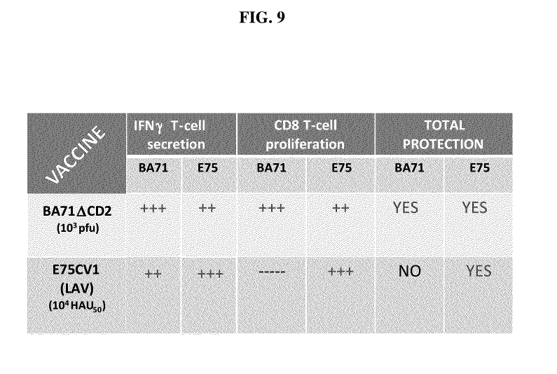

T-Cell Responses Induced by BA71ΔCD2 are Polyclonal and Cross-Reacting Against Other ASFV Isolates: Previous results (data not shown) demonstrated that infection with classically attenuated ASFV isolates (such as E75CV1) induced a very narrow T-cell repertoire against only few dominant epitopes. In fact, the ability to confer protection against the homologous but not heterologous ASFV isolates in vivo correlated with the limitation of their CD8⁺ T-cells to exclusively recognize and in vitro proliferate in response to the homologous virus. Aiming at correlating these in vitro parameters with the protection afforded by BA71ΔCD2, the following experiment was performed. PBMCs isolated from each animal before ASFV challenge, were labelled with CFSE and in vitro stimulated for five days with either BA71 or E75. Interestingly, the CD8⁺ T-cells induced by BA71ΔCD2 were capable to in vitro proliferate in response to both the homologous BA71 and the heterologous E75 virus (FIG. 9), thus correlating with the in vivo protection afforded by the vaccine against these two viruses. In contrast, CD8⁺ T-cells induced by classically attenuated E75CV1 only recognized the homologous virus (data from previous experiments; FIG. 9).

### Example 3

In order to demonstrate a dose-dependent effect of BA71ΔCD2-induced protection, pigs were immunized with either 3.3×10⁴ pfu or 10⁶ pfu of BA71ΔCD2 (in Example 2, pigs received 10³ pfu of the ASFV CD2-deletion mutant BA71ΔCD2).

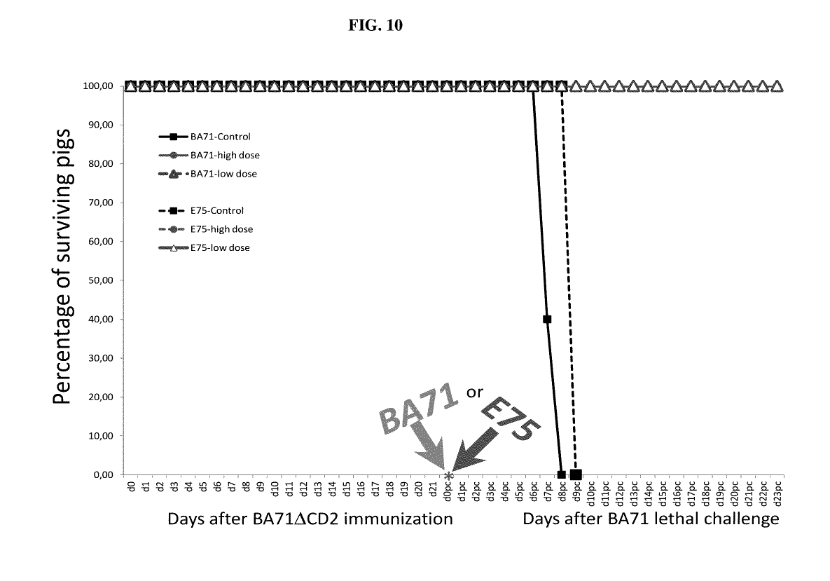

As expected, all control pigs, either challenged either with 10⁴ HAU₅₀ (GEC) of E75 (5 out of 5) or with 10³ HAU₅₀ (GEC) of BA71 (5 out of 5) died before day 9 post-challenge (pc). In clear contrast, 100% of the pigs vaccinated with BA71ΔCD2 (24 out of 24) survived the lethal challenge, independently of the vaccine dose used or the challenged performed (homologous BA71 or heterologous E75; FIG. 10). This is the first demonstration of total cross-protection for these two ASFV isolates. Protection correlated with the induction of specific antibody and T-cell responses, correlating with the protection afforded.

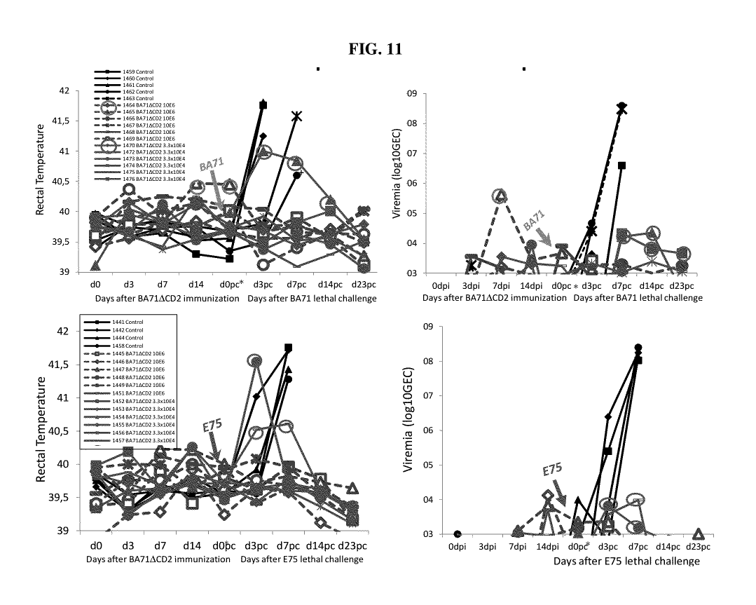

Immunization with either 3.3×10⁴ pfu or 10⁶ pfu of BA71ΔCD2 was safe for the animals with only one out of 12 pigs showing low viremia by day 7 post-vaccination (pi) and a little bit of temperature (FIG. 11, upper panel; pig number 1465, encircled). The rest of the pigs receiving BA71ΔCD2 (23 out of 24) did not show any significant viremia or clinical signs compatible with ASF, accounting for the safety of the product. Additionally, none of the control (naïve) animals kept in the same rooms resulted infected thus showing that viral secretion and transmission, if any, can be neglected.

Conversely, vaccination with 10⁶ pfu of BA71ΔCD2, conferred total protection against BA71 lethal challenge with no animals showing ASFV clinical signs or viremia at any time post infection with either the homologous BA71 challenge or the heterologous E75 challenge (red lines in upper and lower panels from FIG. 11, respectively). Albeit also very solid, the protection afforded by the lower vaccine dose (pigs received 30 times less BA71ΔCD2) was not completely sterilizing since 4 out of the 12 immunized pigs (2 per challenge group) showed some fever corresponding with very low viremia picks (4-5 logs lower of virus than control pigs) and close to the detection limit of the applied RT-PCR technique (circles in FIG. 11).

### Example 4

Despite E75 and BA71 are heterologous they belong to the same genotype and both are phylogeneticly very distant from currently circulating ASFV isolates in the Caucasus. Therefore, these experiments were designed to demonstrate the protective capability against the Georgia 2007 ASFV isolate. Two in vivo experiments were designed. The first one aiming at in vivo titration of the Georgia 2007 ASFV isolate and the second one designed to test the protective potential of BA71ΔCD2 against Georgia 2007 lethal challenge.

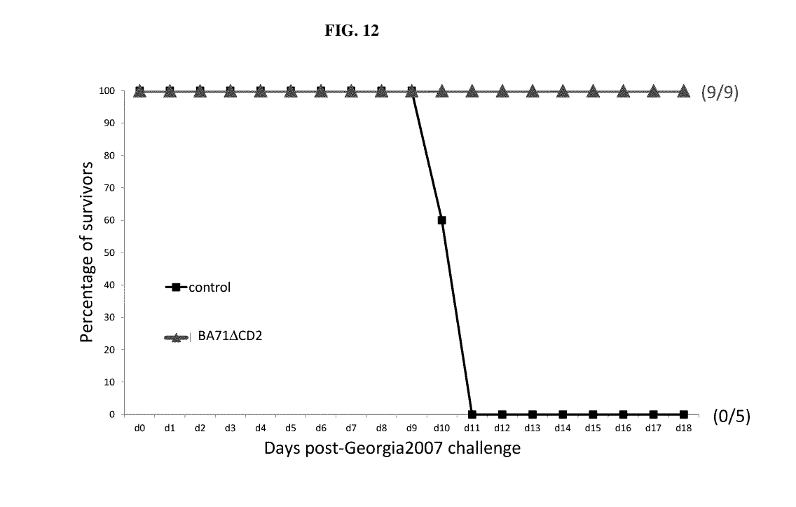

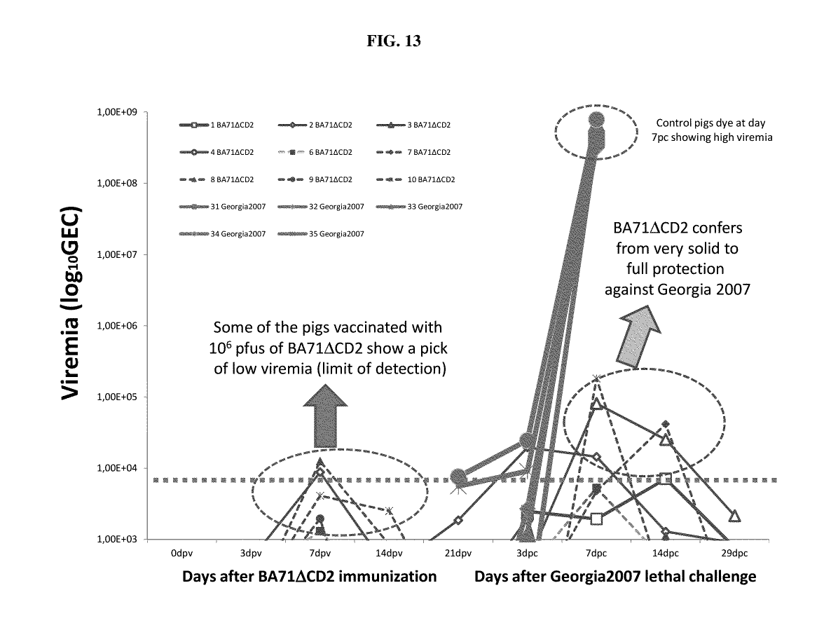

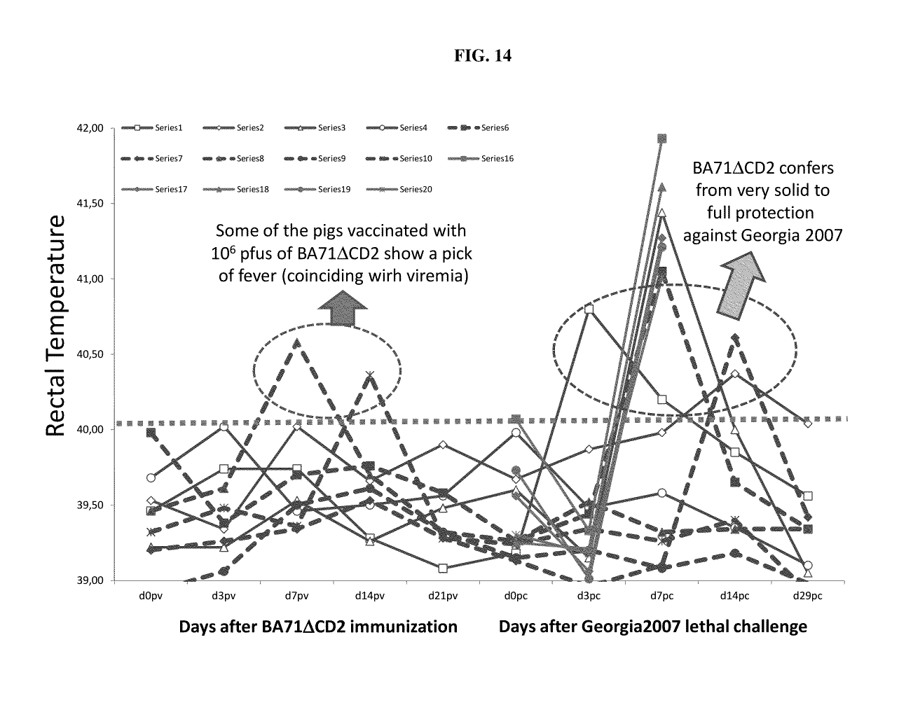

In FIG. 12, just as it occurred for BA71 and E75, it is shown that BA71ΔCD2 conferred 100% protection against Georgia 2007 lethal challenge (dose of 10³ GEC). Thus, while 5 out of 5 control pigs died before day 11 post-challenge, all pigs vaccinated with 10⁶ pfu of BA71ΔCD2 survived the same lethal dose. The main conclusions of this set of experiments are as follows:
i. The Georgia 2007 virus stock available kills all pigs in less than ten days, even at the lower dose tested 2.23×10³ GEC (genome equivalent copies as verified by RT-PCR) and showing typical acute signs of ASFV, including high fever and high viremia titres [up to 10⁹ GEC by day 7 post infection (pi)]
ii. 100% of the pigs immunized with 10⁶ pfu of BA71ΔCD2 (9 out of 9) survived the lethal Georgia 2007 challenge (intramuscular immunization with 10³ GEC), while, as expected, all control pigs (PBS inoculated) died before day 11 post-challenge.
iii. The protection afforded was comparable to that obtained against the homologous challenge since 5 of the immunized pigs showed no virus in their blood at any time tested while the other four showed very limited peak of viremia; 4 to 5 10-fold logarithms lower than control pigs (FIG. 13).
iv. Rectal temperature records confirm the results obtained with only 4 animals showing a peak of temperature corresponding with viremia, while the rest remained normal (FIG. 14). Interestingly none of the animals showed any other clinical sign compatible with ASF while all control pigs developed acute clinical signs of the disease dying before day 11 pc. Fever was the only recorded sign.

These results clearly demonstrate the protective potential of the live attenuated ASFV isolate BA71ΔCD2 against the ASFV reintroduced in Europe in year 2007 and proves that cross-protection is possible.

### REFERENCES

1. Abrams C C and Dixon L K, Virology 2012, 433 (1): 142-148
2. Andres G et al., J. Virol. 2002, 76: 2654-2666
3. Argilaguet J M, et al., PLoS One 2012, 7: e40942
4. Boinas F S et al., J General Virol 2004, 85: 2177-2187
5. Boinas F S et al., PLoS One 2011; 6 (5): e20383
6. Borca M V et al., J. Virol. 1998, 72: 2881-2889.
7. Chapman D A G et al., J General Virol 2008, 89: 397-408
8. ES 2 401 276A1
9. Escribano J M et al., Virus Research 2012, 173 (1): 101-109
10. Gomez-Puertas P et al., Virology 1998, 243: 461-471
11. Kay-Jackson P C, J General Virol 2004, 85: 119-130
12. King K et al., Vaccine 2011, 29 (28): 4593-4600
13. Lewis T et al., J Virol. 2000, 74: 1275-1285
14. Mebus C A, Adv Virus Res 1988, 35: 251-269
15. Moore D M et al., J Virol. 1998, 72 (12): 10310-10305
16. Neilan J G et al., J Virol 2002, 76: 3095-3104
17. Onisk D V et al., Virology 1994, 198: 350-354
18. Oura C A et al., J Gen Virol. 2005, 86: 2445-2450
19. Phameuropa Vol. 8, No. 2, June 1996
20. Rowlands R J et al., Virology 2009, 393 (2): 319-328
21. Ruiz-Gonzalvo F et al., In: P.J. Wilkinson (Ed.), African Swine Fever, 1983 Proc. EUR 8466 EN, CEC/FAO Research Seminar, Sardinia, September 1981, pp. 206-216
22. Ruiz-Gonzalvo F et al., Am J Vet Res 1986, 47: 1249-1252
23. Ruiz-Gonzalvo, F et al., Virology 1996, 218: 285-289
24. Stone S S et al., Am J Vet Res 1967, 28: 475-481
25. Sanchez Botija C., Zooprofilassi 1963, 18: 578-607
26. U.S. Pat. No. 2,909,462
27. Wardley R C et al., Vet Immunol Immunopathol 1985, 9: 201-212
28. WO 2012/107164

All references hereby incorporated by reference.

What is claimed is:

1. A non-naturally occurring recombinant African swine fever virus (ASFV) comprising a non-functional genomic CD2 gene, with the proviso that such ASFV is not deficient in its replication, wherein said ASFV is a live attenuated ASFV or subsequently inactivated ASFV that was yielded from the live attenuated ASFV through subsequent inactivation.

2. The ASFV according to claim 1, wherein the non-functional genomic CD2 gene is EP402R or comprises a nucleic acid sequence according to SEQ ID NO 1.

3. The ASFV according to claim 1, wherein said ASFV only comprises a non-functional genomic CD2 gene and does not comprise any further non-functional genomic genes.

4. The ASFV according to claim 1, wherein said ASFV comprises a non-functional genomic CD2 gene and a functional genomic C-type lectin gene.

5. The ASFV according to claim 4, wherein said non-functional CD2 gene comprises EP402R.

6. The ASFV according to claim 4, wherein said functional genomic C-type lectin gene comprises EP153R.

7. The ASFV according to claim 1, wherein said ASFV is a virulent European or virulent African ASFV isolate.

8. The ASFV according to claim 7, wherein said ASFV is a virulent isolate of ASFV selected from the group consisting of: BA71, E70, E75, E75L, Malawi Li1-20/1, OURT 88/1, OURT 88/3, Benin 97/1, Georgia 2007/1, Pretorisuskop/96/4,3, Warthog, Warmbaths, Mkuzi 1979, Tengani 62, Kenya 1950; more preferably BA71.

9. The ASFV according to claim 1, wherein said ASFV is ASFV isolate BA71ΔCD2.

10. The ASFV according to claim 9, wherein said ASFV is BA71.ΔCD2 (identification reference “BA71.ΔFx”, accession number CNCM 1-4843).

11. A method for the generation of a non-functional ASFV CD2 gene in an ASFV genome, comprising:
a. introducing one or more full or partial deletions into the ASFV CD2 gene;
b. modifying one or more nucleotides controlling or encoding the corresponding ASFV CD2 gene product; and/or
c. disrupting the ASFV CD2 open reading frame (ORF);
thereby rendering the ASFV CD2 non-functional.

12. The method according to claim 11, comprising introducing a Lac I repressor together with β-glucuronidase marker gene into the ASFV CD2 locus, wherein said ASFV CD2 gene is rendered substantially non-functional in vitro and in vivo.

13. A method for the production of a non-naturally occurring recombinant ASFV, comprising a non-functional genomic CD2 gene, with the proviso that such ASFV is not deficient in its replication, according to claim 1, comprising the steps of:
a. preparing a non-naturally occurring recombinant ASFV, comprising a non-functional genomic CD2 gene, according to claim 11;
b. infecting primary porcine macrophages that do not inactivate said ASFV or a cell line susceptible to infection by ASFV that does not inactivate said ASFV, with the ASFV of step (a) in vitro; and
c. isolating the ASFV from the cells of step (b) and purifying it.

14. The method according to claim 13, wherein said cell line in step (b) are COS-7 cells.

15. The method according to claim 13, wherein step (c) comprises
i. collecting the culture medium containing the extracellular ASFV;
ii. centrifuging it first at low speed to remove cellular debris and then at high speed to sediment the virus; and
iii. resuspending it in PBS.

16. The method according to claim 15, wherein said resuspended virus is further purified by centrifugation on a 25% saccharose cushion in PBS before finally resuspending the virus in PBS.

17. The method of claim 13, further comprising:
d. titrating the ASFV of step (c), by the formation of lysis plaques.

18. The method of according to claim 17, further comprising:
e. inactivating the live attenuated ASFV obtained from steps (c) or (d), thereby yielding one or more subsequently inactivated ASFV.

19. A non-naturally occurring recombinant ASFV obtainable by the method according to claim 13.

20. An immunogenic composition comprising a therapeutically effective amount of one or more ASFV, according to claim 1.

21. The immunogenic composition according to claim 20, additionally comprising one or more pharmaceutically acceptable excipients or carriers.

22. The immunogenic composition according to claim 21, wherein said one or more pharmaceutically acceptable excipients or carriers are selected from the group consisting of: solvents, dispersion media, adjuvants, stabilizing agents, diluents, preservatives, antibacterial and antifungal agents, isotonic agents, adsorption delaying agents and combinations thereof.

23. A method of treating or preventing African swine fever in mammals, comprising administering an effective dose of said one or more ASFV according to claim 1.

24. The method according to claim 23, wherein said mammal is of the family Suidae.

25. The method according to claim 24, wherein said mammal is a pig.

26. The method according to claim 25, wherein said pig is domestic pigs (Sus scrofa domesticus), wild pigs (Sus scrofa scrofa), warthogs (Potamochoerus porcus), bushpigs (Potamochoerus larvatus), giant forest hogs (Hylochoerus meinertzhageni) or feral pigs.

27. The method according to claim 23, wherein said ASFV is administered in a dose of from 10 to 10⁸ plaque forming units (pfu).

28. The method according to claim 27, wherein said ASFV is administered in a dose of 10³ pfu.

29. The method according to claim 23, wherein ASFV is administered in a single dose or in several doses.

30. The method according to claim 29, comprising further administrating another immunogenic composition.

31. The method according to claim 30, wherein said immunogenic composition is an ASFV-DNA immunogenic composition.

32. The method of claim 30, wherein said ASFV of claim 1 is administered before, simultaneously, or after the single or multiple administration of said additional immunogenic composition.

33. The method of claim 32, wherein said ASFV of claim 1 is administered after 3 doses of ASFV-DNA vaccine.

34. A method for eliciting a protective immune response in a mammal, comprising administering to said mammal the one or more ASFV according to claim 1.

35. The method according to claim 34, wherein said mammal is of the family Suidae.

36. The method according to claim 35, wherein said mammal is a pig.

37. The method according to claim 36, wherein said pig is domestic pigs (Sus scrofa domesticus), wild pigs (Sus scrofa scrofa), warthogs (Potamochoerus porcus), bushpigs (Potamochoerus larvatus), giant forest hogs (Hylochoerus meinertzhageni) or feral pigs.
